(19) United States
(12) Patent Application Publication
(10) Pub. No.: US 2026/0048205 A1
(43) Pub. Date: Feb. 19, 2026

(54) SYSTEMS AND METHODS FOR MIXING SYRINGE VALVE ASSEMBLIES AND SYRINGE FORMULATIONS

(71) Applicant: Tolmar International Limited, Dublin 2 (IE)

(72) Inventors: Harold Casey Dean, Fort Collins, CO (US); Kevin Barker, Fort Collins, CO (US); David Dummer, Fort Collins, CO (US); Nicholas DeWeerd, Fort Collins, CO (US); Amy Van Hove, Fort Collins, CO (US); Nicole Marquardt, Fort Collins, CO (US); Erik Johnson, Fort Collins, CO (US); Travis Kauffman, Fort Collins, CO (US); Dileep R. Janagam, Fort Collins, CO (US); John Charles Middleton, Fort Collins, CO (US)

(21) Appl. No.: 19/362,940

(22) Filed: Oct. 20, 2025

Related U.S. Application Data
(63) Continuation of application No. PCT/IB2025/052819, filed on Mar. 18, 2025.
(60) Provisional application No. 63/568,654, filed on Mar. 22, 2024.
(60) Provisional application No. 63/683,050, filed on Aug. 14, 2024.
(60) Provisional application No. 63/742,886, filed on Jan. 8, 2025.

(51) Int. Cl. A61M 5/315; A61M 39/00; A61M 39/22

(52) U.S. Cl. CPC A61M 5/31596; A61M 39/223; A61M 2039/0036; A61M 2202/0007; A61M 2202/04

(57) ABSTRACT

Described are syringe-to-syringe mixing systems, corresponding formulations, and methods of use including treatment methods.

### CROSS-REFERENCE TO RELATED APPLICATIONS

This application claims priority to U.S. Provisional Application No. 63/568,654, filed Mar. 22, 2024, U.S. Provisional Application No. 63/683,050, filed Aug. 14, 2024, and U.S. Provisional Application No. 63/742,886, filed Jan. 8, 2025, each of which is incorporated into this application in its entirety.

### FIELD

The present disclosure relates generally to systems for mixing and to mixing syringe systems. More specifically, embodiments of the present disclosure relate to mixing syringes operable to store and selectively mix contents between two syringes, and which include a valve assembly to separate syringe contents at least prior to mixing operations.

### BACKGROUND

It is known to store drugs and therapeutic agents in a lyophilized or powdered form. For example, because of stability and shelf-life factors, therapeutic proteins are often formulated as powders that must be reconstituted, e.g. in a liquid or flowable medium or material, prior to injection. Similarly, it is known to store components of certain complex drug formulations (whether liquid or solid) in separate containers for reasons related to stability or reactivity of the components, where the contents of the separate containers must be mixed prior to injection. Related methods and systems have been provided to allow users and healthcare professionals to combine and formulate a drug just prior to administration. Known systems and methods include single-barrel systems such as those shown and described in U.S. Pat. No. 9,592,343 to Shetty et al., which is hereby incorporated by reference. Such systems and methods generally comprise two or more contents initially separated by a valved stopper or similar member and wherein repetitive motion of the stopper and associated plunger rod is operable to mix syringe contents and subsequently eject a solution from the device. Dual-syringe systems and methods are also known, such as those shown and described in U.S. Pat. No. 9,220,577 to Jessop et al, which is hereby incorporated by reference in its entirety. Dual-syringe systems generally comprise a first material provided in a first syringe barrel and a second material provided in a second syringe barrel. The two syringe barrels of such systems can be connected to one another by joining their distal outlets such that the two syringes are in fluid communication. Sequential activation of the plunger rods of each syringe is then operable to force contents between the two syringes and provide a mixing action.

However, various known systems and devices suffer from drawbacks including, for example, a high level of pre-administration steps that can increase the likelihood of user error, improper mixing, dosing errors, and unwanted migration or leakage of syringe contents. For example, existing devices may initially be provided to a user as two separate syringes, each housing separate contents, e.g. a liquid or flowable component and a lyophilizate or other form of therapeutic agent, in order to reduce or eliminate the risk of unwanted migration of the liquid or flowable component from a first syringe to the lyophilizate or other form of therapeutic agent of a second syringe during storage. However, the use of such a device requires additional steps, including assembly steps, before mixing and administration can occur. Conversely, single-barrel systems reduce or eliminate the need to connect or assemble components prior to mixing but provide for the possibility that a liquid or flowable component can migrate or leak into a volume containing lyophilizate or other form of therapeutic agent prior to the desired time for mixing. Single-barrel systems are also restricted by the viscosity of syringe contents and are not suitable for all applications.

### SUMMARY

Accordingly, there has been a long-felt and unmet need to provide methods and systems for mixing drugs and therapeutic agents while reducing the risks and drawbacks of methods and systems of the prior art.

It is an object of the present disclosure to provide methods and systems for mixing contents while reducing the number of required administration steps. It is also an object of the present disclosure to provide methods and systems for mixing contents which reduce the risk of adverse user errors and mistakes associated with administration of said contents. It is a further object of the present disclosure to provide methods and systems for mixing the contents of containers, chambers, or syringes while preventing or reducing the risk of unwanted migration, combination, mixing, etc. of materials. Furthermore, it is an object of the present disclosure to provide methods and systems for mixing portions of a pharmaceutical composition or formulation comprising an active pharmaceutical ingredient (API) useful in the treatment in a disease or disorder in a patient, wherein the API may be presented in a solid or liquid form prior to formulation reconstitution via mixing. It is yet a further object of the present disclosure to provide methods and systems for mixing materials of a pharmaceutical formulation comprising lyophilized, suspended, or dissolved leuprolide or a pharmaceutically acceptable salt thereof (e.g. leuprolide acetate) and a biodegradable polymer-solvent system useful in the treatment of a disease or a disorder, including a cancer, including but not limited to prostate cancer or breast cancer. In some embodiments, systems are provided that comprise various improved sealing elements and features that enable stable storage of multiple syringes each comprising a liquid component or formulation prior to mixing.

In various embodiments, mixing syringe systems are provided comprising a first syringe, a second syringe, and a syringe coupler or coupling device. The syringe coupler is contemplated as comprising a device that is operable to interconnect the first and second syringe and to segregate contents of the two syringes during shipping and storage, for example, and which provides a reliable seal or closure element to achieve segregation while also obviating the need for users to assemble the syringes prior to mixing. In some embodiments, a first syringe, a second syringe, and a syringe coupler are provided and the components are interconnected for shipping and storage such that a user need not assemble components prior to mixing for subsequent administration of a drug or a therapeutic agent.

While various embodiments of the present disclosure contemplate a first syringe comprising first syringe contents (e.g. fluid) and a second syringe comprising second syringe contents (e.g. solid(s)), further embodiments contemplate that a first syringe may be initially devoid of material and a second syringe is provided that comprises one or more contents that require mixing. Alternatively, embodiments are contemplated that comprise a first syringe and a second syringe wherein contents of both syringes comprise liquid(s) to be mixed with one another. It should be recognized that various storing, mixing, and reconstitution applications are contemplated by embodiments of the present disclosure and inventive aspects of the disclosure are not limited to any particular intended use or application.

In some embodiments, a user-activated element is provided with systems of the present disclosure. The user-activated element preferably comprises an irreversible “one-way” feature wherein the user is allowed to activate the element and move the element from a first state to a second state but cannot return the element to the first state. For example, in some embodiments, a valve activation element is provided that is operable to be moved by a user from a first position (e.g. a sealed position) to a second position (e.g. a flow-enabled position) but cannot perform a reverse operation to close the element (i.e. the operation from a first position to a second position is irreversible). Embodiments of the present disclosure provide features to prevent improper use of the system including, but not limited to, features to substantially prevent the system from returning to an initial position and thereby requiring administration and product use after mixing.

In various embodiments, a syringe coupler or hub member is provided wherein at least one syringe can be connected to the syringe coupler and wherein removal of the syringe from the coupler is prevented or impeded. In some embodiments, for example, a first syringe comprising a polymer is operable to be connected and/or disconnected from a syringe coupler prior to activation or adjustment of the syringe coupler. Once the syringe coupler has been activated (e.g. a fluid flow path in the coupler has been opened), the first syringe is no longer capable of being removed. Preventing removal of at least one syringe from the coupler is useful, for example, to guide the user in the use of a second syringe (and only a second syringe) during administration once mixing is completed and substantially prevent any other use of the system after activation and/or mixing.

In various embodiments, systems and devices of the present disclosure comprise an elastomer element operable to seal a fluid pathway between first and second syringes. It is contemplated that devices and systems of the present disclosure are suitable for housing various materials and agents including, but not limited to: drugs and other therapeutic agents (in solid, e.g. lyophilized, or semi-solid or liquid/fluid form); liquid or flowable diluents, excipients or solvent systems; solvent systems further comprising co-solvents; polymer-solvent systems; polymer-solvent systems comprising co-polymers; or any combination thereof. A non-limiting example of a solvent suitable for use in the systems and devices of the present disclosure is N-methyl-2-pyrrolidone (NMP), a liquid organic solvent with known volatility capable of producing gaseous vapor, which can circulate within enclosed spaces such as an inner plastic tray enclosure or outer carton commonly associated with a drug products packaging. While various embodiments of the present disclosure contemplate packaging and sealing elements with a secure seal for various materials and uses, some embodiments contemplate and provide systems that securely houses NMP, or similar solvent, and separates the NMP from a solid such as lyophilized leuprolide acetate. If NMP is allowed to migrate into a storage or housing area of the solid, the active pharmaceutical ingredient could be degraded such that shelf life and overall stability of the materials are reduced. It should be recognized that other materials and applications stand to benefit from storage and sealing elements that provide a secure seal (at least prior to mixing) and the present disclosure is not limited to NMP and/or leuprolide acetate. Embodiments of the present disclosure as shown and described herein provide for reliable physical separation and prevention of migration of vapor, liquids, and solids.

Various elastomers (e.g. Santoprene, silicone, and other pharmaceutically acceptable thermoplastics as will be recognized by one of ordinary skill in the art) are contemplated for use with valve assemblies of embodiments of the present disclosure to inhibit or prevent liquid or gaseous solvent transmission. In further embodiments, it is contemplated that plastic components (including plastic-on-plastic components) are provided that create sufficient sealing to prevent liquid or gaseous solvent transmission between syringes and other components of the present disclosure.

While various materials are contemplated for use with embodiments of the present disclosure, preferred embodiments of the present disclosure contemplate the use of materials that are operable to withstand and are compatible with terminal manufacturing sterilization using, e.g., e-beam irradiation, gamma radiation, x-ray techniques, and ethylene oxide sterilization.

In one embodiment, a syringe-to-syringe mixing system is provided that comprises a first syringe barrel comprising a hollow body defining an internal chamber, and the hollow body has a proximal end and a distal dispensing end with an outlet. The system also comprises a second syringe barrel comprising a hollow body defining an internal chamber, the hollow body of the second syringe barrel having a proximal end and a distal dispensing end with an outlet. The first syringe barrel and the second syringe barrel each comprise a plunger slidably disposed within the syringe barrel for applying pressure to a content housed within the internal chambers. A syringe coupler is provided that is operable to receive the first syringe barrel and the second syringe barrel. The syringe coupler comprises a displaceable seal that comprises a flow port that is offset from the outlet of at least one of the first syringe barrel and the second syringe barrel when the displaceable seal is provided in a first position, and wherein the flow port is aligned with the outlet of the first syringe barrel and the second syringe barrel when the displaceable seal is provided in a second position.

In another embodiment, a syringe-to-syringe mixing system is provided that comprises a first syringe barrel with a hollow body, the hollow body having a proximal end and a distal dispensing end with an outlet; and a second syringe barrel with a hollow body and an outlet, the second syringe comprising a distal dispensing end with an outlet. The first syringe barrel and the second syringe barrel each comprise a plunger slidably disposed within the syringe barrel for applying pressure to a material housed within the internal chambers. A valve assembly is provided that is operable to receive the first syringe barrel and the second syringe barrel. The valve assembly comprises a displaceable user-interface operable to receive a force from a user and transmit the force to a displaceable seal, and wherein the displaceable user-interface is moveable in a direction substantially perpendicular to a longitudinal axis of at least one of the first syringe barrel and the second syringe barrel.

In various embodiments, methods of mixing syringe contents and preparing therapeutic agents are provided. In one embodiment, a method of preparing a therapeutic agent is provided that comprises providing a first syringe barrel and a second syringe barrel. The first syringe barrel and the second syringe barrel each comprise a plunger slidably disposed therein for applying pressure to a material housed within the syringe barrels. A syringe coupler is provided that is operable to receive the first syringe barrel and the second syringe barrel, and the syringe coupler comprises a user-interface and a displaceable seal. The method comprises securing at least one of the first syringe barrel and the second syringe barrel to the syringe coupler, applying a force to the user-interface to move the displaceable seal from a first position to a second position, and applying force to the plunger of the first syringe barrel and the second syringe barrel in an alternating manner to mix contents of the first syringe barrel and the second syringe barrel.

Also disclosed is a displaceable member of a syringe-to-syringe mixing system, for packaging and sealing of mixing syringes, the displaceable member comprising: an elastomeric member comprising a first side and an opposing second side; the first side and the second side each comprising a planar portion; at least one aperture provided through the elastomeric member and forming a fluid flow path through the elastomeric member; the first side of the elastomeric member comprising a raised projection with a first portion that surrounds the at least one aperture; and wherein the raised projection is at least partially surrounded by the planar portion.

In some embodiments, the syringe-to-syringe mixing system comprises: a first syringe comprising a hollow body, the hollow body having a proximal end and a distal dispensing end; a second syringe comprising a hollow body, the second syringe comprising a distal dispensing end; the first syringe and the second syringe each comprising a barrel and a plunger for applying pressure to a syringe content; a valve assembly that is operable to receive the first syringe and the second syringe, and wherein the valve assembly comprises at least one resilient member that is biased toward a locked position; wherein the valve assembly comprises a displaceable member comprising a user-interface and a guide member, wherein the displaceable member is slidable relative to the guide member, and wherein the user-interface is operable to receive a force from a user and transmit the force to a displaceable seal provided within the displaceable member; wherein the displaceable seal comprises a planar portion, a protrusion, and a flow path, and wherein the displaceable seal is moveable in a direction substantially perpendicular to a longitudinal axis of at least one of the first syringe and the second syringe; a selectively rotatable member operable to receive at least one of the first syringe and the second syringe; wherein the valve assembly comprises a first position wherein a fluid flow between the first syringe and the second syringe through the displaceable seal is fully occluded, and a second position in which fluid is allowed to flow through the displaceable seal and between the first syringe and the second syringe; wherein the displaceable member is provided in communication with the selectively rotatable member when the displaceable member is in the first position, and wherein the displaceable member is displaced to a position that allows rotation of the selectively rotatable member when the displaceable member is in the second position.

In additional embodiments, the syringe-to-syringe mixing system comprises: a combined syringe coupler and valve assembly that is operable to receive a first syringe and a second syringe; wherein the valve assembly comprises a first portion and a second portion that are displaceable relative to one another between at least a first position and a second position; the first portion and the second portion each comprising a syringe receiving portion and an internal flow port; wherein the second portion comprises a sealing element that is displaceable with and in fixed relative position to the second syringe; the sealing element comprising a flow path and at least one of a projection and an upstanding portion positioned circumferentially around a central axis of the flow path, and wherein the sealing element is moveable between the first position and the second position, the first position comprising a sealed position and the second position comprising a mixing position, wherein in the first position, the internal flow port of the second portion is offset from the internal flow port of the first portion, and wherein in the second position, the internal flow port of the second portion is aligned or substantially aligned with and in fluid communication with the internal flow port of the first portion; and wherein with the sealing element in the first position, the at least one of a projection and the upstanding portion provides a contact seal against the first portion to prevent leakage of fluid from the internal flow port of the first portion to the internal flow port of the second portion and to prevent leakage of fluid from the internal flow port of the second portion to the internal flow port of the first portion, and wherein with the sealing element in the second position, the at least one of a projection and the upstanding portion provides a contact seal against the first portion that surrounds the internal flow port of the first portion to prevent leakage of fluid passing between the internal flow ports of the first and second portions to allow mixing.

Also described is a syringe coupler configured to couple to a first syringe and a second syringe, each of the first and second syringes having a respective male connector, the syringe coupler comprising: a guide member defining a syringe receiving portion, the syringe receiving portion defining an internal flow port, wherein the guide member is configured to engage the male connector of the second syringe to establish fluid communication between the internal flow port of the syringe receiving portion and the second syringe; a displaceable member axially slidable relative to the guide member about and between a first position and a second position; and a syringe engagement member at least partly housed within the guide member, the syringe engagement member defining an internal flow port, wherein the syringe engagement member is configured to engage the male connector of the first syringe to establish fluid communication between the internal flow port of the syringe engagement member and the first syringe; and a sealing element defining a flow port and at least one of a projection and an upstanding portion positioned circumferentially around a central axis of the flow port, wherein the sealing element is coupled to and in a fixed position relative to the displaceable member, wherein: when the displaceable member is in the first position, the internal flow port of the syringe receiving portion of the displaceable member is offset from the internal flow port of the syringe engagement member, and the at least one of the projection or the upstanding portion of the sealing element forms a contact seal with the syringe engagement member that prevents leakage of fluid from the internal flow port of the syringe receiving portion of the displaceable member to the internal flow port of the syringe engagement member and prevents leakage of fluid from the internal flow port of the syringe engagement member to the internal flow port of the syringe receiving portion of the displaceable member; and when the displaceable member is in the second position, the internal flow ports of the syringe receiving portion and the syringe engagement member and the flow port of the sealing element are aligned or substantially aligned to define a flow path, and the at least one of the projection or the upstanding portion of the sealing element provides a contact seal with the syringe engagement member that surrounds the internal flow port of the syringe engagement member to prevent leakage of fluid passing between the internal flow ports of the syringe receiving portion of the displaceable member and the syringe engagement member.

Also described are both solid-liquid and liquid-liquid formulations which can be contained in any of the above syringe systems including any of the described seals and syringe couplers. Also described are kits and uses of the pre-filled syringe systems. Also described are methods of treating patients using the pre-filled syringe systems, including for instance methods of treating prostate cancer, breast cancer, central precocious puberty, and other diseases and disorders.

The Summary is neither intended nor should it be construed as being representative of the full extent and scope of the present disclosure. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 is a perspective view of a mixing syringe system.

FIG. 2A is a cut-away perspective view of a component of a mixing syringe system according to one embodiment of the present disclosure.

FIG. 2B is a cut-away perspective view of a component of a mixing syringe system according to one embodiment of the present disclosure.

FIG. 3 is an exploded perspective view of a mixing syringe system according to one embodiment of the present disclosure.

FIG. 4A is a perspective view of a component of a mixing syringe system according to one embodiment of the present disclosure.

FIG. 4B is a perspective view of a component of a mixing syringe system according to one embodiment of the present disclosure.

FIG. 4C is a perspective view of a component of a mixing syringe system according to one embodiment of the present disclosure.

FIG. 5A is a perspective view of a component of a mixing syringe system according to one embodiment of the present disclosure.

FIG. 5B is a perspective view of a component of a mixing syringe system according to one embodiment of the present disclosure.

FIG. 5C is a perspective view of a component of a mixing syringe system according to one embodiment of the present disclosure.

FIG. 5D is a perspective view of a component of a mixing syringe system according to one embodiment of the present disclosure.

FIG. 6A is a perspective view of a component of a mixing syringe system according to one embodiment of the present disclosure.

FIG. 6B is a perspective view of a component of a mixing syringe system according to one embodiment of the present disclosure.

FIG. 7A is a cross-sectional elevation view of a mixing syringe system according to one embodiment of the present disclosure.

FIG. 7B is a cross-sectional elevation view of a mixing syringe system according to one embodiment of the present disclosure.

FIG. 8 is an elevation view of components of a mixing syringe system according to an embodiment of the present disclosure.

FIG. 9 is an elevation view of components of a mixing syringe system according to an embodiment of the present disclosure.

FIG. 10 is perspective view of a mixing system and associated packaging according to an embodiment of the present disclosure.

FIG. 11A is a cross-sectional elevation view of a component of a syringe mixing system according to one embodiment of the present disclosure.

FIG. 11B is a cross-sectional elevation view of the component of FIG. 11A in a second position.

FIG. 12A is a cross-sectional elevation view of a component of a syringe mixing system according to one embodiment of the present disclosure.

FIG. 12B is a cross-sectional elevation view of the component of FIG. 12A in a second position.

FIG. 12C is a side view of a component of a syringe mixing system according to one embodiment of the present disclosure.

FIG. 12D is a front view of the component of the embodiment of FIG. 12C.

FIG. 12E provides front and side elevation views of the component of the embodiment of FIG. 12C.

FIG. 12F provides front and side elevation views of the component of the embodiment of FIG. 12C.

FIG. 13 is an exploded view of a syringe mixing system according to one embodiment of the present disclosure.

FIG. 14A is a perspective view of a component of a mixing syringe system according to one embodiment of the present disclosure.

FIG. 14B is a perspective view of a component of a mixing syringe system according to one embodiment of the present disclosure.

FIG. 14C is a perspective view of a component of a mixing syringe system according to one embodiment of the present disclosure.

FIG. 15A is a perspective view of a component of a mixing syringe system according to one embodiment of the present disclosure.

FIG. 15B is a perspective view of a component of a mixing syringe system according to one embodiment of the present disclosure.

FIG. 15C is a perspective view of a component of a mixing syringe system according to one embodiment of the present disclosure.

FIG. 15D is a perspective view of a component of a mixing syringe system according to one embodiment of the present disclosure.

FIG. 16A is a perspective view of a component of a mixing syringe system according to one embodiment of the present disclosure.

FIG. 16B is a perspective view of a component of a mixing syringe system according to one embodiment of the present disclosure.

FIG. 17A is a cross-sectional elevation view of a mixing syringe system according to one embodiment of the present disclosure.

FIG. 17B is a cross-sectional elevation view of a mixing syringe system according to one embodiment of the present disclosure.

FIG. 18A is an elevation view of a component of a mixing syringe system according to one embodiment of the present disclosure.

FIG. 18B is an elevation view of the component of FIG. 18A.

FIG. 19A is a perspective view of a component of a mixing syringe system according to one embodiment of the present disclosure.

FIG. 19B is a perspective view of the component of FIG. 19A.

FIG. 19C is a perspective view of the component of FIG. 19A.

FIG. 20 is a perspective view of a component of a mixing syringe system in an assembled and an unassembled state.

FIG. 21 is a perspective view of a component of a mixing syringe system in an unassembled state.

FIG. 22A is a perspective view of a component of a mixing syringe system according to one embodiment of the present disclosure.

FIG. 22B is a cross-sectional elevation view of a component of a mixing syringe system in a first position and according to an embodiment of the present disclosure.

FIG. 23A is a cross-sectional elevation view of a component of a mixing syringe system in a first position and according to an embodiment of the present disclosure.

FIG. 23B is a cross-sectional elevation view of a component of a mixing syringe system in a second position and according to an embodiment of the present disclosure.

FIG. 24 is a perspective view of a component of a mixing syringe system according to one embodiment of the present disclosure.

FIG. 25A is a cross-sectional elevation view of a component of a mixing syringe system in a first position and according to an embodiment of the present disclosure.

FIG. 25B is a cross-sectional elevation view of a component of a mixing syringe system in a second position and according to an embodiment of the present disclosure.

FIG. 26 is a perspective view of first and second components of a mixing syringe system.

FIG. 27 is a side elevation view of a mixing syringe system according to one embodiment of the present disclosure.

FIG. 28 is a perspective view of a syringe coupler of the mixing syringe system of FIG. 27.

FIG. 29 is an elevation view of the syringe coupler of FIG. 28 in a first position.

FIG. 30 is a cross-sectional view of the syringe coupler of FIG. 28 in the first position.

FIG. 31 is an elevation view of the syringe coupler of FIG. 28 in a second position.

FIG. 32 is a cross-sectional view of the syringe coupler of FIG. 28 in the second position.

FIG. 33 is an exploded view of the syringe coupler of FIG. 28.

FIG. 34 is another exploded view of the syringe coupler of FIG. 28.

FIG. 35A is a cross-sectional view of a syringe coupler having a syringe engagement member that includes a dividing structure, with said syringe coupler shown in a first position. FIG. 35B is an elevation view of the syringe coupler of FIG. 35A in the first position. FIG. 35C is a cross-sectional view of the syringe coupler of FIG. 35A in a second position. FIG. 35D is an elevation view of the syringe coupler of FIG. 35A in the second position. FIG. 35E is a close-up view of FIG. 35D, showing additional detail of the dividing structure. FIG. 35F is a further close-up view of FIG. 35D, showing the divided structure in the open position. FIG. 35G shows the same structure in the closed position. Additional close-up images in which the divided structure is in the closed position are shown in FIGS. 35H and 35I.

FIG. 36A shows PK study blood plasma LA concentration results comparing liquid-solid and liquid-liquid 7.5 mg formulations.

FIG. 36B shows PK study blood plasma LA concentration results comparing liquid-solid, liquid-liquid 7.5 mg, and liquid-liquid 20% LA formulations.

FIG. 37 shows PK study blood serum LA concentration results comparing liquid-solid to liquid-liquid 22.5 mg formulations.

FIG. 38 shows PK study blood plasma LA concentration results comparing liquid-solid formulation to liquid-liquid 45 mg formulation and liquid-liquid formulations with additional solvent; Test Article 3 (244 mg NMP) and Test Article 4 (259 mg NMP).

FIG. 39 shows in vitro data comparing the average cumulative percent release of LA from the polymer depot for liquid-solid and liquid-liquid 7.5 mg formulations.

FIG. 40 shows in vitro data comparing the average cumulative percent release of LA from the polymer depot for liquid-solid and liquid-liquid 22.5 mg formulations.

FIG. 41 shows in vitro data comparing the average cumulative percent release of LA from the polymer depot for liquid-solid and liquid-liquid 30 mg formulations.

FIG. 42 shows in vitro data comparing the average cumulative percent release of LA from the polymer depot for liquid-solid and liquid-liquid 45 mg formulations.

FIG. 43 shows in vitro data from a design of experiments (DOE) study showing the gelation threshold concentration for leuprolide acetate and solvent in a drug solution.

FIG. 44A is a plot of viscosity versus polymer solution composition (wt % in NMP) for a 50:50 PLG polymer.

FIG. 44B is a plot of viscosity versus polymer solution composition (wt % in NMP) for a 75:25 PLG polymer.

FIG. 44C is a plot of viscosity versus polymer solution composition (wt % in NMP) for a 85:15 PLG polymer.

FIG. 45 is a plot of LA content versus e-beam dose for 34% LA in NMP as compared to solid LA.

FIG. 46 is a plot comparing mixing efficiency for a liquid-liquid formulation vs. a solid-liquid formulation at various dose strengths.

FIG. 47 is a plot of in-vitro release testing comparisons of a liquid-liquid formulation which demonstrates % release vs. mixing cycles.

FIG. 48 is a plot of CP Assay versus PS:DS Fill Ratio.

### DETAILED DESCRIPTION

### A. Syringe-to-Syringe Mixing Systems

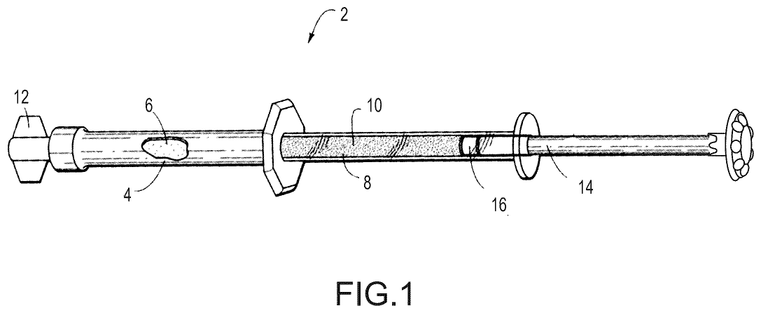

FIG. 1 is a perspective view of a syringe-to-syringe mixing system. As shown, the system 2 comprises a first syringe 4 housing contents 6 and a second syringe 8 housing contents 10. The syringes 4, 8 are connected at their respective distal dispensing ends. Fluid and materials housed within the syringes may be moved from one syringe to another and mixing can occur by applying forces to the syringe plunger rods 12, 14. At least one plunger 16 is operable to force contents between the syringes 4, 8 and produce a mixing action. Various known syringes and systems require the first syringe and second syringe to be directly connected (e.g. threaded together) by a user just prior to mixing and administration. The syringes are then disconnected with one syringe comprising a mixed solution for administration.

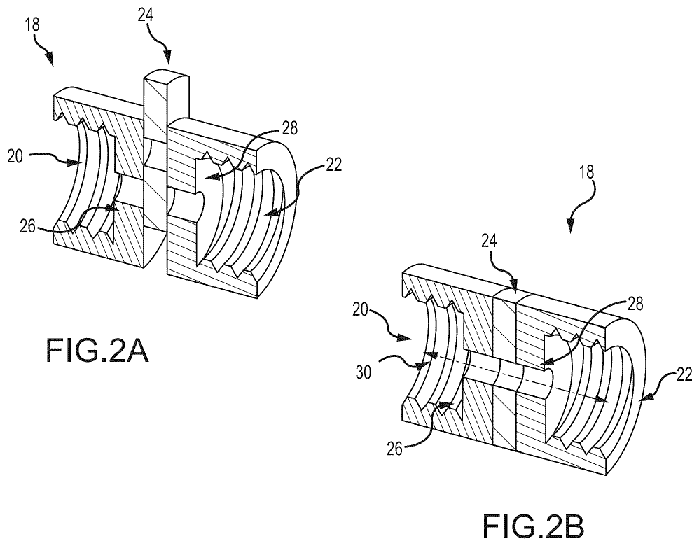

FIGS. 2A-2B show perspective views of a component of a valve assembly for a mixing system contemplated for use with syringes according to one embodiment of the present disclosure. As shown, a coupling element is provided as a syringe coupler 18. The syringe coupler 18 is operable to receive a first and second syringe and selectively provide the syringes in fluid communication with one another. The syringe coupler 18 comprises a first end 20 operable to receive a first syringe and a second end 22 operable to receive a second syringe. The syringes (not shown in FIGS. 2A-2B) are contemplated as comprising distal ends with open outlets for dispensing and/or receiving materials. FIGS. 2A-2B illustrate the first and second ends 20, 22 as comprising female threaded connection members. It will be recognized, however, that syringe couplers of the present disclosure are not limited to threaded connections and one or both of the first and second ends may comprise alternative structures for receiving and securing syringes. As further shown in FIGS. 2A-2B, the syringe coupler 18 comprise a valve element comprising a displaceable member 24 that is moveable relative to the coupler 18 in a direction substantially perpendicular to a longitudinal axis of the syringe coupler. First and second internal members 26, 28 are provided that each comprise an aperture and which cooperate with the displaceable member.

As shown in FIG. 2A, a closed position is provided wherein the displaceable member 24 is in a first position and a central aperture of the displaceable member is offset from the apertures of the first and second internal members 26, 28. In this position, fluid and gaseous vapor flow is at least partially and preferably fully occluded through the valve assembly of the coupler. Accordingly, syringes connected to the coupler 18 cannot exchange materials in the closed position. The displaceable member 24 is offset and preferably comprises a surface or user-interface that is accessible to a user and operable to receive an activation force from the user to a displaceable seal within the displaceable member.

An activation force upon the displaceable member 24 is operable to move the displaceable member from a first position (or closed position) (FIG. 2A) wherein fluid flow through the member 18 is occluded to a second position (or open position) (FIG. 2B) wherein an aperture of the displaceable member 24 is aligned with apertures of the internal members 26, 28 and a fluid flow path 30 is formed through the valve assembly of the device. As shown and described the syringe coupler 18 provides means for securing at least one and preferably two syringes, and comprises a valve assembly to selectively allow for transmission of materials between syringes upon activation of the valve assembly by a user.

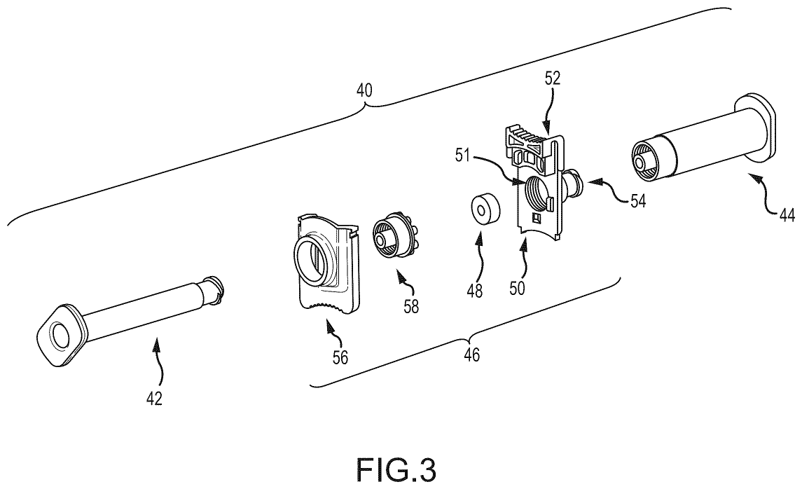

FIG. 3 is an exploded perspective view of a syringe-to-syringe mixing system 40 according to another embodiment of the present disclosure. As shown, the system 40 comprises a first syringe 42 and a second syringe 44. The first and second syringes are contemplated as initially comprising solid or liquid contents. For example, the first syringe 42 may comprise a polymer-solvent system such as, but not limited to, a biodegradable polymer dissolved in NMP and the second syringe 44 may comprise a drug lyophilizate such as, but not limited to, lyophilized leuprolide acetate. Although the discussion of various embodiments of the present disclosure contemplates and refers to the first syringe comprising NMP and the second syringe comprising a drug lyophilizate, it will be recognized that the disclosure is not limited to that arrangement. Syringe contents may be altered, rearranged and substituted while remaining within the scope of the inventions of the present disclosure. Indeed, inventive aspects of the present disclosure are believed to reside in features and components of the described system regardless of which materials (or if any materials) are provided within the components.

Unwanted NMP migration (i.e. unintended migration prior to mixing) has been recognized as providing various complications including, for example, degrading or destroying shelf-life of contents. It is an object of various embodiments of the present disclosure to reduce or eliminate the risks of unwanted NMP migration while storing NMP and a drug lyophilizate in close proximity prior to mixing.

The contents of the first and second syringes 42, 44 may be mixed to formulate a solution or suspension for administration as shown and described herein. The embodiment of FIG. 3 comprises a combined syringe coupler and valve assembly 46. The combined syringe coupler and valve assembly 46 of the depicted embodiment is operable to receive and connect to the first and second syringes 42, 44, selectively prevent and enable fluid transfer between the two syringes, and selectively prevent removal of at least one syringe.

Each syringe 42, 44 comprises a barrel having an internal volume defined by a hollow body, proximal ends for receiving a plunger for applying pressure to a syringe content (not shown in FIG. 3), and distal ends with dispensing outlets wherein the distal ends are operable to connect to the combined syringe coupler and valve assembly 46. The combined syringe coupler and valve assembly 46 comprises a valve assembly with a elastomeric element 48 that nests within recessed area 51 of a displaceable member 50. In some embodiments, including that shown in FIG. 3, the displaceable member comprises an annular sealing element. The sealing element 48 can have an elastomeric member comprising a first side and an opposing second side, the first and second sides each comprising a planar portion. An aperture can be provided through the elastomeric member which forms a fluid flow path therethrough. In addition, the first and/or second sides of the elastomeric member can have a raised projection with a first/second portion that surrounds the at least one aperture, where the raised projection(s) are at least partially surrounded by the planar portion.

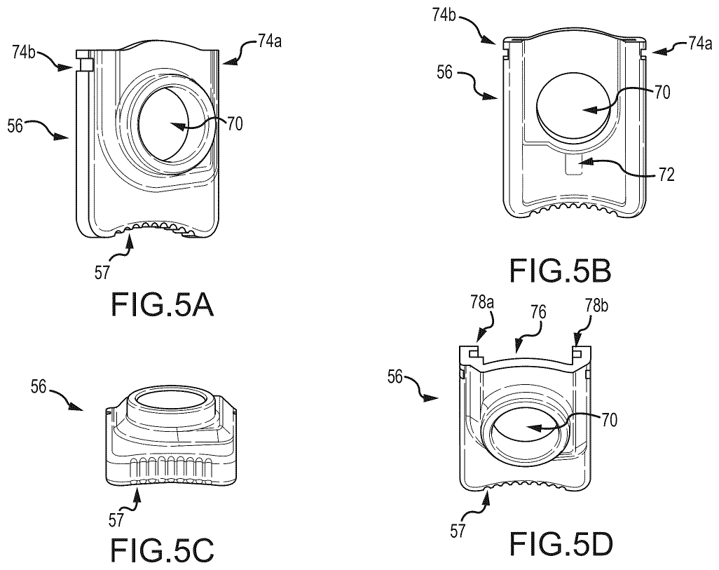

The displaceable member of the valve assembly comprises a user-interface 52 that is operable to be contacted by and receive a force from a user and a male extension 54 for receiving the second syringe 44. The displaceable member in the valve assembly of the syringe coupler 46 further comprises a guide member 56. The displaceable member comprises a user interface 57 (FIG. 5A) that is operable to be contacted by and receive a force from a user and transmit the force to a displaceable seal provided within the displaceable member. A rotatable Luer lock member 58 is provided. The rotatable Luer lock member 58 of the depicted embodiment comprises a proximal end with a male fitting operable to connect to the first syringe 42, and a distal end comprising a cog with teeth or projections for selectively limiting rotation of the rotatable Luer lock member 58 prior to activation.

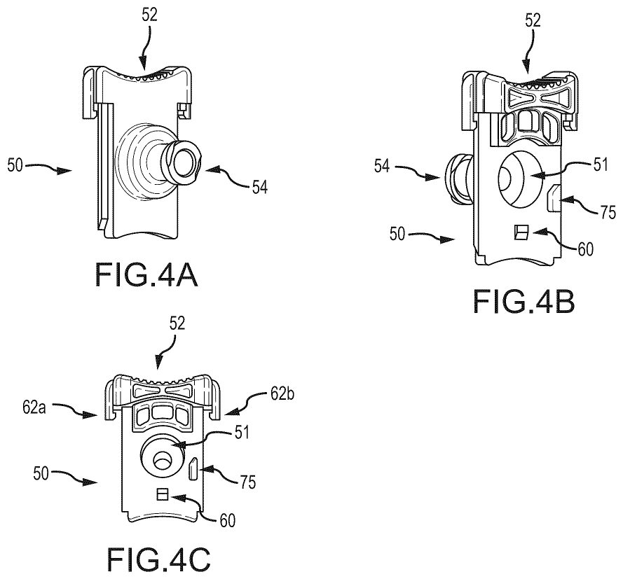

FIGS. 4A-4C are perspective views showing the displaceable member 50 in greater detail. As shown, the displaceable member 50 comprises a user interface 52 operable to be acted upon by a user and also comprises a guide member. In embodiments, the displaceable member is displaceable in a downward direction (at least relative to FIG. 4A) and is preferably not operable to return to an initial or first position, although the valve assembly comprising the displaceable member can be biased toward a locked position. A male extension 54 is provided on one side of the member for receiving a syringe. A recess 51 is provided on an opposing side of the displaceable member relative to the male extension 54. The recess 51 is operable to receive a sealing element, such as the annular sealing element 48 of FIG. 3. A channel is provided through the displaceable member 50, wherein the channel extends through the male extension 54 and into the recess 51. In some embodiments, the sealing element comprises an aperture through an elastomeric member of the sealing element that is aligned with the channel of the displaceable member 50.

As shown in FIGS. 4A-4B, first and second raised projections 60, 75 are provided on the displaceable member 50. The projections 60, 75 are displaceable with the member 50 and are moveable relative to at least the rotatable Luer lock member 58 of an assembled device. In a first position, at least one of the projections is provided in contact with the rotatable Luer lock member 58 to prevent rotation of the member 58. This contact and related locking of the rotatable Luer lock member 58 enables a first syringe to be threaded onto (and threadably removed from) the rotatable Luer lock member 58 prior to activation of the assembled device. Movement of the displaceable member 50 by user activation results in displacing the projections 60, 75 such that they are not in contact with the Luer lock member 58. With rotation of the rotatable Luer lock member enabled, the member 58 is free to spin within the displaceable member 50. Without resistance, a syringe connected to the rotatable Luer lock member 58 is prevented from being threadably detached from the syringe coupler even if and when a rotation is applied in an attempt to remove the syringe. It is an object of the present disclosure to provide a syringe coupler 46 that retains at least one syringe such that a user is prevented from using the first syringe for administration and is thereby only given the option of administering the mixed contents with the second syringe.

As shown in FIGS. 4A-4C, the displaceable member 50 also comprises clips or resilient projections 62a, 62b. The resilient projections 62a, 62b are operable to flex outwardly and do not substantially impede a downward movement of the displaceable member 50. When provided in a second position, however, the resilient projections 62a, 62b are secured to the guide member 56 at least in part due to an inherent restoring force of the projections. The resilient projections 62a, 62b secure the displaceable member 50 in a second position within guide member 56 through engagement of said resilient projections 62a, 62b into recesses 74a, 74b located upon guide member 56 to prevent or inhibit the displaceable member from being returned to a first position.

FIGS. 5A-5D are perspective views of a guide member 56 of the displaceable member according to one embodiment and contemplated for use and cooperation with the displaceable member 50 is FIGS. 4A-4C. As shown, the guide member 56 comprises a central aperture 70 to permit fluid flow and to receive a rotatable Luer lock member 58 of embodiments of the disclosure. The guide member 56 is provided to slidably receive at least a portion of a displaceable member 50. As shown, the guide member 56 comprises a receiving portion 76 with first and second slot members 78a, 78b to receive a displaceable member 50. The displaceable member having the guide member comprises a user-interface 57 that is operable to be contacted by and receive a force from a user. The guide member comprises a user-interface 57 that is operable to be contacted by and receive a force from a user. In certain embodiments, and as shown in FIGS. 5A-5D, the user-interface 57 is contemplated as comprising a gripping or contact surface having ridges to reduce slipping and provide ergonomic benefits.

A surface of the guide member 56 comprises a channel 72 (FIG. 5B) to receive and guide the movement of a ramp-like (or raised) projection 60 of the displaceable member 50 (FIG. 4B, for example). During assembly of the syringe-to-syringe mixing system, the guide member 56 operably receives the displaceable member 50 such that raised projection 60 of the displaceable member 50 contacts an upper surface of receiving portion 76 of the guide member 56 (at least relative to the direction in FIG. 5B) to induce a physical separation between the surface of the displaceable member 50 and the receiving portion 76 of the guide member 76. The ramp-like (or raised) projection 60 allows a distal surface of the annular sealing element 48 nesting within recessed area 51 of the displaceable member 50 to slide over the distal surface of Luer lock member 58 nested within the central aperture 70 of guide member 56. Once ramp-like (or raised) projection 60 traverses the distal surface of Luer lock member 58 nested within the central aperture 70 of guide member 56, the raised projection 60 is operably received into one of the plurality of teeth 86 of the Luer lock member 58. Operable engagement of ramp-like (or raised) projection 60 into one of the plurality of teeth 86 of the Luer lock member 58 collapses the physical separation induced during crossing of the distal surface of Luer lock member 58 such that the distal surface of the annular sealing element 48 nested within recessed area 51 of the displaceable member 50 is brought into direct contact with the distal surface of Luer lock member 58 within the central aperture 70 of guide member 56 causing the annular sealing element 48 to compress. Once ramp-like (or raised) projection 60 is operably received into one of the plurality of teeth 86 of the Luer lock member 58 and annular sealing element 48 is compressed, the assembled syringe device connector is configured into the first position prior to activation. The ramp-like projection 60 is operable to allow the guide member 56 to translate over various irregular surface including, for example, a central aperture of the Luer lock member 58.

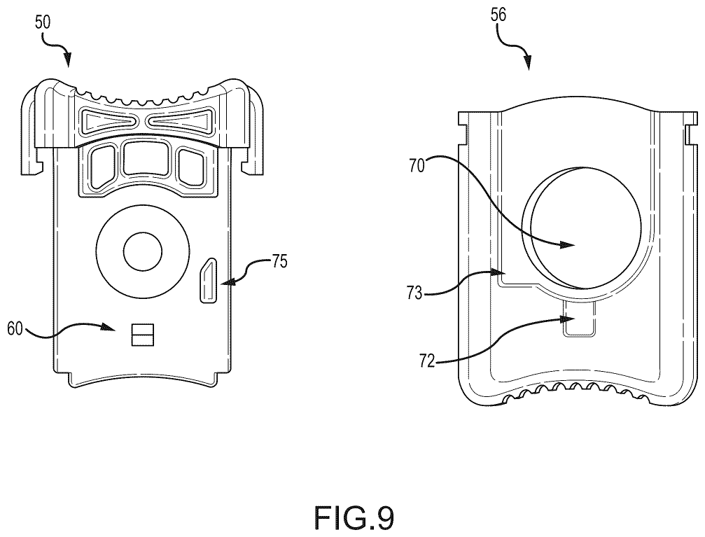

In a first position prior to activation, the ramp-like (or raised) projection 60 and the projection 75 of the displaceable member 50 are provided in communication with a rotatable Luer lock member 58 to prevent rotation thereof. In a second position subsequent to activation, the projection 60 of the displaceable member 50 is displaced into the channel 72 of guide member 56 while projection 75 of the displaceable member is displaced into the recessed area 73 (FIG. 9) on the guide member 56 of the displaceable member where the projections 60, 75 assume positions that do not contact or impede rotation of the rotatable Luer lock member 58. The second position further comprises a position wherein a fluid flow channel is created. Specifically, an annular sealing element 48 provided within the displaceable member 50 is moved from a first position characterized by a channel of the annular sealing element 48 being offset from and preventing flow between inlets and outlets of interconnected syringes and a second position characterized by the channel of the annular sealing element 48 being provided in axial alignment with the syringe outlets and inlets.

As shown in FIGS. 5A-5B, for example, the guide member 56 further comprises recesses 74a, 74b that are operable to receive resilient projections 62a, 62b of a displaceable member and secure the syringe coupler in a second position. The recesses 74a, 74b are operable to prevent or at least impede a user from returning the device to a first position after activation of the syringe coupler.

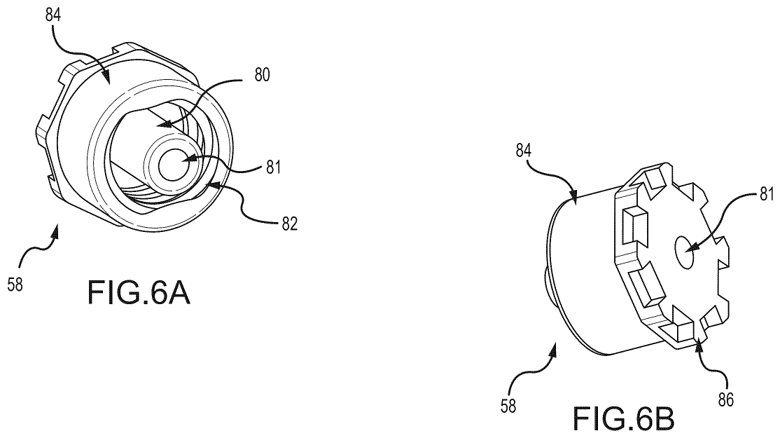

FIGS. 6A-6B are perspective views of a rotatable Luer lock member 58 according to one embodiment of the present disclosure. As shown, the rotatable Luer lock member 58 comprises a first end with a male Luer lock 80 that provides a means of attachment to a first syringe as well as a fluid flow path through a central aperture 81 of the member 58. The male Luer lock 80 is at least partially provided within a threaded female member 82 that is operable to threadingly engage a first syringe. A bearing surface 84 is provided on an exterior of the member 58. The bearing surface 84 is operable to be provided in the central aperture 70 of the guide member 56 and contact the guide member. The bearing surface 84 of the rotatable Luer lock member 58 comprises a surface upon which the member 58 can rotate (when unlocked) and contact the central aperture 70 of guide member 56. The rotatable Luer lock member 58 further comprises a plurality of teeth 86 operable to act as locking members and selectively prevent rotation of the rotatable Luer lock member 58. Specifically, when a projection of the present disclosure (75 of FIG. 4B, for example) is provided in a first position, the projection 75 is provided in contact with at least one of the plurality of teeth 86 such that rotation of the rotatable Luer lock member 58 (at least with respect to the guide member 56 and the displaceable member 50) is prevented. The secured nature or state of the member 58 in the first position allows a user to thread a first syringe within the threaded female member 82. When the displaceable member is displaced as shown and described herein, the projection 75 is moved away from the plurality of teeth 86 of the rotatable Luer lock member 58 such that rotation is unopposed and the member 58 is allowed to rotate relative to the guide member 56 and the displaceable member. This freedom of rotation prevents or at least inhibits the un-threading and removal of the first syringe as a rotation force applied to the syringe will cause a rotation of the rotatable Luer lock member 58. With no significant oppositional force on the rotatable Luer lock member 58 or threaded female member 82, un-threading will not occur and the first syringe is effectively prevented from being removed from the syringe coupler.

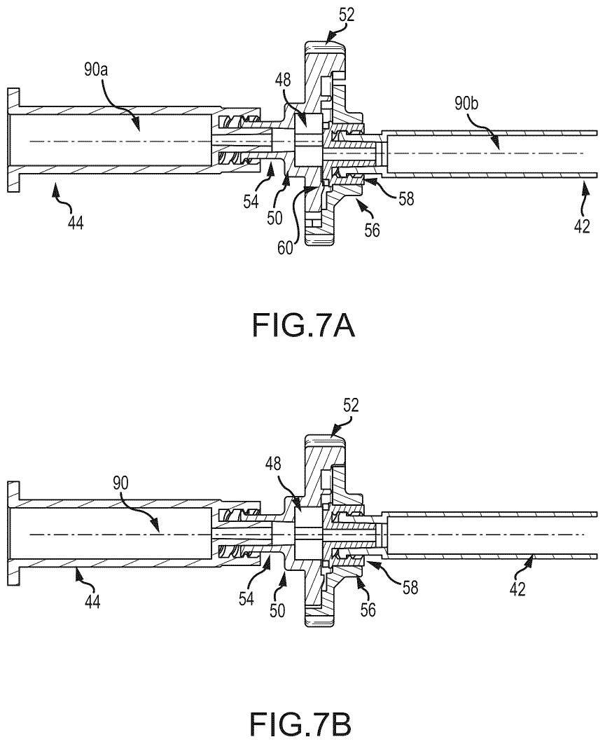

FIGS. 7A-7B are cross-sectional elevation views of a system according to an embodiment of the present disclosure. As shown and previously described, the system comprises a first syringe 42 and a second syringe 44. The syringes 42, 44 are connected to a syringe coupler and valve assembly comprising a displaceable member 50 with a user-interface 52, an annular sealing element 48, a guide member 56, and a rotatable Luer lock member 58 provided at least partially within the guide member 56. The system is shown as being provided in a first position in FIG. 7A. The first position comprises a position wherein the displaceable member and associated annular sealing element 48 are provided offset from a central axis and passageway of the rotatable Luer lock member 58. Specifically a fluid flow path 90a of the second syringe 44, the male extension 54 of the displaceable member 50, and the sealing member 48 is offset from and not in communication with a fluid flow path 90b of the first syringe 42 and the rotatable Luer lock member 58. Fluid and gaseous vapor flow between syringes 42, 44 is thus prevented.

FIG. 7B depicts the system in a second position wherein the displaceable member 50 has been displaced by application of force upon the user-interface 52 (for example). As shown, fluid pathway 90a of FIG. 7A and related components have been displaced such that a continuous fluid pathway 90 is provided and fluid flow between the first syringe 42 and second syringe 44 is enabled. Mixing of contents is thus enabled, wherein plunger rods (not shown in FIGS. 7A-7B) associated with the first and second syringes 42, 44 are operable to force contents between the syringes.

Systems, devices and methods of the present disclosure are not limited to any particular therapeutic agent(s), solution(s), suspension(s), gas, or a combination thereof. In some embodiments, for example, it is contemplated that that one or more non-lyophilized materials are provided in syringes of the present disclosure. In some embodiments, a gas (e.g. Cobalt gas) is provided in a syringe for mixing with contents of a second syringe. Such embodiments, including others, complete that mixing syringe systems of the present disclosure comprise gas-impermeable materials to prevent gas permeation and migration. However, in certain preferred embodiments, a first syringe 42 is initially provided with a liquid formulation component such as a polymer-solvent system and a second syringe is provided with an API, which may, in some non-limiting instances, be present as a lyophilized powder. In such embodiments, the contents are stored separately with each respective syringe which are interconnected to the syringe coupler with the displaceable member provided in the first position (FIG. 7A). To administer a therapeutic agent, the displaceable member is depressed or otherwise activated, creating the fluid-flow pathway 90 of FIG. 7B. Repetitive mixing may then be performed by forcing the polymer-solvent of the first syringe 42 into the second syringe that comprises the API, forcing the contents back to the first syringe, and repeating the process until desired mixing is achieved. As discussed, the second position of FIG. 7B is characterized by the presence of a fluid flow path between the two syringes 42, 44, as well as by the disengagement of the displaceable member 50 and the rotatable Luer lock member 58. Specifically, the second position (FIG. 7B) comprises a position in which the rotatable Luer lock member 58 is free to rotate within the syringe coupler and the first syringe 42 is prevented from unthreading or detachment. Accordingly, the second syringe 44 preferably comprising the mixed or prepared agent is detachable for use as an injection syringe while the first syringe is inoperable for such purposes.

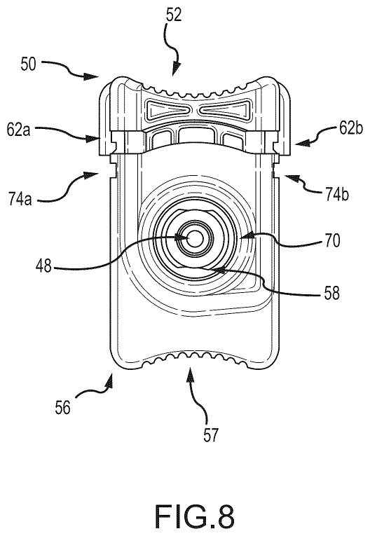

FIG. 8 is an elevation view of components of a syringe coupler and valve assembly according to an embodiment of the present disclosure. As shown, a displaceable member 50 and a guide member 56 are provided in a first position. The first position is suitable for shipping and storage wherein fluid and gas vapor flow between interconnected syringes is fully or at least partially occluded. The displaceable member 50 comprises an interconnected sealing element 48. The sealing element 48 comprises a central aperture, but the central aperture is offset from the fluid flow path of the guide member 56 and rotatable Luer lock member 58 such that fluid flow through the device is occluded. The displaceable member 50 and the guide member 56 comprise user-interface portions 52, 57, respectively. Force may be applied to one or more of the user-interfaces 52, 57 to convert the device from the first position to a second position wherein the displaceable member 50 is displaced relative to the guide member and a fluid flow path is created (FIG. 7B, for example).

The displaceable member 50 comprises first and second raised projections 62a, 62b that are operable to be outwardly displaced upon downward movement of the displaceable member. The first and second raised projections 62a, 62b are secured within the recesses 74a, 74b of the guide member 56 and move inwardly based on their inherent material properties and elasticity. The placement of the first and second projections 62a, 62b within or partially within the recesses 74a, 74b of the guide member 56 prevent or inhibit a return movement of the displaceable member 50 back to the first position.

FIG. 9 is an elevation view of cooperating surfaces of a displaceable member 50 and a guide member 56. As shown, the displaceable member 50 comprises a ramp-like (or raised) projection 60 that is operable to guide installation and interconnection of the displaceable member 50 and the guide member 56. A channel 72 is provided to receive and house the projection 60. In a first position, rotation of a rotatable Luer locking member is substantially impeded by a second projection 75 being provided in contact with a portion of the Luer locking member. In a second position, the second projection 75 is displaced downwardly (at least with respect to FIG. 9) and the rotatable Luer locking member can freely rotate within the aperture 70 of the guide member 56. The second projection 75 is provided on the displaceable member 50 and is operable to contact at least one of a plurality of teeth of a rotatable Luer lock member 58 (not shown) in a first position and which is disposed into slot 73 provided on the guide member 56 in a second position. In addition to locking and unlocking a rotatable Luer lock member 58 (not shown), the projection 75 of the displaceable member and the corresponding features of the guide member 56 serve to limit displacement of the displaceable member and ensure that the displaceable member comes to rest in a second position wherein a fluid flow path is aligned.

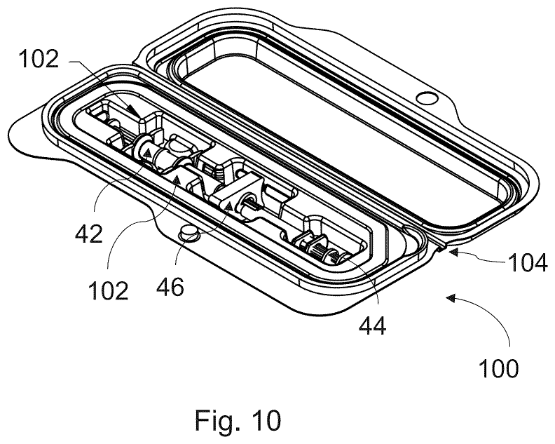

FIG. 10 is a perspective view of a syringe-to-syringe mixing system provided in packaging tray 100 according to one embodiment of the present disclosure. As shown, the system comprises a first and second syringe 42, 44 joined by a syringe coupler 46 including (for example) those shown and described herein. The syringes 42, 44 are connected to the syringe coupler 46 for and during shipping and storage in a packaging member 100. The packaging member 100 of FIG. 10 comprises a clamshell device rotatable about a hinge 104 and in which the system is stored. Contours and indentations 102 of the packaging member 100 are contemplated as being provided to restrict movement of certain components of the system including, for example, unwanted movement of a displaceable member (50 in FIG. 3, for example) and/or unwanted movement of syringe plunger rods. While various embodiments of the present disclosure contemplate the provision of first and second syringes attached to a syringe coupler for shipping and wherein the system is provided to an end user in an assembled or interconnected state, alternative embodiments contemplate the provision of one or more syringes initially detached from a syringe coupler. In such embodiments, a user such as a healthcare professional assembles the devices by connecting one or more syringes to the syringe coupler just prior to conducting mixing operations.

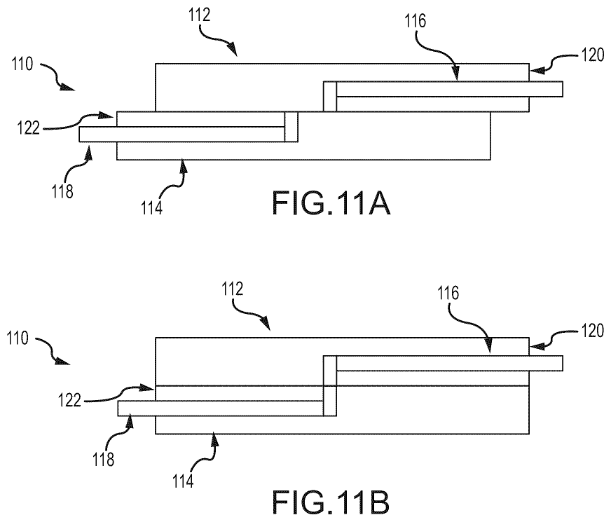

FIG. 11A is a cross-sectional elevation view of a mixing syringe component 110 according to one embodiment of the present disclosure. As shown, the component 110 comprises a valve element that is convertible between a first position (FIG. 11A) and a second position (FIG. 11B). As shown, the component 110 comprises first 112 and second 114 translatable components. The first and second translatable components 112, 114 are operable to be displaced relative to one another from a first position (FIG. 11A) wherein a conduit 116 of the first component 112 is unaligned with a conduit 118 of the second component 114. In the first position, the conduits 116, 118 are not connected and fluid flow between the components is substantially occluded. The first component 112 and second component 114 are displaceable to a second position (FIG. 11B) wherein the conduits 116, 118 have been brought into connection and/or aligned such that fluid flow between the components 112, 114 is enabled.

In certain embodiments, proximal ends 120, 122 of the components are operable to receive a syringe. An axial compression force on the syringe(s) (not shown in FIGS. 11A-11B) is operable to displace the components from the misaligned position of FIG. 11A to the aligned position of FIG. 11B in which fluid is allowed to pass between the components and related syringes. Although not shown in FIGS. 11A-11B, the proximal ends 120, 122 of the components are contemplated as comprising securing means for syringes. Securing means are contemplated as comprising, for example, threaded connection members, Luer lock components, and similar features to selectively secure a syringe to the components. In operation, a user may apply a compressive force to one or more syringes connected to the mixing syringe component 110 of FIGS. 11A-11B to displace the device from the position of FIG. 11A to the mixing position of FIG. 11B. The user may then proceed with mixing operations by sequentially applying a force to plunger rods of interconnected syringes as shown and described herein. The arrangement of the device of FIGS. 11A-11B therefore reduces process steps and reduces the need for a user to reposition their hands between activation of the valve element and a mixing operation.

It is further contemplated that the embodiments of FIGS. 11A-11B comprise stops or limiting members to prevent movement of the components beyond a desired position wherein mixing is enabled. The limiting members may further comprise resilient stops or connection members to secure the devices in a mixing position and prevent reverse translation.

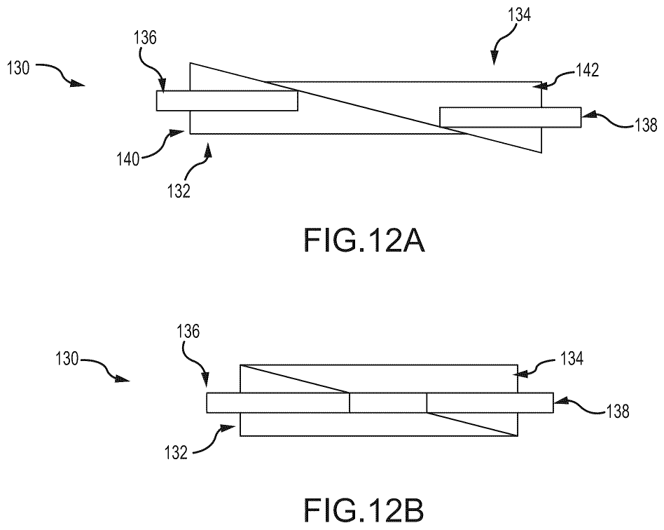

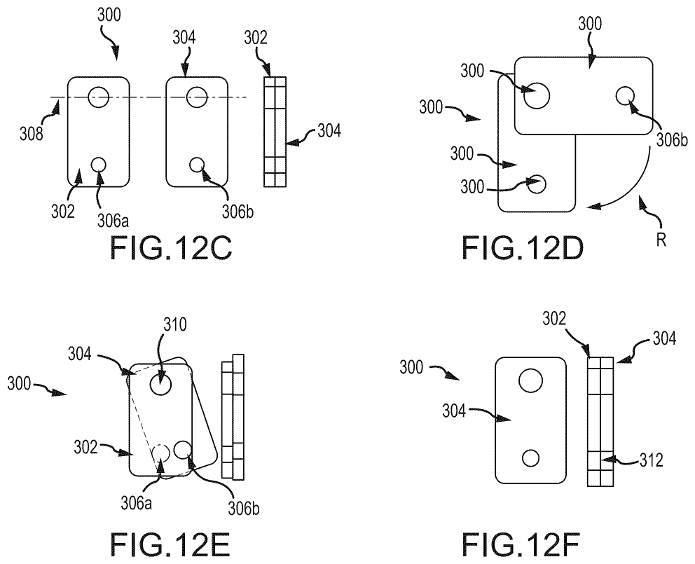

FIG. 12A is a cross-sectional elevation view of a mixing syringe component 130 according to one embodiment of the present disclosure. As shown, the component 130 comprises a valve element that is convertible between a first position (FIG. 12A) and a second position (FIG. 12B). As shown, the component 130 comprises first 132 and second 134 translatable components. The first and second translatable components 132, 134 are operable to be displaced relative to one another from a first position (FIG. 12A) wherein a conduit 136 of the first component 132 is unaligned with a conduit 138 of the second component 134. In the first position, the conduits 136, 138 are not connected and fluid flow between the components is substantially occluded. The first component 132 and second component 132 are displaceable to a second position (FIG. 12B) wherein the components 132, 134 provide a ramped or cammed surface, and wherein the conduits 132, 134 have been brought into connection and/or aligned such that fluid flow between the components 132, 134 is enabled.

In certain embodiments, proximal ends 140, 142 of the components are operable to receive a syringe. An axial compression force on the syringe(s) (not shown in FIGS. 12A-121B) is operable to displace the components from the misaligned position of FIG. 12A to the aligned position of FIG. 12B in which fluid is allowed to pass between the components and related syringes. Although not shown in FIGS. 12A-12B, the proximal ends 140, 142 of the components are contemplated as comprising securing means for syringes. Securing means are contemplated as comprising, for example, threaded connection members, Luer lock components, and similar features to selectively secure a syringe to the components. In operation, a user may apply a compressive force to one or more syringes connected to the mixing syringe component 130 of FIGS. 12A-12B to displace the device from the position of FIG. 12A to the mixing position of FIG. 12B. The user may then proceed with mixing operations by sequentially applying a force to plunger rods of interconnected syringes as shown and described herein. The arrangement of the device of FIGS. 12A-12B therefore reduces process steps and reduces the need for a user to reposition their hands between activation of the valve element and a mixing operation.

It is further contemplated that the embodiments of FIGS. 12A-12B comprise stops or limiting members to prevent movement of the components beyond a desired position wherein mixing is enabled. The limiting members may further comprise resilient stops or connection members to secure the devices in a mixing position and prevent reverse translation.

FIG. 12C illustrates a mixing syringe component 300 operable to selectively permit and restrict a fluid flow through the device and associated features. As shown, the component 300 comprises a valve element having a first portion 302 and a second portion 304. Each of the first portion and the second portion comprise an aperture or flow port 306a, 306b. When the flow ports 306a, 306b are offset or misaligned by a certain degree, flow through the component 300 is prevent. The first portion 302 and second portion 304 are rotatable at least relative to one another. Specifically, the portions 302, 304 are rotatable around an axis 308. A pin or axle member is contemplated as being provided to secure the portions and enable rotation. FIG. 12C depicts the first portion 302 and the second portion 304 in disassembled and assembled states.

FIG. 12D is an elevation view of the component 300 of FIG. 12C. The first portion 302 and the second portion 304 are rotationally offset to illustrate a motion and freedom of movement of the device 300. A rotational movement R is operable to place the first portion 302 and the second portion 304 in alignment and selectively enable fluid flow.

FIG. 12E shows the component 300 in a closed position wherein fluid flow is prevented or occluded. As shown, the second portion 304 is rotated relative to the first portion 302 such that the fluid flow ports 306a, 306b are offset and fluid cannot pass through the device 300. The position shown in FIG. 12E is contemplated as being an initial position wherein mixing and fluid flow are prevented.

FIG. 12F shows the component 300 in an aligned position wherein fluid flow is enabled. As shown, the first portion 302 and the second portion 304 have been rotated into alignment wherein the flow ports 306a, 306b are axially aligned and a flow path 312 is created through the component 300.

FIGS. 12C-12F contemplate and depict a device with portions that are rectilinear cubes. It will be recognized, however, that various alternative arrangements are contemplated. For example, the first and second portions shown in FIGS. 12C-12F are also contemplated as being provided as disc-shaped features and/or various other geometric shapes. Additionally, although not shown in FIGS. 12C-12F, one or more stops or détentes are contemplated as being provided to guide or limit an amount of relative rotation between the first portion and the second portion. For example, one or more stops are contemplated as being provided to secured or lock the device in the open position (FIG. 12F, for example). Additionally, a ramp or resistance member is contemplated wherein an initial resistance force is provided that must be overcome in order to open the device and prevent or reduce the risk of accidental activation.

Although not shown in FIGS. 12C-12F, various extensions or user-interface portions are contemplated. For example, the features shown in FIG. 12C-12F may be provided internal to or partially internal to a larger device, and an extension or trigger is provided to enable to control rotation of at least a portion of the component 300.

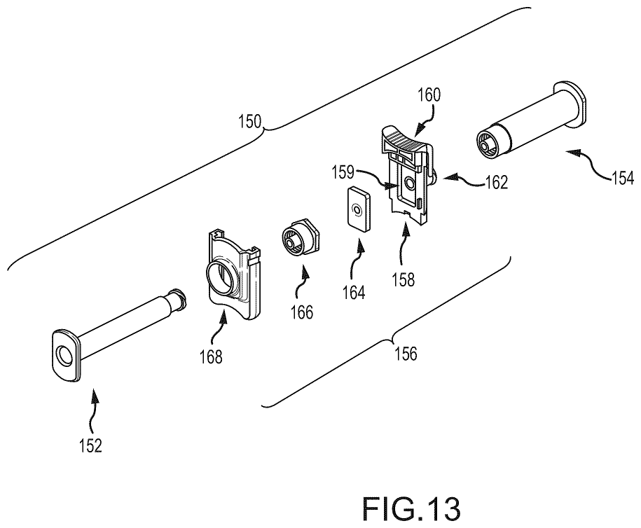

FIG. 13 is an exploded perspective view of a syringe-syringe mixing system 150 according to another embodiment of the present disclosure. As shown, the system 150 comprises a first syringe 152 and a second syringe 154. The first and second syringes are contemplated as initially comprising solid or liquid contents. For example, the first syringe 152 may house or comprise a polymer-solvent system such as, but not limited to, a biodegradable polymer dissolved in NMP and the second syringe 154 may comprise lyophilizate such as, but not limited to, lyophilized leuprolide acetate. Unwanted NMP migration (i.e. unintended migration prior to mixing) has been recognized as providing various complications including, for example, degrading or destroying shelf-life of contents. It is an object of various embodiments of the present disclosure to reduce or eliminate the risks of unwanted NMP migration while storing NMP and a drug lyophilizate in close proximity prior to mixing.

The contents of the first and second syringes 152, 154 may be mixed to formulate a solution or suspension for administration as shown and described herein. The embodiment of FIG. 13 comprises a syringe coupler 156. The syringe coupler 156 of the depicted embodiment is operable to receive and connect to the first and second syringes 152, 154, selectively prevent and enable fluid transfer between the two syringes, and selectively prevent removal of at least one syringe.

Each syringe 152, 154 comprises a barrel having an internal volume defined by a hollow body, proximal ends for receiving a plunger rod (not shown in FIG. 13), and distal ends with dispensing outlets wherein the distal ends are operable to connect to the syringe coupler 156. The syringe coupler 156 comprises a valve assembly with a sealing element 164 that nests within recessed area 159 of a displaceable member 158. In some embodiments, including that shown in FIG. 13, the sealing element comprises a fluid impermeable material with an aperture to selectively allow fluid to flow through the device 156. The sealing element 164 of FIG. 13 comprises a rectilinear member and is contemplated as having various shapes and sizes.

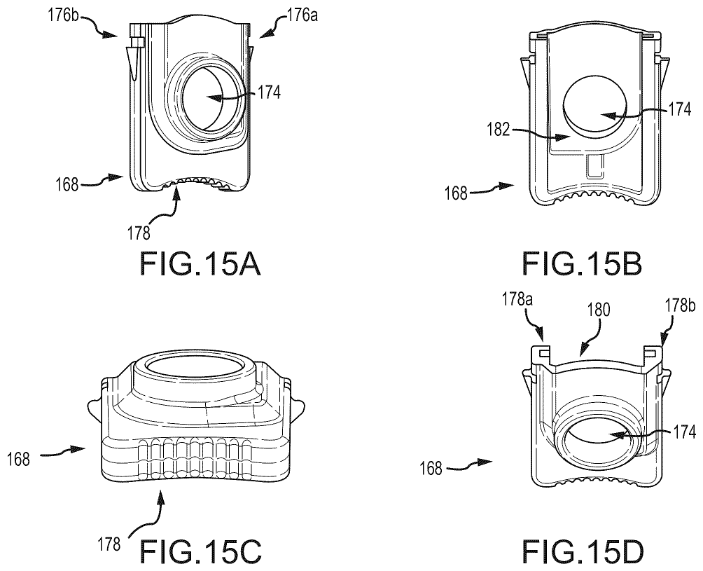

The displaceable member 158 comprises a user-interface 160 that is operable to be contacted by and receive a force from a user and a luer connection 162 for receiving the second syringe 154. The syringe coupler 156 further comprises a guide member 168 within which the displaceable member is provided. The guide member 168 comprises a user-interface 178 (FIG. 15C) that is operable to be contacted by and receive a force from a user. A rotatable member 166 is provided. The rotatable member 166 of the depicted embodiment comprises a rotatable Luer lock member with a proximal end with a male fitting operable to connect to the first syringe 152, and a distal end comprising a flange with a plurality of contact surfaces for limiting rotation of the rotatable member 166 prior to activation of the device.

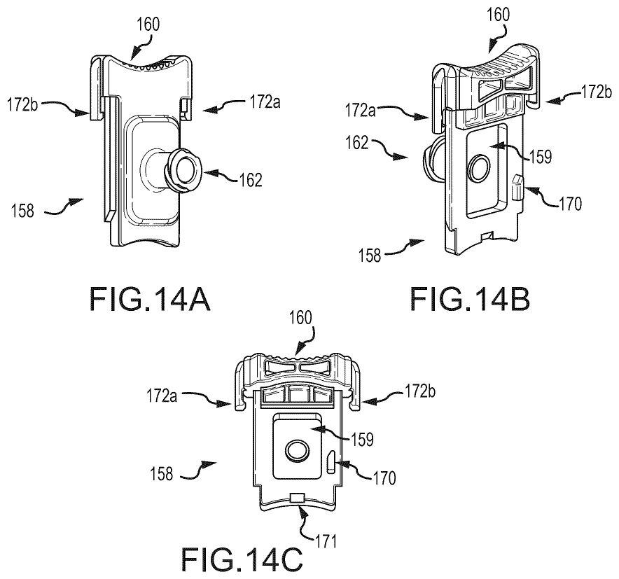

FIGS. 14A-14C are perspective views showing the displaceable member 158 in greater detail. As shown, the displaceable member 158 comprises a user-interface 160 operable to be acted upon by a user. In preferred embodiments, the displaceable member is displaceable in a downward direction (at least relative to FIG. 14A) and is preferably not operable to return to an initial or first position. A luer connection 162 is provided on one side of the member for receiving a syringe. A recess 159 is provided on an opposing side of the displaceable member relative to the luer connection 162. The recess 159 is operable to receive a sealing element, such as the sealing element 164 of FIG. 13. A channel is provided through the displaceable member 158, wherein the channel extends through the luer connection 162 and into the recess 159. Preferably, a sealing element comprises an aperture that is aligned with the channel of the displaceable member 158.

As shown in FIGS. 14B-14C, a projection 170 is provided on the displaceable member 158. The raised projection 170 is displaceable with the member 158 and is moveable relative to at least the rotatable member 166 of an assembled device. In a first position, the projection 170 is provided in contact with a contact surface of the flange of the rotatable member 166 to prevent rotation of the member 166. This contact and related locking of the rotatable member 166 enables a first syringe to be threaded onto (and threadably removed from) the rotatable member 166 prior to activation of the assembled device. Movement of the displaceable member 158 by user activation results in displacing the projection 170 such it is not in contact with rotatable locking member 166. With rotation of the rotatable Luer lock member enabled, the member 166 is free to spin within the syringe coupler. Without resistance, a syringe connected to the rotatable Luer lock member 166 is prevented from being threadably detached from the syringe coupler even if and when a rotation is applied in an attempt to remove the syringe. It is an object of the present disclosure to provide a syringe coupler 156 that retains at least one syringe such that a user is prevented from using the first syringe for administration and is thereby only given the option of administering the mixed contents with the second syringe.

As shown in FIGS. 14A-4C, the displaceable member 158 also comprises clips or resilient projections 172a, 172b. The resilient projections 172a, 172b are operable to flex outwardly and do not substantially impede a downward movement of the displaceable member 158. When provided in a second position, however, the resilient projections 172a, 172b are secured to the guide member 168 at least in part due to an inherent restoring force of the projections. The resilient projections 172a, 172b secure the displaceable member 158 in a second position within guide member 168 through engagement of said resilient projections 172a, 172b into recesses 176a, 176b located upon guide member 168 to prevent or inhibit the displaceable member from being returned to a first position.

FIGS. 15A-15D are perspective views of a guide member 168 according to one embodiment and contemplated for use and cooperation with the displaceable member 158 is FIGS. 14A-14C. As shown, the guide member 168 comprises a central aperture 174 to permit fluid flow and to receive a rotatable Luer lock member 166 of embodiments of the disclosure. The guide member 168 is provided to slidably receive at least a portion of a displaceable member 158. As shown, the guide member 168 comprises a receiving portion 180 with first and second slot members 178a, 178b to receive a displaceable member 158. The guide member comprises a user-interface 178 that is operable to be contacted by and receive a force from a user. In certain embodiments, and as shown in FIGS. 15A-15D, the user-interface 178 is contemplated as comprising a gripping or contact surface having ridges to reduce slipping and provide ergonomic benefits.

A surface of the guide member 168 comprises a recessed area 182 in which the projection 170 of the displaceable member 158 (FIG. 14B, for example) is allowed to translate. Specifically, when the coupler is activated and the displaceable member 158 is displaced downwardly relative to the guide member 168, the projection 170 downwardly in the recessed area 182 to a second position wherein the projection 170 is not in contact with the rotatable member 166 regardless of the rotational position of the rotatable member 166.

The second position further comprises a position wherein a fluid flow channel is created. Specifically, a sealing element 164 provided within the displaceable member 158 is moved from a first position characterized by a channel of the sealing element 164 being offset from and preventing flow between inlets and outlets of interconnected syringes and a second position characterized by the channel of the sealing element 164 being provided in axial alignment with the syringe outlets and inlets.

As shown in FIGS. 15A-15B, for example, the guide member 168 further comprises recesses 176a, 176b that are operable to receive resilient raised projections of a displaceable member and secure the syringe coupler in a second position. The recesses are operable to prevent or at least impede a user from returning the device to a first position after activation of the syringe coupler.

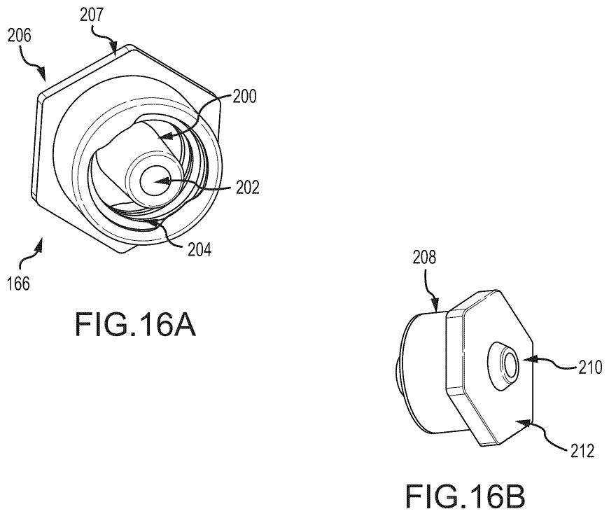

FIGS. 16A-16B are perspective views of a rotatable member 166 according to one embodiment of the present disclosure. As shown, the rotatable member 166 comprises a first end with a male Luer lock 200 that provides a means of attachment to a first syringe as well as a fluid flow path through a central aperture 202 of the member 166. The male Luer lock 200 is at least partially provided within a threaded female member 204 that is operable to threadingly engage a first syringe. A bearing surface 208 is provided on an exterior of the member 166. The bearing surface 208 is operable to be provided in the central aperture 174 of the guide member 168 and contact the guide member. The bearing surface 208 of the rotatable member 166 comprises a surface upon which the member 166 can rotate (when unlocked) and contact the central aperture 174 of the guide member 168. The rotatable member 166 further comprises a flange 206 with a plurality of contact surfaces 207 to selectively prevent rotation of the rotatable member 166. Specifically, when a projection of the present disclosure (170 of FIG. 14B, for example) is provided in a first position, the projection 170 is provided in contact with at least one contact surface 207 of the flange 206 and rotation of the member 166 is prevented (at least with respect to the guide member 168). The secured nature or state of the member 166 in the first position allows a user to thread a first syringe within the threaded female member 204. When the displaceable member is displaced as shown and described herein, the projection 170 is moved away from the flange 206 of the member 166 such that rotation is unopposed and the member 166 is allowed to rotate relative to the guide member 168 and the displaceable member. This freedom of rotation prevents or at least inhibits the un-threading and removal of the first syringe as a rotation force applied to the syringe will cause a rotation of the rotatable member 166. With no significant oppositional force on the rotatable Luer lock member 166 or threaded female member 204, un-threading will not occur and the first syringe is effectively prevented from being removed from the syringe coupler. The flange 206 of the embodiment of FIGS. 16A-16B is depicted as a hexagonal flange. It will be recognized, however, that various alternative shapes and arrangements are contemplated that comprise at least one contact surface for selectively preventing rotation of the member 166.

FIG. 16B shows the distal end of the channel 202 of the rotatable member 166. As shown, the distal end 210 comprises a ramped or frustoconical shape. The angled surface(s) of the distal end 210 allow for ease of assembly of the device. Specifically, the distal end 210 is operable to communicate with a slot or ramp 171 of the displaceable member 158 (FIG. 14C, for example). The angled nature of these corresponding features allows the guide member 168 comprising the rotatable member 166 to slide relative to and be assembled with the displaceable member 158. As previously discussed, the displaceable member is contemplated as comprising a sealing member 164. In addition to facilitating ease of assembly, the communication of the distal end 210 and the ramp 171 of the guide member provides for a temporary separation of the guide member 168 and the displaceable member and avoid unwanted contact, damage, or snagging of the seal 164 during assembly. Upon full insertion of the displaceable member 158 within the guide member 168, the distal end 210 is provided proximal to the sealing member 164.

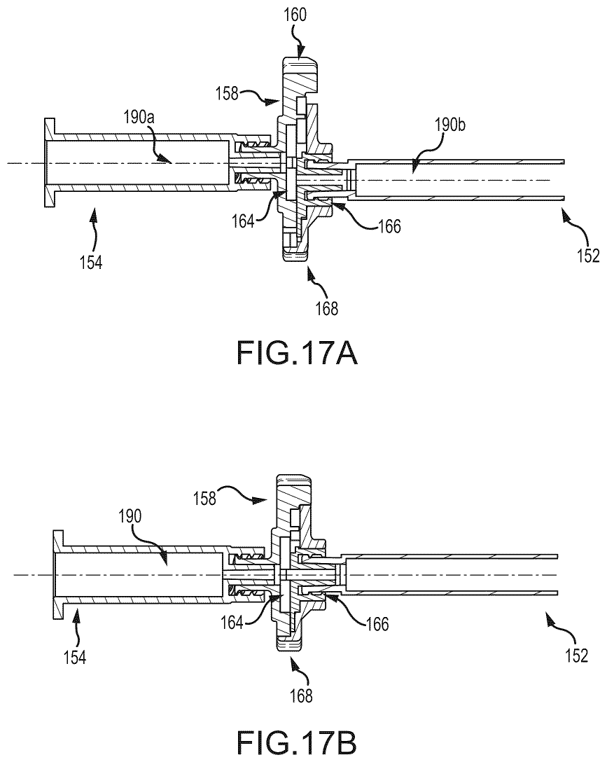

FIGS. 17A-17B are cross-sectional elevation views of a system according to an embodiment of the present disclosure. As shown and previously described, the system comprises a first syringe 152 and a second syringe 154. The syringes 152, 154 are connected to a syringe coupler comprising a displaceable member 158 with a user-interface 160, a sealing element 164, a guide member 168, and a rotatable member 166 provided at least partially within the guide member 168. The system is shown as being provided in a first position in FIG. 17A. The first position comprises a position wherein the displaceable member and associated sealing element 164 are provided offset from a central axis and passageway of the rotatable member 166. Specifically, a fluid flow path 190a of the second syringe 154, the male extension of the displaceable member 158, and the sealing member 164 is offset from and not in communication with a fluid flow path 190b of the first syringe 152 and the rotatable member 166. Fluid and gaseous vapor flow between syringes is thus prevented.

FIG. 17B depicts the system in a second position wherein the displaceable member 158 has been displaced by application of force upon the user-interface 160 (for example). As shown, fluid pathway 190a of FIG. 7A and related components have been displaced such that a continuous fluid pathway 190 is provided and fluid flow between the first syringe 152 and second syringe 154 is enabled. Mixing of contents is thus enabled, wherein plunger rods (not shown in FIGS. 17A-17B) associated with the first and second syringes are operable to force contents between the syringes.

In various embodiments, a first syringe 152 is initially provided with a liquid formulation component (i.e. liquid or flowable material) such as a polymer-solvent system and a second syringe is provided with an API, which may, in some non-limiting instances, be present as a lyophilized powder. In such embodiments, the contents are stored separately with each respective syringe, which are interconnected to the syringe coupler with the displaceable member provided in the first position (FIG. 17A). Additional embodiments further contemplate the provision of a liquid in each of the two syringes associated with the system(s). To administer a therapeutic agent, the displaceable member is depressed or otherwise activated, creating the fluid-flow pathway 190 of FIG. 17B. Repetitive mixing may then be performed by forcing the contents (e.g. polymer-solvent) of the first syringe 152 into the second syringe that comprises additional contents (e.g. API), forcing the contents back to the first syringe, and repeating the process until desired mixing is achieved. As discussed, the second position of FIG. 17B is characterized by the presence of a fluid flow path between the two syringes, as well as by the disengagement of the displaceable member 158 and the rotatable member 166. Specifically, the second position (FIG. 17B) comprises a position in which the rotatable member 166 is free to rotate within the syringe coupler and the first syringe 152 is prevented from unthreading or detachment. Accordingly, the second syringe preferably comprising the mixed or prepared agent is detachable for use as an injection syringe while the first syringe is inoperable for such purposes. Systems, devices and methods of the present disclosure are not limited to any particular therapeutic agent(s), solution(s), suspension(s), gas(es), or a combination thereof. Various embodiments comprise features and sealing elements for preventing materials in at least one syringe from escaping or migrating to another syringe. In some embodiments, for example, it is contemplated that that one or more non-lyophilized materials are provided in syringes of the present disclosure. In some embodiments, a gas (e.g. Nitrogen or Argon gas) is provided in a syringe for mixing with contents of a second syringe. Gas may be desirable, for example, to be provided with an active pharmaceutical ingredient to preserve that ingredient during storage. Sealing elements of the present disclosure are operable to and suitable for maintaining gas in a syringe and preventing unwanted migration of that gas. Sealing elements are also suitable and operable for preventing escape or flow of liquids and solids. In some embodiments, mixing syringe systems of the present disclosure comprise gas-impermeable materials to prevent gas permeation and migration.

As disclosed herein, the syringe mixing system of the invention may comprise methods and systems for mixing components of a pharmaceutical composition or formulation comprising an API useful in the treatment in a disease or disorder in a patient. In some embodiments, the syringe mixing system comprises a first syringe containing a first gas, liquid, or solid composition and a second syringe containing a second gas, liquid, or solid composition. Upon activation of the syringe connector from a first, closed position to a second, open position, the first gas, liquid, or solid composition of the first syringe may be intermixed with the second gas, liquid, or solid composition of the second syringe (or vice versa) until a desired intermixed composition is formed. In some instances, the first or second syringe (but not both) may contain a gas component which may be an inert or volatile gas or gas vapor. In some instances, the first and second syringe may contain an aqueous based or organic based liquid which forms a solution, suspension, or both. In some further instances of the disclosed invention, the first syringe may comprise liquid formulation component or a solvent system which may, in some non-limiting examples, contain a biodegradable polymer dissolved or suspended within an aqueous, organic, or intermixed aqueous-organic solvent system, which may further contain additional co-solvents. In some instances, the first or second syringe (but not both) may contain a solid which may be an API useful in the treatment of a disease or disorder or amelioration of a symptom thereof. In some further instances, the solid may be a lyophilized powder, semi-solid particulate(s), or solid particulate(s) of varying sizes, shapes, and characteristics (e.g. specific surface area for example). Yet, further still, in some other non-limiting instances, the first or second syringe of the syringe device system may comprise a lyophilized powder, semi-solid particulate(s), or solid particulate(s) of varying sizes, shapes, and characteristics (e.g. specific surface area for example) which may be prepared and/or stored within the first or second syringe within the presence of a gas of choice, i.e. both lyophilized powder and gas are contained within the first or second syringe prior to mixing said components with the components stored within the opposing syringe, which may be, but is not necessarily limited to, a liquid of interest. In various embodiments, an API that is at least partially dissolved or suspended in a liquid which may contain a solvent, excipient, polymer, and/or other material is provided in one syringe. The other syringe is contemplated as comprising the same or similar contents without an API, or an API of 1) the same or different amount, 2) the same or different salt forms, 3) the same or different polymorphic form of the API, 4) the same or different prodrug forms, or 5) different compositions entirely (e.g. two or more distinctly unique APIs). It will be recognized, however, that the present disclosure is not limited to any particular arrangement or provision of materials within syringes. For example, certain embodiments of the present disclosure contemplate methods and systems of storing and then mixing two initially separated liquid components. In some further embodiments, the present disclosure contemplates methods and systems of storing and then mixing two initially separated liquid components, wherein both components further comprise one or more solvents and wherein one or both components further comprise one or more APIs, which may be partially or fully dissolved or suspended in said solvent(s). Additional embodiments of the present disclosure contemplate that at least one of two mixing syringe comprises a combination of a liquid and a solid prior to mixing.

As disclosed herein, the syringe mixing system of the invention may comprise methods and systems for mixing components of a pharmaceutical composition or formulation comprising an API useful in the treatment of a disease or condition in a patient. Such a syringe mixing system may be referred to as a “prefilled syringe mixing system” or a prefilled “syringe-to-syringe mixing system”, wherein the syringes of the syringe mixing system are prefilled with components of a pharmaceutical composition or formulation that are then mixed together using the syringe mixing system as described herein, such that the mixed pharmaceutical composition or formulation can then be administered to a patient in need of such pharmaceutical composition or formulation. In some embodiments of the invention, the syringe mixing device (prefilled syringe mixing system) may comprise a pharmaceutical formulation comprising: (a) an API, which is contained within one syringe, and (b) a biodegradable polymer-solvent system contained within the other syringe, which may be intermixed upon activation of the syringe connector by a user such as to prepare a medication or medicament useful in the treatment of a disease or condition by administration of the mixed formulation into a patient in need thereof. The syringe mixing system can be used to store and then mix for administration any pharmaceutical composition or formulation that would benefit from the advantages of the inventive syringe mixing system, and the disease or condition to be treated will naturally depend on the drug or therapeutic agent included in the pharmaceutical composition or formulation.

In some embodiments, the API is a Gonadotrophin Releasing Hormone (GnRH) agonist or antagonist or a pharmaceutically acceptable salt thereof. Diseases or conditions that may be treated with a GnRH agonist or antagonist or a pharmaceutically acceptable salt thereof may include, but are not limited to, certain types of cancers, central precocious puberty (CPP), endometriosis, or uterine fibroids. In some instances, a cancer that may be treated with a GnRH agonist or antagonist or a pharmaceutically acceptable salt thereof may include but is not limited to prostate cancer (including but not limited to advanced prostate cancer) or breast cancer.

Leuprolide, as known as leuprorelin, is a synthetic peptide analog that acts as a “super agonist” upon pituitary GnRH receptors. GnRH agonists, such as leuprolide or a pharmaceutically acceptable salt thereof (such as leuprolide acetate), may be used in the treatment of prostate cancer (including advanced prostate cancer) in adult males, HR-positive breast cancer (including, but not limited, to HR-positive, human epidermal growth factor receptor 2 (HER2)-negative breast cancer) and CPP. Administration of GnRH agonists (or GnRH) leads to downregulation of GnRH receptor activity, which in turn downregulates GnRH-dependent secretion of gonadotropins, including but not limited to, luteinizing hormone (LH) and follicle-stimulating hormone (FSH). Downregulation of LH and FSH leads to subsequent down-regulation of secondary sex-hormones, including but not limited to, testosterone and estradiol. Testosterone is a key metabolite in driving prostate cancer development and progression in adult males. As such, the reduction of serum testosterone levels is a useful clinical approach for slowing or inhibiting the growth of prostate cancer. Likewise, clinical approaches that modulate hormone activity and/or synthesis, particularly that of estrogens (e.g. estradiol), are useful for slowing or inhibiting the growth of hormone receptor-positive (HR-positive) breast cancer. Controlled release formulations for the extended release of leuprolide useful in the treatment of the prostate cancer in adult males, breast cancer, and CPP in pediatric patients 2 years old or older have been developed. Controlled release formulations using flowable biodegradable polymer-based compositions for sustained, extended release of leuprolide or pharmaceutically acceptable salts thereof have been described, by way of example, in U.S. Pat. Nos. 6,565,874 and 8,470,359, WO 2020/2404170, and WO 2020/217170, each of which are incorporated herein by reference in their entireties.

As disclosed herein, the syringe device or mixing system may be used to administer an API, intramuscularly (IM) or more preferably subcutaneously (SQ), to a patient in need thereof. In some embodiments, the API is a GnRH agonist or antagonist or a pharmaceutically acceptable salt thereof and the patient may suffer from prostate cancer, hormone receptor-positive breast cancer, or CPP. In some embodiments, the method of administering the GnRH agonist or antagonist or the pharmaceutically acceptable salt thereof comprises mixing a unit dose of the GnRH agonist or antagonist or the pharmaceutically acceptable salt thereof with a liquid formulation component to form a reconstituted pharmaceutical composition using the syringe-to-syringe mixing system; and administering the reconstituted pharmaceutical composition to the patient via subcutaneous injection. In some embodiments, the syringe-to-syringe mixing system comprises a first syringe barrel comprising the liquid formulation component, a second syringe barrel comprising the GnRH agonist or antagonist or the pharmaceutically acceptable salt thereof, and a syringe coupler comprising a displaceable member, wherein the displaceable member comprises a seal with a flow port that is offset from an outlet of at least one of the first syringe barrel and the second syringe barrel when the displaceable member is provided in a first position, and wherein the flow port is aligned with the outlet of the first syringe barrel and the second syringe barrel when the displaceable member is provided in a second position, and wherein the displaceable member is displaceable in a direction that is substantially perpendicular to a longitudinal axis of at least one of the first syringe barrel and the second syringe barrel. The mixing comprises applying a force to a user-interface to move the displaceable seal from the first position to the second position and applying force to a plunger positioned in the first syringe barrel and a plunger positioned in the second syringe barrel in an alternating manner to mix the contents of the first syringe barrel and the second syringe barrel. In some instances, the GnRH agonist or antagonist or the pharmaceutically acceptable salt thereof is leuprolide or a pharmaceutically acceptable salt thereof, such as leuprolide acetate. In some embodiments, the GnRH agonist or antagonist is provided in the form of a liquid to be mixed.

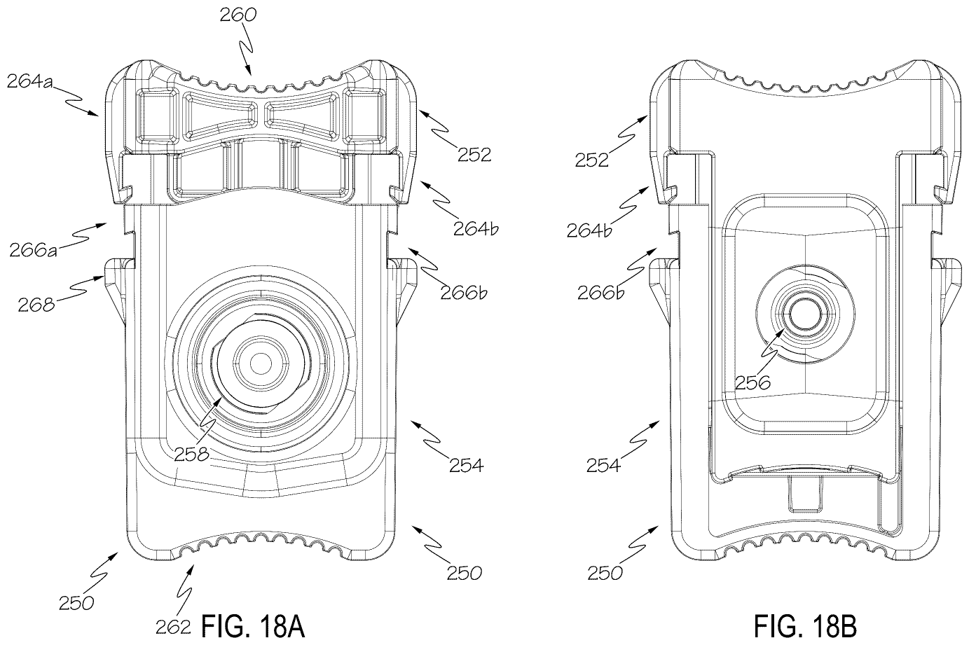

FIGS. 18A-18B are elevation views of opposing sides of a component 250 of a mixing syringe assembly according to an embodiment of the present disclosure. As shown, a displaceable member 252 and a guide member 254 are provided in a first position. The first position is suitable for shipping and storage wherein fluid and gas vapor flow between interconnected syringes is fully or at least partially occluded and wherein fluid is contained within each respective syringe prior to activation and mixing. The displaceable member 252 comprises an interconnected sealing element 256. The displaceable member provides a rigid housing member for the sealing element 256 in addition to the various additional features and benefits as shown and described herein. The sealing element 256 comprises an aperture, but the aperture is offset from the fluid flow path of the guide member 254 and rotatable luer lock member 258 such that fluid flow through the device is occluded. The displaceable member 252 and the guide member 254 comprise user-interface portions 260, 262, respectively. Force may be applied to one or more of the user-interfaces 260, 262 to convert the device from the first position to a second position wherein the displaceable member 252 is displaced relative to the guide member and a fluid flow path is created.

The displaceable member 252 comprises first and second projections 264a, 264b that are operable to be outwardly displaced upon downward movement of the displaceable member. The first and second projections 264a, 264b are secured within the recesses 266a, 266b of the guide member 254 and move inwardly based on their inherent material properties and elasticity. The placement of the first and second projections 264a, 264b within or partially within the recesses 266a, 266b of the guide member 254 prevent or inhibit a return movement of the displaceable member 260 back to the first position. Various portions of the device 250 of FIGS. 18A-18B are similar to those and described with respect to FIG. 8. Various features, however, are provided with rounded or smooth corners and transitions. For example, and without limitation, various portions 268 are provided with rounded corners and edges so as to reduce a likelihood that the device 250 will puncture or tear associated packaging.

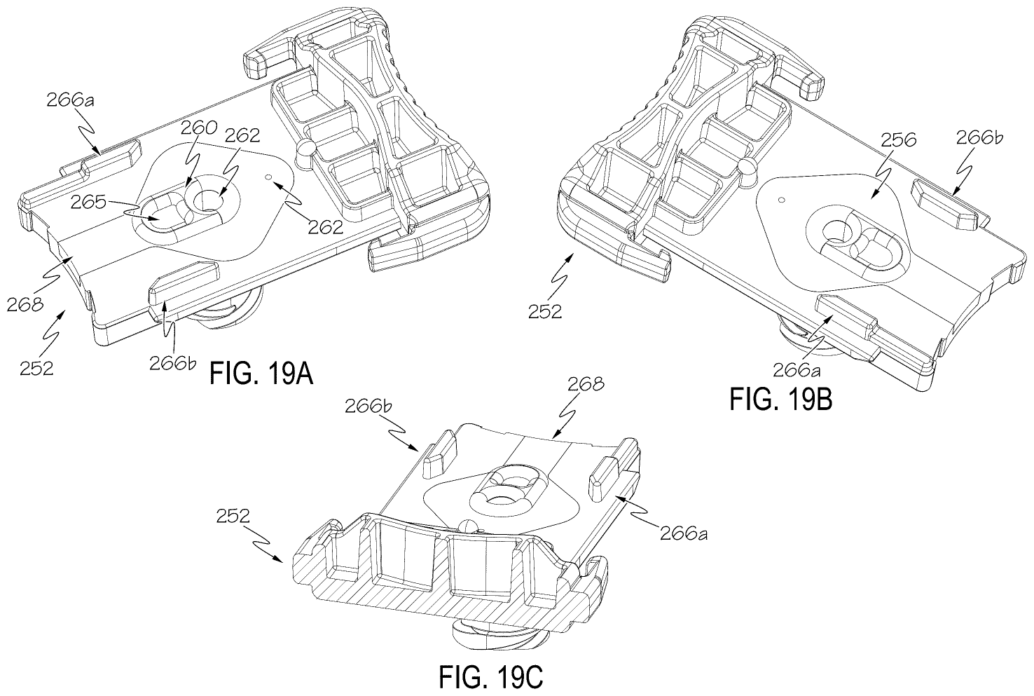

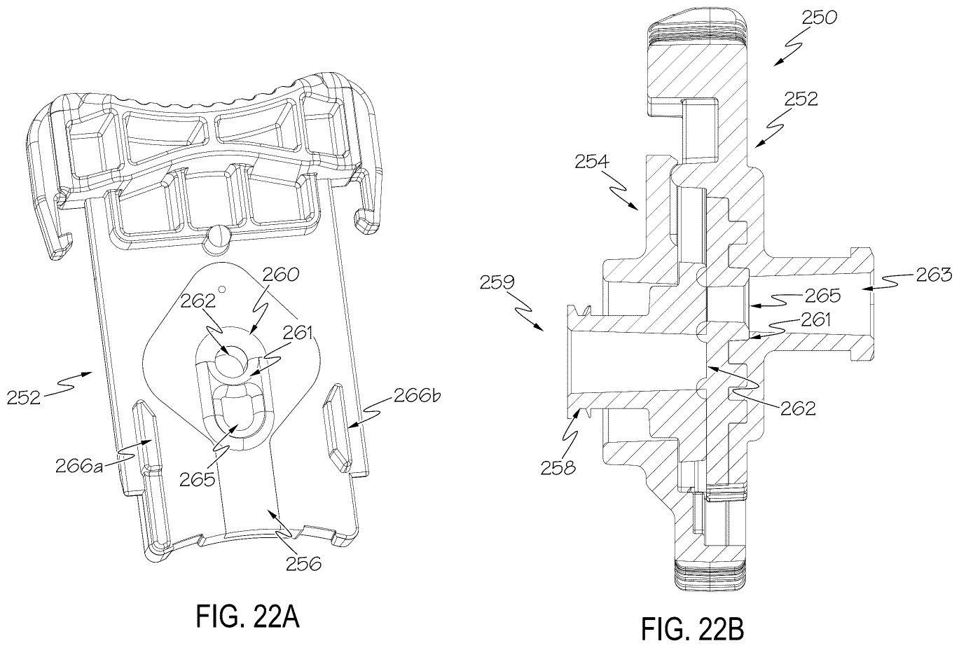

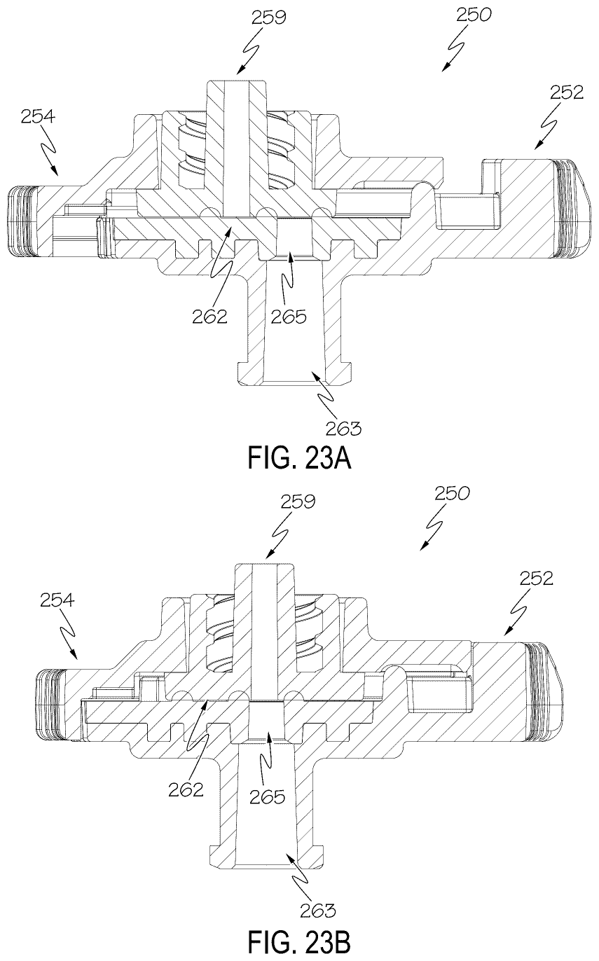

FIGS. 19A-19C provide various perspective views of a displaceable member 252 according to an embodiment of the present disclosure. The displaceable member 252 is shown in isolation in FIGS. 19A-19C for illustrative purposes and is contemplated as being provided with and cooperating with additional system components as shown and described herein. The displaceable member 252 comprises a sealing element 256 that is operable to substantially prevent the flow or transmission of at least one of a solid and a liquid. It is contemplated, for example, that syringe devices connected to the system comprise at least one of a solid and a liquid to be mixed and wherein such components or ingredients are to be reliably sealed and segregated prior to mixing. To provide such a reliable seal, a sealing element 256 is contemplated that comprises a main body portion and an embossed and/or upstanding portion 260 (referred to in subsequent description as the “upstanding portion 260”). As shown, the upstanding portion 260 is raised above the main body portion and generally forms two discrete areas 262, 265 surrounded by a raised seal or gasket. A first area 262 is defined that comprises an aperture or flow port. This area 262 is contemplated as being offset from a flow port provided in additional components (not shown in FIGS. 19A-19C but see rotatable luer lock of FIG. 22B, for example) at least in a first position. A second area comprises a sealed area that is operable to communicate with and seal the flow port at least when the device is provided in a first position (i.e. a storage or non-use position). The upstanding portion 260 also encompasses the second, smaller area 265, which is operable to surround a flow path of an adjacent component and fully occlude and segregate fluid from flowing to or through the offset first area 262. The upstanding portion 260 is sized and operable to provide a simultaneous seal to the first and second syringes in at least a stored or pre-mixing position. In various embodiments, the upstanding portion comprises a double O-ring or two part seal wherein annular or semi-annular projections are provided adjacent to one another and are operable to provide a seal to respective associated syringes.

Without limitation to any particular material(s), it is contemplated that the displaceable member can comprise or consist of a plastic such as polypropylene and the sealing element 256 can comprise or consist of a resilient member such as thermoplastic elastomer (e.g. Santoprene, silicone, or similar stable rubber-based materials and pharmaceutically acceptable thermoplastics). The plastic provides appropriate material properties such as strength, weight, and durability while the elastomer provides appropriate sealing performance.

As further shown in FIGS. 19A-19C, the sealing element comprises an extended sealing surface 268 that extends to or proximal to a lowermost portion(s) of the displaceable member 252. The extended sealing surface 268 provides for enhanced sealing and reduces an amount of plastic-on-plastic surface contact area which is prone to wear. The sealing surface 268 also facilitates and enables efficient production and assembly of the device as it provides clear and logical placement and registration of components. A sliding surface is thus provided that comprises a rubber-plastic interface which is less likely to form plastic debris that could damage the device or contaminate a drug associated with the system. For example, the avoidance of certain plastic-plastic interfaces reduces the risk and amount of microplastic formation and related contamination of pharmaceutical agents with plastic(s) or microplastic(s) due to frictional engagement and related abrasion and wear of parts. The arrangement of the seal is further operable to prevent or reduce deformation of the seal and seal components during assembly.

The displaceable member 252 as shown further comprises first and second detents 266a, 266b that extend from the displaceable member 252. The first and second detents 266a, 266b are operable to contact a rotatable member and prevent rotation thereof at least when the displaceable member 252 is provided in a first position. The plurality of detents provided in the depicted embodiment distribute a load that is provided when a syringe is threaded onto the device, for example, at least relative to a system comprising a single detent or projection. Furthermore, first and second detents 266a and 266b act to hold the rotatable member in proper alignment during assembly of the connector by preventing any movement of the rotatable member.

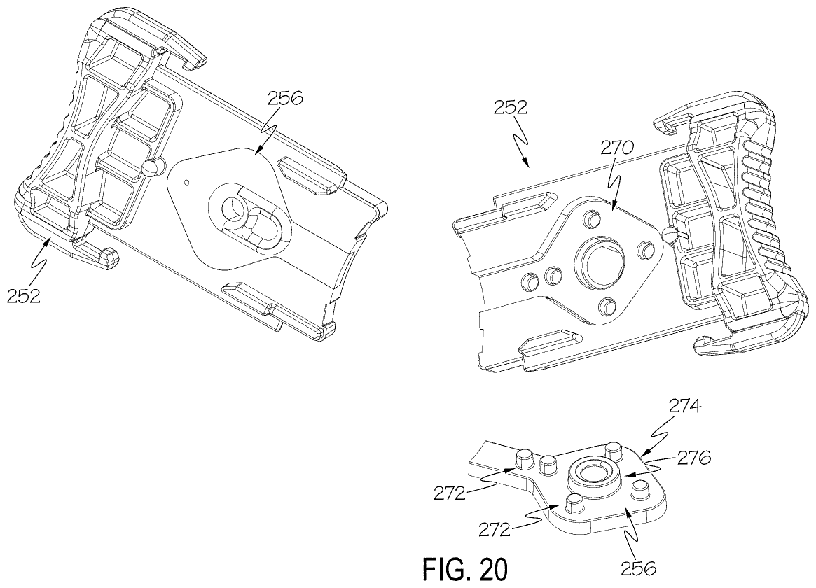

FIG. 20 is a perspective of a displaceable member 252 and a sealing element 256 in a first, assembled state and a second, disassembled state. As shown, the displaceable member 252 comprises a recessed receiving area 270 to receive at least a portion of the sealing element 256. The receiving area 270 generally comprises a complementary shape relative to the sealing element 256 and the sealing element is preferably molded into the receiving area 270. Projections 272 are provided on the sealing element 256 in some embodiments to register with corresponding recesses in the receiving area 270, assist with proper alignment and assembly, and prevent unwanted sliding and/or rotation of the sealing element 256 during assembly and/or activation. It is further contemplated in additional embodiments that the sealing element 256 and the displaceable member 252 are bonded to one another and do not require separate assembly outside of the molding process. As also shown in FIG. 20, a syringe-facing side 274 of the sealing element 256 comprises an annular projection 276 that is operable to provide sealing with a port or aperture in the displaceable member 252.

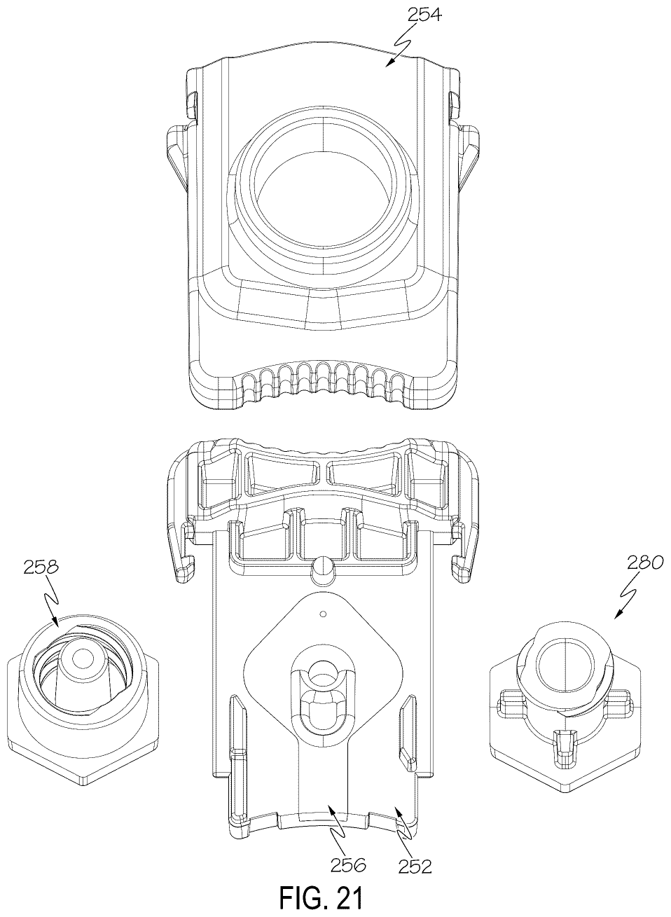

FIG. 21 is an exploded view of device according to an embodiment of the present disclosure. As shown, the device comprises a displaceable member 252 that is operable to communicate with and be displaceable with a guide member 254. First and second luer lock members 258, 280 are provided in different embodiments. As shown, one or two or more of the luer lock members 258, 280 are contemplated as being provided in order to connect to a female luer syringe or a male luer syringe, for example. The rotatable luer lock member 258 and/or the threaded luer lock member 280 receive a syringe to enable mixing.

FIG. 22A is a perspective view of the displaceable member 252 according to an embodiment of the present disclosure. FIG. 22B is a cross-sectional elevation view of the displaceable member 252 in an assembled but non-activated state with the guide member 254. FIG. 22B illustrates the assembled device 250 in a first position wherein fluid flow is not enabled and the device is provided in a sealed state. The sealing element 256 is positioned such that the upstanding portion 260 of the sealing element fully occludes or prevents fluid from flowing between a first syringe and a second syringe. More specifically, the upstanding portion 260 comprises a first area 262 and a second area 265. The first and second areas are divided by a septum 261. The first area 262 comprises a flow port that allows for fluid flow when the device is provided in a second position. The second area 265 comprises a sealed area that prevents fluid flow between connected syringes when the device is provided in a first position. The non-activated sealed state arrangement is shown in FIG. 22B wherein the second area 265 of the sealing element 256 is aligned with the flow path 259 of the rotatable luer lock 258. The upstanding portion 260 including the septum 261 provides a seal that separates the flow path 263 of the displaceable member 252 from the flow path 259 of the rotatable luer lock 258. Downward displacement of the displaceable member 252 from the position shown in FIG. 22B will bring the first area and the flow path 263 into alignment with the flow path 259 of the rotatable luer lock 258 such that liquid-liquid or liquid-solid mixing can be achieved.

FIGS. 23A and 23B depict the device 250 in the assembled, non-activated sealed position and the activated mixing position, respectively. Embodiments and devices of the present disclosure contemplate fluidic sealing in at least two respects. First, as illustrated in FIGS. 23A-23B, devices of the present disclosure contemplate a seal characterized by the occlusion and prevention of fluid flow between the guide member 254 and the displaceable member 252 (FIG. 23A) and therefore, occlusion and prevention of fluid flow between first and second syringes that are connected to the device.

A second mode or type of sealing is characterized by the prevention of leakage or fluid flow around the upstanding portion 260. This sealing is provided and applicable regardless of whether or not the device is in the first position or the second position. For example, the upstanding portion 260 provides an annular or semi-annular seal in the first position (FIGS. 22B, 23A) and in the second position or activated position (FIG. 23B, for example). Fluid flow along the path 262 of FIG. 23B is enabled and the device is thus not sealed in one respect. Sealing is provided in the second mode of sealing and wherein fluid is not allowed to bypass the upstanding portion 260 even in the activated position. Fluid contents are therefore constrained between syringes and the internal fluid flow pathway of the device.

The sealed position of FIG. 23A is substantially the same as that shown and described relative to FIG. 22B. FIG. 23B shows the device 250 post activation and wherein the first area 262 of the sealing element has been brought into alignment to create a fluid flow path between the port 259 of the rotatable luer lock and the port 263 of the displaceable member 252 and such that any connected syringe barrels of the device are provided in fluid communication with each other such that mixing is enabled.

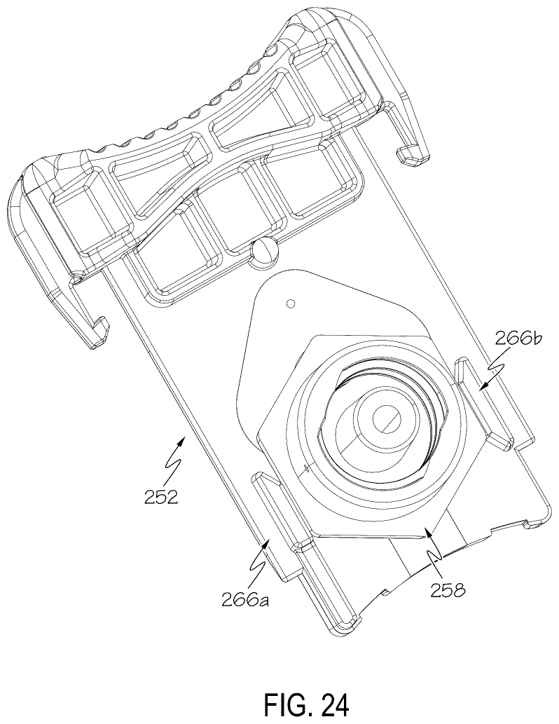

FIG. 24 is a perspective view of a displaceable member 252 according to an embodiment of the present disclosure. For illustrative purposes, a rotatable luer lock 258 (or 280) is shown. The rotatable luer lock is contemplated as comprising a receiving portion for a syringe and, in some optional embodiments, comprises a polygonal portion with a plurality of flat surfaces. The flat surfaces of the polygonal portion are operable to contact detents 266a, 266b to prevent rotation of the luer lock when the luer lock member 258 is brought into contact with the detents 266a, 266b. The operation of the embodiment of FIG. 24 is similar to that shown and described in FIGS. 16A-16B. More specifically, opposing flat surfaces of the luer lock member 258 are in contact with the detents 266a, 266b when the displaceable member 252 is provided in the sealed position. The opposition of rotation provided by the substantially rigid detents 266a, 266b allows for threading a syringe onto the device during assembly. In the mixing position (FIG. 23B, for example), the rotatable member 258 is moved out of contact with the detents 266a, 266b resulting in a freely rotatable luer lock member 258. This lack of opposition to rotational forces substantially prevents a user from unthreading a connected syringe as rotational forces applied to the syringe will simply cause rotation of the syringe around its longitudinal axis. In some embodiments, devices of the present disclosure are assembled or partially assembled by mechanized or robotic features that apply a predetermined amount of torque and may present a risk of over-torquing certain features. Devices and features of the present disclosure contemplate and provide factors of safety and load distribution to prevent or minimize damage during assembly, for example.

Although various embodiments, including that shown in FIG. 24, contemplate the provision of detents capable of contacting and providing force to the rotatable member, it should be recognized that alternative arrangements are contemplated for opposing and allowing rotation. For example, various contact surfaces, mating of teeth, and/or rack-and-pinion arrangements are contemplated to initially oppose rotation and allow rotation after activation of the device and/or repositioning of certain components.

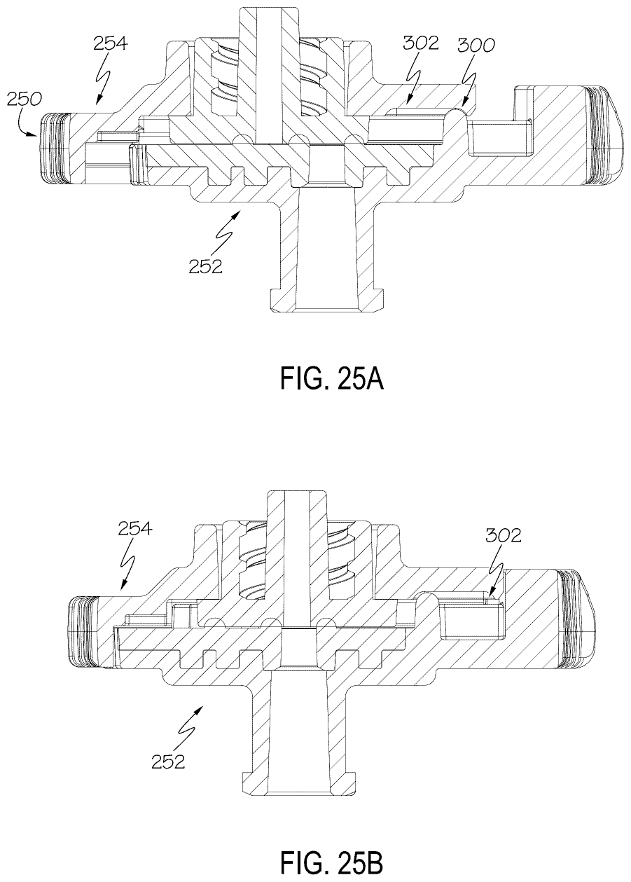

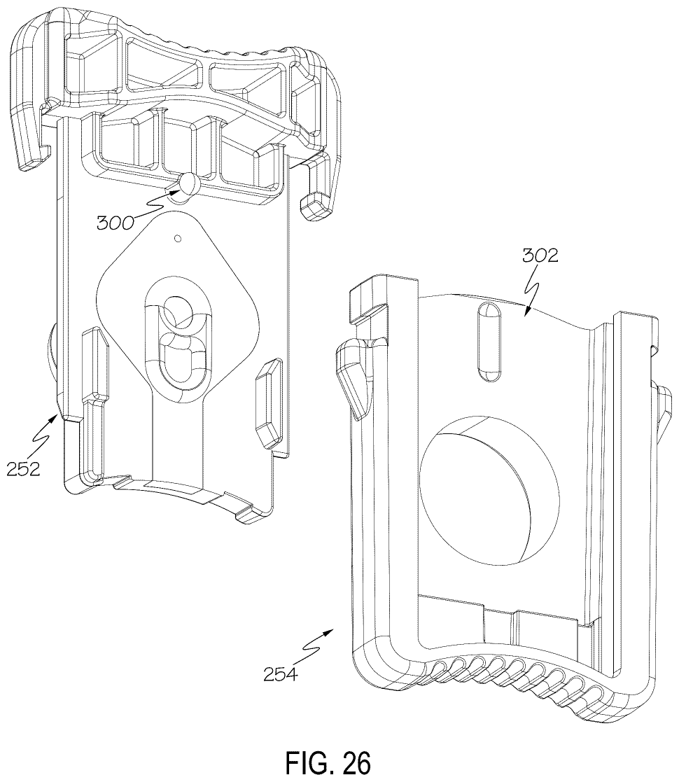

FIGS. 25A and 25B are cross-sectional elevation views of the device 250 in the sealed position and the mixing position, respectively. FIG. 26 is an exploded perspective view of components of the device. As shown, the device 250 comprises a projection 300 formed on an interior portion of the displaceable member 252. The projection 300 is provided at least partially within a slot 302 in the guide member 254. The communication between the projection 300 and the slot 302 serves to guide translation of the components relative to one another and prevent unwanted rotation of the components relative to one another. Distal ends of the slot 302 further comprise limits or stops for the translation to impede, for example, excessive translation or movement of components, and to prevent disassembly.

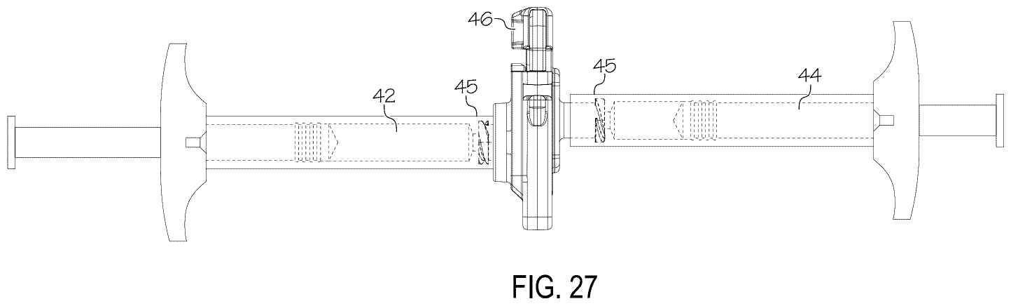

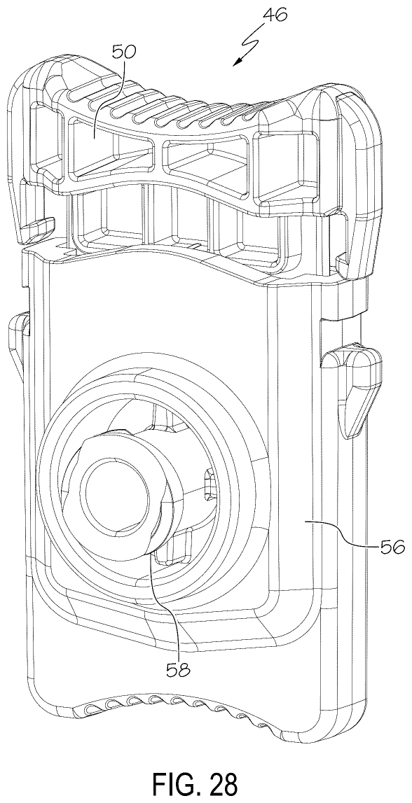

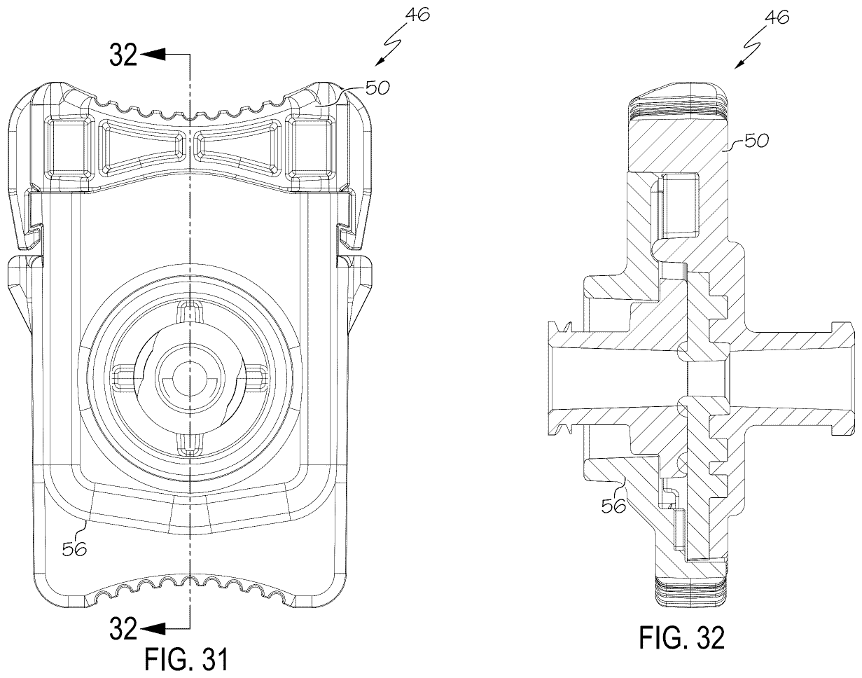

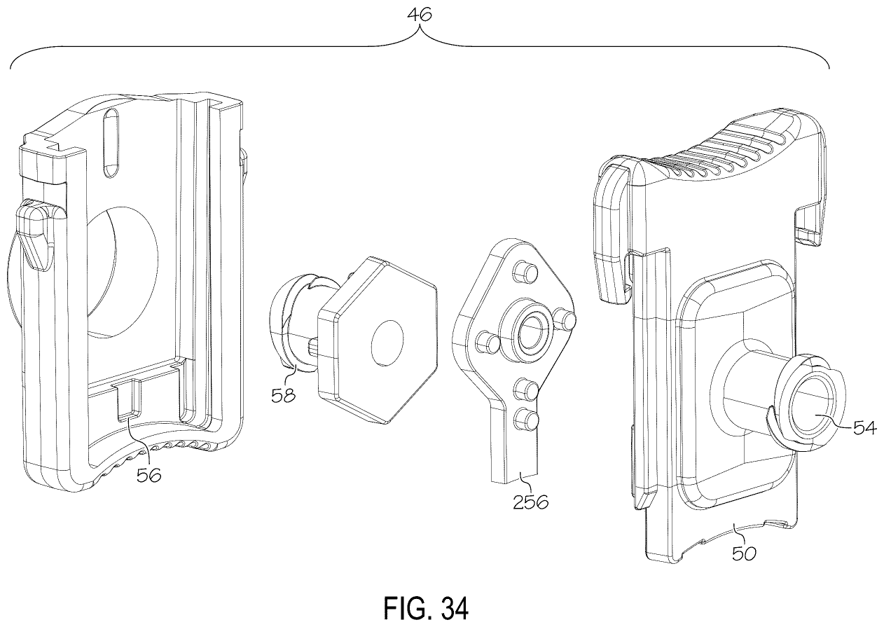

Referring to FIGS. 27-35, in additional aspects consistent with the present disclosure, a mixing system 40 can comprise a syringe coupler 46 that is configured to couple a first syringe 42 and a second syringe 44, wherein each of the first and second syringes has a respective male connector 45 (e.g., a male Luer lock connector). The displaceable member 50 can comprise a male extension 54 for engaging the male Luer lock connector of the second syringe 44. Accordingly, the male extension 54 can define at least one male thread at a distal end of the male extension 54 and an inner bore configured to receive therein at least a portion of a male projection of the male connector 45 of the second syringe 44. The guide member 56 can house a syringe engagement member (e.g., Luer lock member 58) within the central aperture 70, wherein the syringe engagement member (e.g., Luer lock member 58) is configured to engage a male connector (e.g., a male Luer lock connector) of the first syringe 42. Accordingly, the syringe engagement member (e.g., Luer lock member 58) can comprise a fitting having at least one thread at a distal end and an inner bore configured to receive at least a portion of a male projection of the male connector of the first syringe 42. In some aspects, the syringe engagement member (e.g., Luer lock member 58) can be rotatable within the guide member 56.

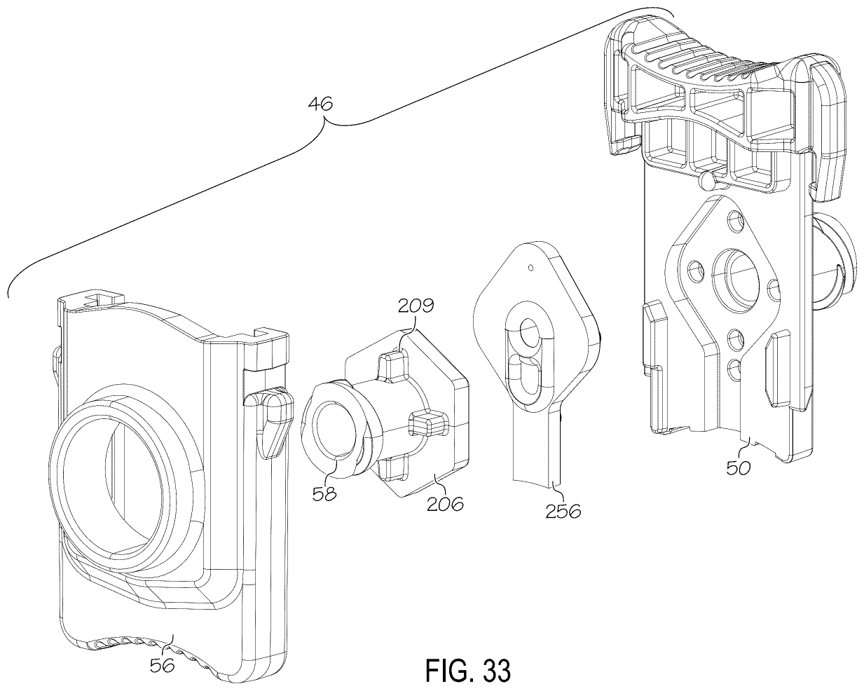

Referring to FIG. 33, the syringe engagement member (e.g., Luer lock member 58) can further comprise a flange 206. In some aspects, the syringe engagement member (e.g., Luer lock member 58) can further comprise a centering structure 209 that defines at least one bearing surface 84. The bearing surface(s) 84 can be configured to engage a surface defining the aperture 70 of the guide member 56. In this way, the centering structure 209 can center syringe engagement member (e.g., the Luer lock member 58) within the aperture 70 while permitting the syringe engagement member (e.g., Luer lock member 58) to rotate within the aperture 70 of the guide member 56. In some aspects, the centering structure 209 can comprise a plurality of radial projections (e.g., two, three, four, or more radial projections) that extend axially from the flange 206 toward the distal end of the syringe engagement member (e.g., Luer lock member 58). In other aspects, the centering structure 209 can comprise a continuous structure (e.g., that does not have discrete projections). It is contemplated that the bearing surface(s) 84 of the centering structure 209 can have a radial dimension that is substantially the same as that of the bearing surface 84 of the syringe engagement member (e.g., Luer lock member 58) for engaging a female Luer lock connector (e.g., as illustrated in FIG. 6A-6B). In this way, the syringe coupler 46 can be adapted for use with the connector of a particular syringe by selecting syringe engagement member (e.g., the Luer lock member 58) with the desired connector (e.g., male or female Luer lock connector).

Optionally, the syringe coupler 46 of the mixing system 40 shown in FIGS. 27-35 can include an interconnected sealing element 256, described herein with reference to FIGS. 16A-21.

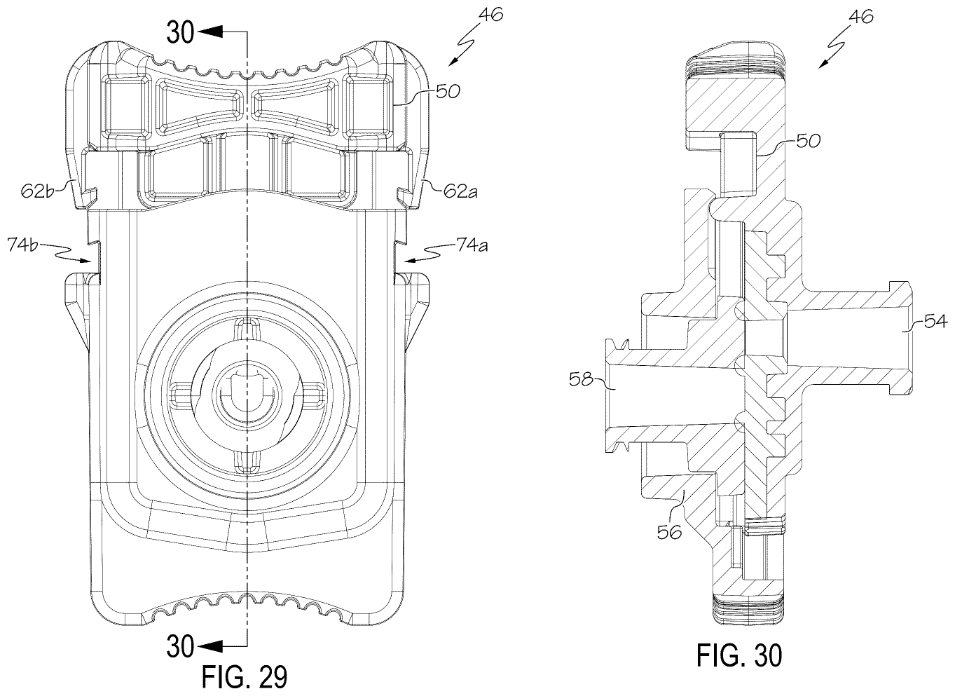

Referring to FIGS. 29-32, the displaceable member 50 can be displaceable relative to the guide member 56 so that the resilient projections 62a, 62b secure the displaceable member 50 in the second position within guide member 56 through engagement of said resilient projections 62a, 62b into recesses 74a, 74b located upon guide member 56 to prevent or inhibit the displaceable member from being returned to a first position.

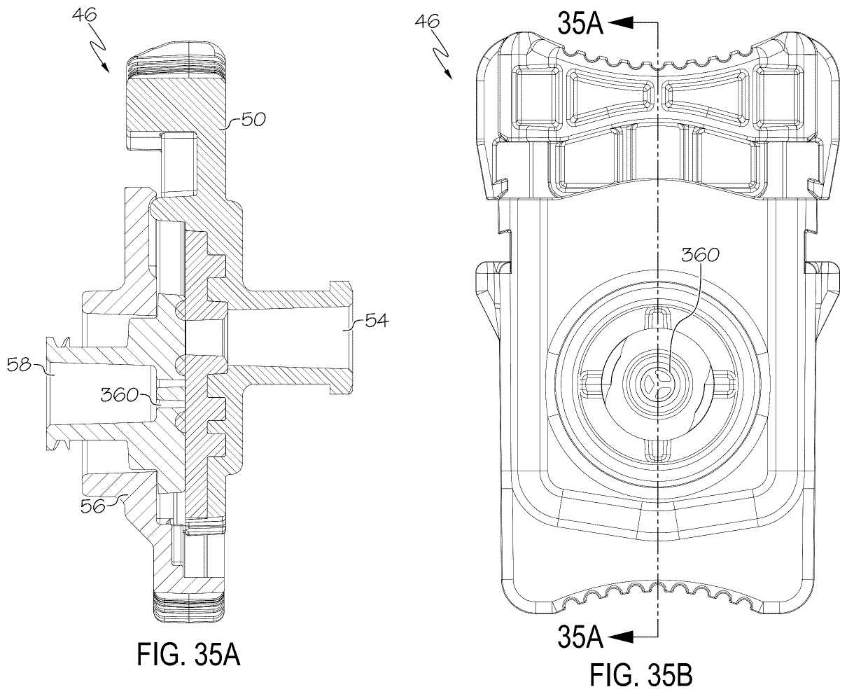

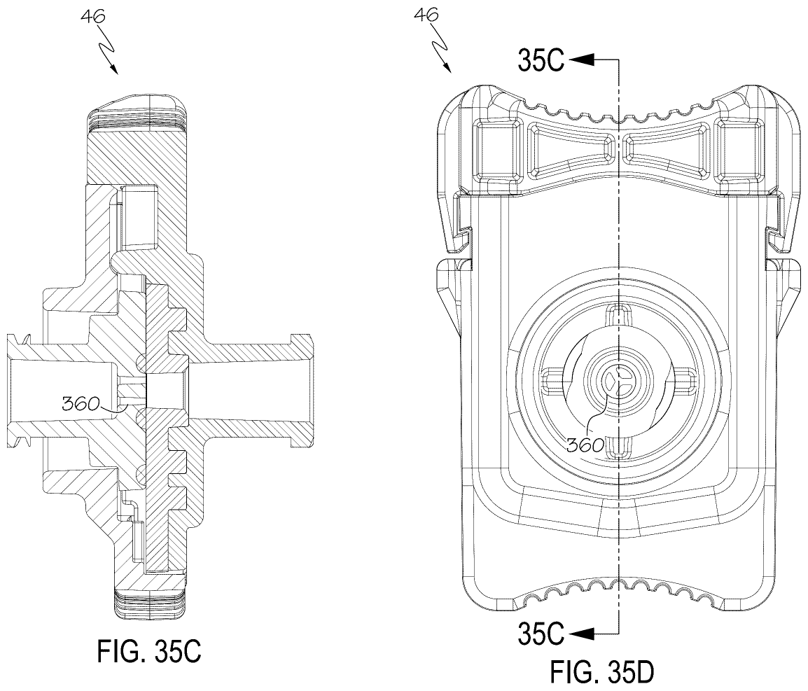

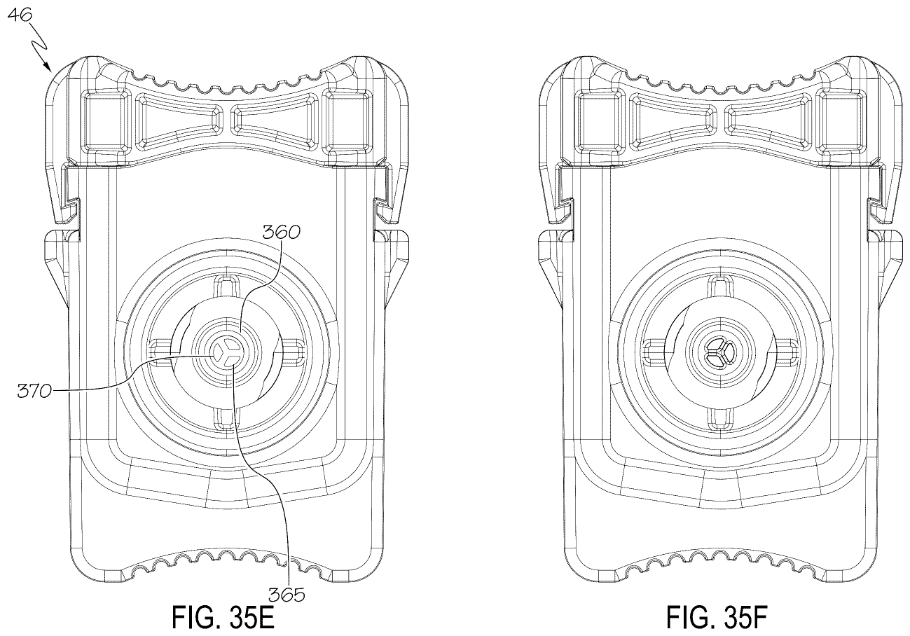

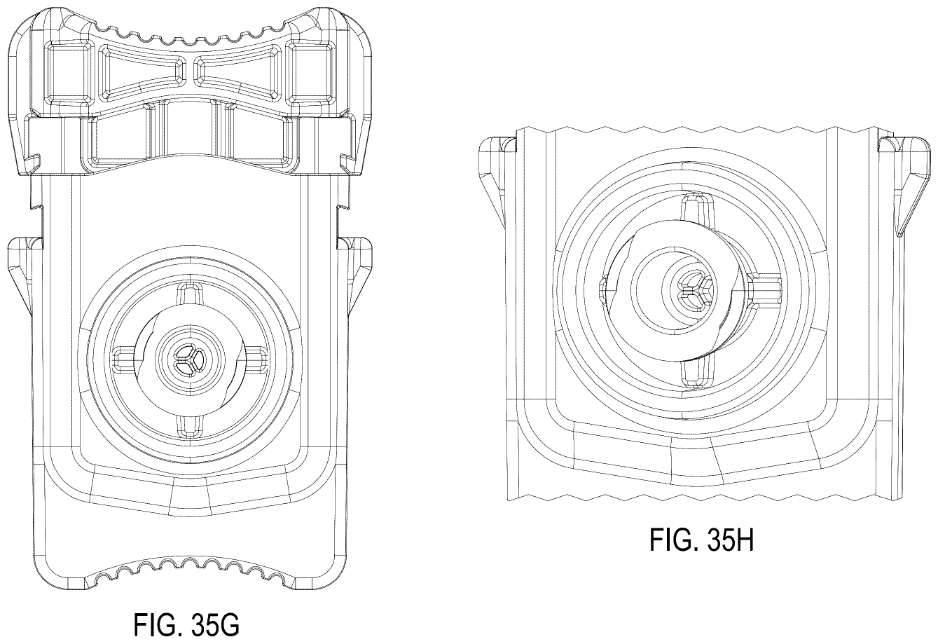

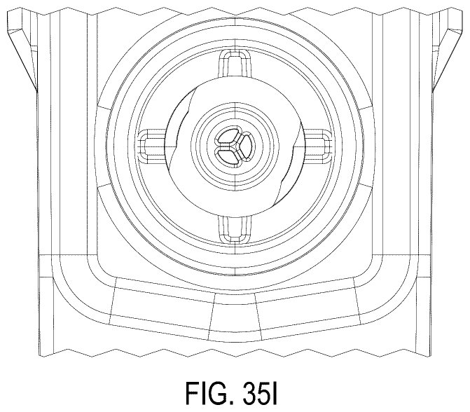

Referring to FIGS. 35A-35I, the syringe engagement member (e.g., Luer lock member 58) can further comprise a dividing structure 360 that extends across the flow path. In various aspects, the dividing structure 360 can define a plurality of openings 370 that extend through the dividing structure (to provide a reduced area of flow from one side of the dividing structure to the other). In some embodiments, the dividing structure 360 comprises a plurality of radially projecting arms 365 that intersect within the flow path (e.g., at a central axis of the flow path). As shown in FIG. 35, the plurality of radially projecting arms can be three arms. Optionally, in this aspect, the arms can be equally circumferentially spaced such that adjacent pairs of arms of the three arms can form 120 degree angles therebetween. However, in other aspects, it is contemplated that the arms can be unequally circumferentially spaced. In further aspects, it is contemplated that the plurality of radially projecting arms can be or comprise two, four, five, or more arms. In some embodiments, the plurality of openings comprises or consists of three openings. Although a specific example of a dividing structure is shown in FIGS. 35A-35I, it is contemplated that alternative dividing structures can be used. For example, it is contemplated that the plurality of openings can comprise any number of desired openings, such as, for example, 2, 3, 4, 5, 6, 7, 8, 9, 10, 11, 12, or more openings. Optionally, in some embodiments, the plurality of openings can comprise a plurality of perforations that are evenly or unevenly spaced about the area of the dividing structure. In other embodiments, it is contemplated that the plurality of openings can be defined within a grid structure. In still other embodiments, it is contemplated that the plurality of openings can comprise one or more radial projections that extend radially inwardly from an outer circumference of the dividing structure 60. In these embodiments, it is contemplated that the projections need not intersect or contact (e.g., with the ends of each projection being radially spaced from a central axis of the dividing structure 60), thereby providing a single continuous area for flow through the dividing structure. Optionally, the radial projections can be equally circumferentially spaced about the circumference of the dividing structure 60. Optionally, the radial projections can each have the same or substantially the same size and shape.

In some optional aspects, it is contemplated that a dividing structure such as described above can be positioned within the displaceable member 50. Thus, although not required, it is contemplated that the syringe coupler 46 disclosed herein can include at least one dividing structure (positioned within either the syringe engagement member (e.g., Luer lock member 58) or the displaceable member 50) or a plurality of dividing structures (with a respective dividing structure positioned within each of the syringe engagement member (e.g., Luer lock member 58) or the displaceable member 50). In use, it is contemplated that the dividing structure(s) can promote mixing of the formulations provided within each syringe as further disclosed herein. More particularly, as the formulations contact the solid (non-open) portions of the dividing structure and are forced to enter the reduced area of the flow path provided at the dividing structure, it is contemplated that mixing can be promoted or enhanced.

### B. Solid-Liquid Syringe Compositions and Methods of Use

According to the methods of administering the GnRH agonist or antagonist or a pharmaceutically acceptable salt thereof to a patient using the syringe device system, the method comprises subcutaneously administering at least one injection of a pharmaceutical composition comprising a GnRH agonist or antagonist or a pharmaceutically acceptable salt thereof, for a specified duration (e.g., once every month or once per month, once every three months or once per every three months, once every four months or once per every four months, or once every six months or once per every six months), to thereby reduce luteinizing hormone (LH) levels in a subject in need of LHRH reduction. The method in some aspects is useful in subjects having prostate cancer, pediatric patients 2 years of age or older having central precocious puberty (CPP), or subjects having hormone receptor-positive breast cancer, among other indications. According to certain methods of administering the GnRH agonist or antagonist or a pharmaceutically acceptable salt thereof to a patient with prostate cancer using the syringe device system disclosed herein, the method comprises subcutaneously administering at least one injection of a pharmaceutic composition comprising a unit dose of a GnRH agonist or antagonist or a pharmaceutically acceptable salt thereof to the patient to suppress the patient's serum testosterone level to less than or equal to 0.5 ng/ml.

i. Methods of Reconstituting and Administering Formulations

Prior to the administering, the pharmaceutical composition is reconstituted using the syringe device system comprising a first syringe barrel comprising a liquid formulation component (e.g., a polymer-solvent system) and a second syringe barrel comprising the GnRH agonist or antagonist or a pharmaceutically acceptable salt thereof (e.g., leuprolide acetate) in solid or liquid form, the first and second syringe barrels being interconnected via a syringe coupler comprising a displaceable seal, wherein the displaceable seal being operable to be axially displaced from a first position to a second position by a force applied to a plunger of the first syringe barrel, and wherein the first position comprises a position in which material transfer through the syringe coupler and between syringes is occluded, and the second position comprises a position in which at least a portion of the displaceable seal is positioned such that material transfer through the syringe coupler and between the syringes is permitted. The pharmaceutical composition is reconstituted by applying a force to a user-interface to move the displaceable seal from the first position to the second position and applying force to a plunger positioned in the first syringe barrel and a plunger positioned in the second syringe barrel in an alternating manner to mix contents of the first syringe barrel and the second syringe barrel.

According to the methods of activating the syringe device system, as disclosed herein, in one aspect where the first syringe barrel contains a polymer-solvent system and the second syringe barrel contains lyophilized leuprolide acetate, the user, after first allowing the pre-assembled syringe device system to equilibrate to room temperature and then removing it from its packaging, applies force to the user-interface portions 52 and 57 of the displaceable member 50 and the guide member 56, respectively, to activate the syringe coupler from the first closed position to the second open position. The user then applies a force to the first plunger disposed slidably within the first syringe to transfer the polymer-solvent system housed within the internal chamber of the first syringe barrel through the open, activated syringe coupler and into the internal chamber of the second syringe housing the lyophilized leuprolide acetate. Upon contact of the polymer-solvent system with the lyophilized leuprolide acetate, the leuprolide acetate will largely remain in suspension, thus requiring mixing with the polymer-solvent system to ensure that a homogeneous suspension is formed prior to administration. The user then applies a force to the second plunger disposed slidably within the second syringe to transfer the partially to fully mixed components back through the open syringe coupler and into the first syringe. The user will continue mixing the contents back and forth from the second and first syringes, for between about 15 seconds and two minutes. In some instances, mixing is contemplated as continuing for about 25 seconds, about 45 seconds, or in some instances for about 1 minute, equivalent to approximately 30-90 full back-and-forth cycles, and in some preferred embodiments, 60 full back-and-forth cycles, to ensure that the lyophilized leuprolide acetate is fully suspended within the polymer-solvent system. The fully formulated composition is subsequently displaced into the second syringe at a desired injection volume and administered formulation weight (both depending on duration and strength of dose). The user then disconnects the second syringe containing the therapeutic formulation from the syringe device by de-threading attachment to the male extension 54 upon the displaceable member 50 of the syringe connector. The user then attaches a needle, for example an 18G to 20G needle, to the distal dispensing outlet of the second syringe. The user then subcutaneously administers the formulation dose to the subject in need of treatment. This method is suitable for administering any of the specific solid-liquid dosage formulations described below.

ii. One-Month Formulation for Prostate Cancer

In some aspects, the syringe-to-syringe mixing system houses a pharmaceutical composition and the pharmaceutical composition comprises about 7.5 mg of leuprolide acetate as the active pharmaceutical ingredient, and N-methyl-2-pyrrolidone and a 50:50 poly(lactic acid-co-glycolic acid) (PLGA) copolymer having a weight average molecular weight from about 29 kDa to about 45 kDa and at least one terminal carboxylic acid end group as the liquid formulation component. In one aspect, the first syringe comprises N-methyl-2-pyrrolidone and a 50:50 poly(lactic acid-co-glycolic acid) (PLGA) copolymer having a weight average molecular weight from about 29 kDa to about 45 kDa and having at least one terminal carboxylic acid end group; and the second syringe comprises about 7.5 mg of leuprolide acetate. In a further aspect, the leuprolide acetate is present in the second syringe as a lyophilized powder. The term “weight average molecular weight,” unless otherwise specified, means a weight average molecular weight as measured by a conventional gel permeation chromatography (GPC) instrument (such as an Agilent 1260 Infinity Quaternary LC with Agilent G1362A Refractive Index Detector) utilizing polystyrene standards and tetrahydrofuran (THF) as the solvent.

In some instances, the amount of leuprolide or a pharmaceutically acceptable salt thereof in the delivered reconstituted product may be about 7.0 mg leuprolide free base equivalent. In some instances, the amount of leuprolide acetate in the delivered reconstituted product may be about 7.5 mg. As used herein, the term “free base equivalent” may refer to the conjugate base or deprotonated form of an amine containing compound or substance. For instance, about 7.0 mg of leuprolide represents the free base equivalent of about 7.5 mg of leuprolide acetate. In some instances, the amount of PLGA polymer in the delivered reconstituted product may be about 82.5 mg. In some instances, the amount of NMP in the delivered reconstituted product is about 160.0 mg. In some instances, the composition is mixed by pushing the contents back and forth between both syringes for a total of 60 cycles.

The fully formulated composition can be subsequently displaced into the second syringe at a final injection volume of about 0.25 mL and administered formulation weight of about 250 mg. The user then disconnects the second syringe containing the therapeutic formulation from the syringe device by de-threading attachment to the male extension 54 upon the displaceable member 50 of the syringe connector. The user then attaches a needle, for example an 18 G to 20 G needle, to the distal dispensing outlet of the second syringe. The user then subcutaneously administers the formulation dose to an adult male prostate cancer patient in need of treatment.

iii. Three-Month Formulation for Prostate Cancer

In some aspects, the syringe-to-syringe mixing system houses a pharmaceutical composition and the pharmaceutical composition comprises about 22.5 mg of leuprolide acetate as the active pharmaceutical ingredient and N-methyl-2-pyrrolidone, and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated as the liquid formulation component. In a further aspect, the first syringe comprises N-methyl-2-pyrrolidone and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated; and the second syringe comprises about 22.5 mg of leuprolide acetate.

In some instances, the amount of leuprolide or a pharmaceutically acceptable salt thereof in the delivered reconstituted product may be about 21.0 mg leuprolide free base equivalent. In some instances, the amount of leuprolide acetate in the delivered reconstituted product may be about 22.5 mg. In some instances, the amount of PLG polymer in the delivered reconstituted product may be about 158.6 mg. In some instances, the amount of NMP in the delivered reconstituted product is about 193.9 mg.

The fully formulated composition can be subsequently displaced into the second syringe at a final injection volume of about 0.375 mL and administered formulation weight of about 375 mg. The user then disconnects the second syringe containing the therapeutic formulation from the syringe device by de-threading attachment to the male extension 54 upon the displaceable member 50 of the syringe connector. The user then attaches a needle, for example an 18 G to 20 G needle, to the distal dispensing outlet of the second syringe. The user then subcutaneously administers the formulation dose to an adult male prostate cancer patient in need of treatment.

iv. Four-Month Formulation for Prostate Cancer

In some aspects, the syringe-to-syringe mixing system houses a pharmaceutical composition and the pharmaceutical composition comprises about 30 mg of leuprolide acetate as the active pharmaceutical ingredient and N-methyl-2-pyrrolidone, and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated as the liquid formulation component. In a further aspect, the first syringe comprises N-methyl-2-pyrrolidone and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated; and the second syringe comprises about 30 mg of leuprolide acetate.

In some instances, the amount of leuprolide or a pharmaceutically acceptable salt thereof in the delivered reconstituted product may be about 28.0 mg leuprolide free base equivalent. In some instances, the amount of leuprolide acetate in the delivered reconstituted product may be about 30.0 mg. According to this embodiment, the syringe device system comprises a syringe containing an amount of a polymer-solvent system comprising an amount of a biodegradable polymer, which in some instances is a poly(D,L-lactide-co-glycolide) (i.e. PLG) polymer formulation dissolved in a biocompatible solvent, which in some instances is NMP. In some instances, the biodegradable PLG polymer may comprise a lactide to glycolide ratio of about 75:25. In some instances, the PLG polymer may be initiated with hexanediol. In some instances, the PLG polymer may compromise a copolymer containing two primary hydroxyl end groups. In some instances, the PLG polymer has a weight average molecular weight range of about 15 kDa to about 21 kDa. In some instances, the amount of PLG polymer in the delivered reconstituted product may be about 211.5 mg. In some instances, the amount of NMP in the delivered reconstituted product is about 258.5 mg.

The fully formulated composition can be subsequently displaced into the second syringe at a final injection volume of about 0.5 mL and administered formulation weight of about 500 mg. The user then disconnects the second syringe containing the therapeutic formulation from the syringe device by de-threading attachment to the male extension 54 upon the displaceable member 50 of the syringe connector. The user then attaches a needle, for example an 18 G to 20 G needle, to the distal dispensing outlet of the second syringe. The user then subcutaneously administers the formulation dose to an adult male prostate cancer patient in need of treatment.

### V. Six-Month Formulation for Prostate Cancer

In some aspects, the syringe-to-syringe mixing system houses a pharmaceutical composition and the pharmaceutical composition comprises about 45 mg of leuprolide acetate as the active pharmaceutical ingredient and N-methyl-2-pyrrolidone, and an 85:15 poly(lactide-co-glycolide) (PLG) copolymer having a weight average molecular weight from about 20 kDa to about 26 kDa and one distal end group that is hydroxyl-terminated and the other distal end group that is either hydroxyl-terminated or ester-terminated as the liquid formulation component. In a further aspect, the first syringe comprises N-methyl-2-pyrrolidone and an 85:15 poly(lactide-co-glycolide) (PLG) copolymer having a weight average molecular weight from about 20 kDa to about 26 kDa and one distal end group that is hydroxyl-terminated and the other distal end group that is either hydroxyl-terminated or ester-terminated; and the second syringe comprises about 45 mg of leuprolide acetate.

In some instances, the PLG polymer may be initiated with hexanediol. In some instances, the PLG polymer may compromise a copolymer containing two primary hydroxyl end groups or alternatively one distal end group that is hydroxyl-terminated and the other distal end group that is either hydroxyl-terminated or ester-terminated. In some instances, the amount of PLG polymer in the delivered reconstituted product may be about 165 mg. In some instances, the amount of NMP in the delivered reconstituted product is about 165 mg.

The fully formulated composition can be subsequently displaced into the second syringe at a final injection volume of about 0.375 mL and administered formulation weight of about 375 mg. The user then disconnects the second syringe containing the therapeutic formulation from the syringe device by de-threading attachment to the male extension 54 upon the displaceable member 50 of the syringe connector. The user then attaches a needle, for example an 18 G to 20 G needle, to the distal dispensing outlet of the second syringe. The user then subcutaneously administers the formulation dose to an adult male prostate cancer patient in need of treatment.

vi. Three-Month Formulation for HR-Positive Breast Cancer

In yet another embodiment, a syringe device system comprises a composition, which when formulated according to the methods of using the syringe device system as described herein to intermix two separated components of the composition prior to administration, may be useful in suppressing ovarian function in a patient with HR-positive breast cancer. The composition may further be useful in suppressing one or more of the patient's estradiol (E2) level to less than 20 μg/mL, the patient's follicle stimulating hormone (FSH) level to less than 40 IU/L, and/or the patient's mean serum luteinizing hormone (LH) level. In some instances, the composition may be administered concurrently with one or more other therapeutic treatments for HR-positive breast cancer, including, but not limited to endocrine therapy, chemotherapy, and/or radiotherapy. In some instances, the composition is administered by subcutaneous injection about once every three months (once per three months). According to this embodiment, the syringe device system comprises a first syringe containing an amount of a polymer-solvent system comprising an amount of a biodegradable polymer, which in some instances is a poly(D,L-lactide-co-glycolide) (i.e. PLG) polymer formulation dissolved in a biocompatible solvent, which in some instances is NMP, and a second syringe containing an amount of a GnRH agonist or antagonist or a pharmaceutically acceptable salt thereof. In some instances, the syringe device system comprises a second syringe containing an amount of lyophilized leuprolide or a pharmaceutically acceptable salt thereof, such as lyophilized leuprolide acetate. In some instances, the amount of leuprolide or a pharmaceutically acceptable salt thereof in the delivered reconstituted product may be about 26 mg to about 30 mg, preferably 28 mg leuprolide free base equivalent. In some instances, the amount of leuprolide acetate in the delivered reconstituted product may be about 28 mg to about 32 mg, preferably 30 mg. In some instances, the biodegradable PLG polymer may comprise a lactide to glycolide ratio of about 70:30 to about 80:20, preferably about 75:25. In some instances, the PLG polymer may be initiated with hexanediol. In some instances, the PLG polymer may compromise a copolymer containing two primary hydroxyl end groups. In some instances, the PLG polymer may be initiated with dodecanol. In some instances, the PLG polymer may compromise a copolymer containing a hydroxyl end group and an ester end group. In some instances, the PLG polymer has a weight average molecular weight range of about 15 kDa to about 45 kDa, preferably about 17 kDa to about 21 kDa. In some instances, the amount of PLG polymer in the delivered reconstituted product may be about 166 mg. In some instances, the amount of NMP in the delivered reconstituted product is about 202 mg.

The fully formulated composition is subsequently displaced into the second syringe at a final injection volume of about 0.375 mL and administered formulation weight of about 375 mg to about 400 mg (e.g., 398 mg). The user then disconnects the second syringe containing the therapeutic formulation from the syringe device by de-threading attachment to the male extension 54 upon the displaceable member 50 of the syringe connector. The user then attaches a needle, for example a 18 G to 20 G needle, to the distal dispensing outlet of the second syringe. The user then subcutaneously administers the full formulation dose to an adult breast cancer patient in need of treatment.

vii. Formulation for Treatment of CPP

In yet another embodiment, the syringe device system comprises a composition, which when formulated according to the methods of using the syringe device system as described herein to intermix two separated components of the composition prior to administration, may be useful in the treatment of CPP in a pediatric patient 2 years of age or older, when administered by subcutaneous injection about once every six months (once per six months) to reduce the pediatric patient's peak stimulated blood serum LH concentration to a pre-pubertal concentration level of less than 4 IU/L. According to this embodiment, the syringe device system comprises a first syringe containing an amount of a polymer-solvent system comprising an amount of a biodegradable polymer, which in some instances is a poly(D,L-lactide-co-glycolide) (i.e. PLG) polymer formulation dissolved in a biocompatible solvent, which in some instances is NMP, and a second syringe containing an amount of a GnRH agonist or antagonist or a pharmaceutically acceptable salt thereof. In some instances, the syringe device system comprises a second syringe containing an amount of lyophilized leuprolide or a pharmaceutically acceptable salt thereof, such as lyophilized leuprolide acetate. In some instances, the amount of leuprolide or a pharmaceutically acceptable salt thereof in the delivered reconstituted product may be about 42.0 mg leuprolide free base equivalent. In some instances, the amount of leuprolide acetate in the delivered reconstituted product may be about 45.0 mg. In some instances, the biodegradable PLG polymer may comprise a lactide to glycolide ratio of about 85:15. In some instances, the PLG polymer may be initiated with hexanediol. In some instances, the PLG polymer may compromise a copolymer where both distal end groups are hydroxyl terminated. In some instances, the PLG polymer may be initiated with dodecanol. In some instances, the PLG polymer may compromise a copolymer containing a hydroxyl end group and an ester end group. In some instances, the PLG polymer has a weight average molecular weight range of about 20 kDa to about 26 kDa. In some instances, the amount of PLG polymer in the delivered reconstituted product may be about 165 mg. In some instances, the amount of NMP in the delivered reconstituted product is about 165 mg.

The fully formulated composition is subsequently displaced into the second syringe at a final injection volume of about 0.375 mL and administered formulation weight of about 375 mg to about 400 mg (e.g., 398 mg). The user then disconnects the second syringe containing the therapeutic formulation from the syringe device by de-threading attachment to the male extension 54 upon the displaceable member 50 of the syringe connector. The user then attaches a needle, for example a 18 G to 20 G needle, to the distal dispensing outlet of the second syringe. The user then subcutaneously administers the full formulation dose to a pediatric CPP patient in need of treatment.

### C. Liquid-Liquid Syringe Compositions and Methods of Use

The liquid-liquid compositions are administered similarly to that described above except in some instances, a male-male syringe coupler is used and the number of mixing cycles may vary from those discussed above.

i. Administering Liquid-Liquid Compositions

In general, prior to the administering, the pharmaceutical composition is present in the syringe device system comprising a first syringe barrel comprising a liquid formulation component (e.g., a polymer-solvent system) and a second syringe barrel comprising a liquid API component comprising the GnRH agonist or antagonist or a pharmaceutically acceptable salt thereof (e.g., leuprolide acetate and solvent, such as NMP), the first and second syringe barrels being interconnected via a syringe coupler comprising a displaceable seal, wherein the displaceable seal being operable to be axially displaced from a first position to a second position by a force applied to a plunger of the first syringe barrel, and wherein the first position comprises a position in which material transfer through the syringe coupler and between syringes is occluded, and the second position comprises a position in which at least a portion of the displaceable seal is positioned such that material transfer through the syringe coupler and between the syringes is permitted. The pharmaceutical composition is mixed by applying a force to a user-interface to move the displaceable seal from the first position to the second position and applying force to a plunger positioned in the second syringe barrel (the syringe barrel containing the liquid API component) and a plunger positioned in the first syringe barrel (the barrel containing the liquid formulation component) in an alternating manner to mix contents of the second syringe barrel and the first syringe barrel. In contrast to the initial mixing step deployed for Solid-Liquid Compositions described above, the initial mixing step for Liquid-Liquid Compositions described herein comprises first applying force to the second plunger disposed slidably within the second syringe to transfer the liquid API component (e.g., solvent and API) housed within the internal chamber of the second syringe barrel through the open, activated syringe coupler and into the internal chamber of the first syringe housing the liquid formulation component (e.g., polymer-solvent system).

According to the methods of activating the syringe device system, as disclosed herein, in one aspect where the liquid formulation component is a polymer-solvent system and where the liquid API component is leuprolide in NMP, the user, after first allowing the pre-assembled syringe device system to equilibrate to room temperature and then removing it from its packaging, applies force to the user-interface portions 52 and 57 of the displaceable member 50 and the guide member 56, respectively, to activate the syringe coupler from the first closed position to the second open position. The user then applies a force to the second plunger disposed slidably within the second syringe to transfer the liquid API component (e.g., NMP and leuprolide acetate) housed within the internal chamber of the second syringe barrel through the open, activated syringe coupler and into the internal chamber of the first syringe housing the polymer-solvent system. Upon contact of the leuprolide acetate-NMP with the polymer-solvent system, the leuprolide acetate will largely remain in suspension, thus requiring mixing with the polymer-solvent system to ensure that a homogeneous suspension is formed prior to administration. The user then applies a force to the first plunger disposed slidably within the first syringe to transfer the partially to fully mixed components back through the open syringe coupler and into the second syringe. The user will continue mixing the contents back and forth from the first and second syringes, in some instances for 30 mixing cycles or fewer, 25 mixing cycles or fewer, or 20 mixing cycles or fewer, to ensure that the leuprolide acetate is fully homogenized within the polymer-solvent system. The fully formulated composition is subsequently displaced into the syringe which can be operably removed from the activated syringe connector (i.e. into the second syringe which originally contained the liquid API component prior to activation and subsequent mixing) at a desired injection volume and administered formulation weight (both depending on duration and strength of dose). The user then disconnects the second syringe containing the therapeutic formulation from the syringe device by de-threading attachment to the male extension 54 upon the displaceable member 50 of the syringe connector. The user then attaches a needle, for example an 18 G to 20 G needle, to the distal dispensing outlet of the second syringe. The user then subcutaneously administers the formulation dose to the subject in need of treatment. This method is suitable for administering any of the specific liquid-liquid dosage formulations described below.

In one aspect, the method of reducing lutenizing hormone (LH) levels in a subject in need of LHRH reduction, comprises: providing a disclosed syringe-to-syringe mixing system, wherein the first syringe comprises N-methyl-2-pyrrolidone (NMP) and a poly(lactide-co-glycolide) (PLG) or a poly(lactic acid-co-glycolic acid) (PLGA) copolymer, wherein the second syringe comprises N-methyl-2-pyrrolidone (NMP) and leuprolide acetate; positioning the displaceable member in the second position to form the fluid flow path; mixing, through the fluid flow path, the contents of the first syringe and the second syringe to form a pharmaceutical composition; and administering the pharmaceutical composition to the subject via subcutaneous injection through the second syringe after disconnecting the second syringe from the syringe connector which remains attached to the first syringe.

Advantageously, the described liquid-liquid formulations require in some instances fewer mixing cycles relative to the liquid-solid formulations described above. Thus, in some aspects, mixing, through the fluid flow path, comprises cyclically mixing contents between the first and second syringes through a number of mixing cycles. In some aspects, preparing the injectable composition comprises mixing the contents of the first and second syringe for 30 or fewer mixing cycles. In a further aspect, the method comprises mixing the contents of the first and second syringe for 35 or fewer mixing cycles, 25 or fewer mixing cycles, or 20 or fewer mixing cycles.

ii. Liquid-Liquid Formulations

The product/compositions/formulations disclosed herein comprise leuprolide acetate (sometimes abbreviated “LA”) (i.e., the API) in polymer-based drug delivery system. Generally, the disclosed products comprise a first container (e.g., a syringe) comprising biodegradable polymer dissolved in a biocompatible solvent (also referred to as a “liquid formulation component” herein); and a second container (e.g., a syringe) comprising a drug solution comprising LA dissolved in the biocompatible solvent (also referred to as a “liquid API component” herein), wherein, for the entire shelf life of the product, the drug solution remains a solution and the drug solution concentration is less than 45% w/w LA in the biocompatible solvent. In other words, regardless of the starting drug solution concentration when the pharmaceutical product is first produced or manufactured, over the full shelf life of the product, when solvent may, depending on the container configuration and/or materials, leak or evaporate in small quantities, the drug solution concentration must always be less than 45% w/w LA in the biocompatible solvent. In aspects of the disclosure, for the entire shelf life of the product, the drug solution remains a solution and the drug solution concentration is less than 44% w/w LA, or less than 43% w/w LA, or less than 42% w/w LA, or less than 41% w/w LA, or less than 40% w/w LA, less than 39% w/w LA, less than 38% w/w LA, less than 37% w/w LA, or less than 36% w/w LA, in the biocompatible solvent. In some embodiments, the drug solution concentration, at any or all times in the life of the product (e.g., at any or all times from when the product is first produced or manufactured through expiration, or end of shelf life, of the product), is 20% to 45% w/w LA or 22% to 36% w/w LA, e.g., 20% LA, 21% LA, 22% LA, 23% LA, 24% LA, 25% LA, 26% LA, 27% LA, 28% LA, 29% LA, 30% LA, 31% LA, 32% LA, 33% LA, 34% LA, 35% LA, 36% LA, 37% LA, 38% LA, 39% LA, 40% LA, 41% LA, 42% LA, 43% LA, 44% LA, or 45% LA.

In embodiments, the disclosed products comprise a first container comprising biodegradable polymer dissolved in a biocompatible solvent; and a second container comprising a drug solution comprising LA dissolved in the biocompatible solvent, wherein, for the entire shelf life of the product, the drug solution remains a solution and the drug solution concentration is no more than 45% w/w/LA, 44% w/w LA, 43% w/w LA, 42% w/w LA, 41% w/w LA, 40% w/w LA, 39% w/w LA, 38% w/w LA, 37% w/w LA, 36% w/w LA, 35% w/w LA (e.g., about 25% LA), in the biocompatible solvent. Similarly to the discussion above, in other words, regardless of the starting drug solution concentration when the pharmaceutical product is first produced or manufactured, over the full shelf life of the product, when solvent may, depending on the container configuration and/or materials, leak or evaporate in small quantities, the drug solution concentration may always be no more than 45% or 44% w/w LA in the biocompatible solvent.

Accordingly, in any of the above embodiments, in aspects of the disclosure, the drug solution concentration at the time of manufacture or production of the pharmaceutical product (i.e., the targeted drug solution concentration, which may be a range according to product specifications) is no more than about 45% w/w LA, about 44% w/w LA, about 43% w/w LA, about 42% w/w LA, about 41% w/w LA, about 40% w/w LA, about 39% w/w LA, about 38% w/w LA, about 37% w/w LA, about 36% w/w LA, or about 35% w/w LA (e.g., about 25% LA) in the biocompatible solvent. In aspects, the drug solution concentration at the time of manufacture or production of the pharmaceutical product is no more than about 20% to about 45% w/w LA in the biocompatible solvent, or no more than about 20% to about 40% w/w LA, no more than about 22% to about 36% w/w LA. In further aspects, the drug solution concentration at the time of manufacture or production of the pharmaceutical product is no more than about 22% to about 39% w/w LA, no more than about 22% to about 38% w/w LA, no more than about 22% to about 37% w/w LA, or no more than about 22% to about 36% w/w LA. In aspects, the drug solution concentration of the pharmaceutical product when the pharmaceutical product is manufactured, is between about 5% to about 45% w/w LA in the biocompatible solvent, between about 10% to about 45% w/w LA in the biocompatible solvent, between about 20% to about 45% w/w LA in the biocompatible solvent, or between about 20% to about 42% w/w LA in the biocompatible solvent, or between about 22% to about 36% w/w LA in the biocompatible solvent. In some embodiments, the drug solution concentration at the time of manufacture or production of the pharmaceutical product is less than about 45% w/w LA, less than about 44% w/w LA, less than about 43% w/w LA, less than about 42% w/w LA in the biocompatible solvent, or less than about 41% w/w LA in the biocompatible solvent, or less than about 40% w/w LA in the biocompatible solvent, or less than about 39% w/w LA in the biocompatible solvent, or less than about 38% w/w LA in the biocompatible solvent, or less than about 37% w/w LA in the biocompatible solvent, or less than about 36% w/w LA in the biocompatible solvent, or less than about 35% w/w LA in the biocompatible solvent, or less than about 30% w/w LA in the biocompatible solvent, or less than about 30% in the biocompatible solvent, or less than about 25% w/w in the biocompatible solvent (e.g., about 25% LA).

The drug solution concentration at the time of manufacture or production of the pharmaceutical product (i.e., the targeted drug solution concentration) can vary and may range from about 5% to about 45% LA by weight in the biocompatible solvent, including any whole number percent to any other whole number percent within the range of from about 5 percent to about 45 percent by weight. Regardless of the drug solution concentration at the time of manufacture or production of the pharmaceutical product, the drug solution concentration remains a solution and is less than 45% w/w LA in the biocompatible solvent over the shelf life of the pharmaceutical product, and/or the drug solution concentration remains a solution and is no more than 45% or 44% w/w LA in the biocompatible solvent over the shelf life of the pharmaceutical product.

In embodiments of the disclosure, the shelf life of the pharmaceutical product is about 12 months, about 18 months, about 24 months, about 30 months, about 36 months, about 42 months, about 48 months, about 54 months or about 60 months storage at 2-8° C. In embodiments, the shelf life of the pharmaceutical product is about 12 months, about 18 months, about 24 months, about 30 months, about 36 months, about 42 months, about 48 months, about 54 months or about 60 months storage at room temperature. In embodiments, the shelf life of the pharmaceutical product is about 6 months, about 12 months, about 18 months, about 24 months, about 30 months, about 36 months, about 42 months, about 48 months, about 54 months or about 60 months storage at up to 25° C. In other embodiments, the drug solution concentration is less than 45% w/w/LA, less than 44%, less than 43%, less than 42%, less than 41%, less than 40%, less than 39%, less than 38%, less than 37%, less than 36% or less than 35%, w/w LA in the biocompatible solvent at about 24 months storage at 2-8° C. In embodiments, the drug solution concentration is no more than 44%, or no more than 43%, or no more than 42%, or no more than 41%, or no more than 40%, or no more than 39%, or no more than 38%, or no more than 37%, or no more than 36%, or no more than 35%, w/w LA in the biocompatible solvent at about 24 months storage at 2-8° C. In still other embodiments, the drug solution concentration is less than 45% w/w/LA, less than 44%, less than 43%, less than 42%, less than 41%, less than 40%, less than 39%, less than 38%, less than 37%, less than 36% or less than 35%, w/w LA in the biocompatible solvent, at about 6 months storage at 25° C. In still other embodiments, the drug solution concentration is no more than 44%, or no more than 43%, or no more than 42%, or no more than 41%, or no more than 40%, or no more than 39%, or no more than 38%, or no more than 37%, or no more than 36%, or no more than 35%, w/w LA in the biocompatible solvent at about 6 months storage at 25° C.

In other embodiments, the drug solution concentration at about 24 months storage at 2-8° C. is 20% to about 45% w/w LA in the biocompatible solvent, or no more than about 20% to about 40% w/w LA, or no more than about 22% to about 36% w/w LA. In further aspects, the drug solution concentration at about 24 months storage at 2-8° C. is no more than about 22% to about 39% w/w LA, or no more than about 22% to about 38% w/w LA, or no more than about 22% to about 37% w/w LA, or no more than about 22% to about 36% w/w LA. In other embodiments, the drug solution concentration at about 6 months storage at 25° C. is 20% to about 45% w/w LA in the biocompatible solvent, or no more than about 20% to about 40% w/w LA, or no more than about 22% to about 36% w/w LA. In further aspects, the drug solution concentration at about 6 months storage at 25° C. is no more than about 22% to about 39% w/w LA, or no more than about 22% to about 38% w/w LA, or no more than about 22% to about 37% w/w LA, or no more than about 22% to about 36% w/w LA.

In various aspects, the present disclosure provides extended-release, injectable pharmaceutical compositions comprising LA, a biocompatible and biodegradable polymer, a biocompatible solvent, and optionally one or more additives. All such compositions are contemplated for administration to a subject to treat a disease or condition as disclosed herein. In various aspects, the products/compositions of the disclosure are contemplated for use to treat prostate cancer, including advanced prostate cancer; or to treat central precocious puberty. Further, such products/compositions are contemplated for administration to a subject to reduce luteinizing hormone (LH) levels in a subject in need of LHRH reduction, to reduce serum testosterone levels, and to suppress ovarian function in a subject with HR+ breast cancer palliatively treat cancer in a male with HR+ breast cancer. Further contemplated the pharmaceutical products disclosed herein, can include an amount effective to: (1) treat other diseases or conditions (e.g., including as a palliative therapy) including, but not limited to, hormone-related endometrial cancer, hormone-related ovarian cancer, hormone-related cervical cancer, endometriosis, and fibroids; (2) reducing the levels in a subject of various hormones by GnRH pathways (testosterone, estrogen/estradiol, follicle stimulating hormone (FSH), etc.), (3) suppressing functions associated with hormones in the GnRH pathways, such as suppressing ovarian function in a subject with HR+ breast cancer; and (4) blocking or suppression of hormones in the GnRH pathway for other purposes, such as to prevent or delay puberty in a transgender individual.

The drug solution comprising the LA dissolved in the biocompatible solvent (as in a solution or suspension), at administration is then combined/mixed with the biodegradable polymer dissolved also in solvent (as in a solution or suspension).

As provided for herein, the delivered amount in the first and/or second container (i.e. syringe(s)) is the amount (or the contribution) that is delivered to the subject after homogeneous mixing of the contents of the first and second containers occurs. In various aspects the first and/or second container may be a syringe as disclosed herein. In one embodiment, the first and second containers are a first and second syringe, respectively. In one embodiment, the first and second syringes are pre-filled with the polymer-solvent solution (first syringe) and the LA-solvent solution (second syringe) and are further provided pre-connected in a kit or package.

In some embodiments, the present disclosure provides a pharmaceutical product wherein the biodegradable polymer is a PLG copolymer having at least one hydroxyl end group, wherein the molar ratio of the lactide to glycolide monomers in the copolymer is about 50:50, about 75:25, or about 85:15, and the biocompatible solvent in the first container and the second container is NMP.

iii. One-Month Liquid-Liquid Formulation for Prostate Cancer

In some embodiments, the present disclosure provides a pharmaceutical product wherein the first syringe delivers about 75 mg to about 90 mg acid-initiated 50:50 PLG copolymer and from about 100 mg to about 170 mg of NMP (e.g., 125 mg to 155 mg); the second syringe delivers about 6.7 mg to about 8.3 mg LA (e.g., about 7.5 mg LA) and from about 5 mg to about 40 mg NMP; and wherein when contents of the first and second syringes are mixed, the resulting composition is an extended-release composition for subcutaneous injection into a subject that upon injection into the subject, forms an in situ depot that releases the LA over a time period of about 1 month. In one aspect, the second container delivers about 7.5 mg LA and about 15.0 mg NMP. In another aspect, the first syringe delivers about 82.5 mg acid-initiated 50:50 PLG copolymer and about 145 mg NMP. In one aspect, the first and second syringes deliver the amounts of LA, NMP, and polymer as shown for any of the illustrative formulations in Table 11.

iv. Three-Month Liquid-Liquid Formulation for Prostate Cancer

In some embodiments, the present disclosure provides a pharmaceutical product wherein the first syringe delivers about 150 mg to about 170 mg of 1,6-hexane-diol initiated 75:25 PLG copolymer (e.g., 155 mg to 165 mg) and from about 90 mg to about 170 mg of NMP; the second syringe delivers about 20 to about 25 mg LA (e.g., about 22.5 mg) and from about 25 mg to about 105 mg NMP; and wherein when contents of the first and second syringes are mixed, the resulting composition is an extended-release composition for subcutaneous injection into a subject that upon injection into the subject, forms an in situ depot that releases the LA over a time period of about 3 months. In one aspect, the second syringe delivers about 22.5 mg LA and about 43.9 mg NMP. In still another aspect, the first syringe delivers about 158.6 mg 1,6-hexane-diol initiated PLG copolymer and about 150 mg NMP. In one aspect, the second syringe delivers about 22.5 mg LA and about 71.3 mg NMP. In still another aspect, the first syringe delivers about 158.6 mg 1,6-hexane-diol initiated PLG copolymer and about 122.6 mg NMP. In one aspect, the second syringe delivers the amounts of LA and NMP and the first syringe delivers the amounts of polymer and NMP as shown for any of the illustrative formulations in Table 12.

### V. Three-Month Liquid-Liquid Formulation for Breast Cancer

In some embodiments, the present disclosure provides a pharmaceutical product wherein the first syringe delivers about 145 mg to about 185 mg of 1,6-hexane-diol initiated 75:25 PLG copolymer and from about 80 mg to about 200 mg of NMP; the second syringe delivers about 27 mg to about 33 mg LA (e.g., about 30 mg) and from about 55 mg to about 120 mg NMP, or in one aspect from about 35 mg to about 135 mg NMP; and wherein when contents of the first and second syringes are mixed, the resulting composition is an extended-release composition for subcutaneous injection into a subject that upon injection into the subject, forms an in situ depot that releases the LA over a time period of about 3 months. In one aspect, the second syringe delivers about 30.0 mg LA and about 58.3 mg NMP. In yet another aspect, the first syringe delivers about 166 mg 1,6-hexane-diol initiated PLG copolymer and about 143.7 mg NMP. In one aspect, the second syringe delivers the amounts of LA and NMP and the first syringe delivers the amounts of polymer and NMP as shown for any of the illustrative formulations in Table 13.

vi. Four-Month Liquid-Liquid Formulation for Prostate Cancer

In some embodiments, the present disclosure provides a pharmaceutical product wherein the first syringe delivers about 190 mg to about 230 mg of 1,6-hexane-diol initiated 75:25 PLG copolymer and from about 80 mg to about 225 mg of NMP; the second syringe delivers about 27 mg to about 33 mg LA and from about 30 mg to about 135 mg NMP; and wherein when contents of the first and second syringes are mixed, the resulting composition is an extended-release composition for subcutaneous injection into a subject that upon injection into the subject, forms an in situ depot that releases the LA over a time period of about 4 months. In one aspect, the second syringe delivers about 30.0 mg LA and about 58.5 mg NMP. In still another aspect, the first syringe delivers about 211.5 mg 1,6-hexane-diol initiated PLG copolymer and about 200 mg NMP. In one aspect, the second syringe delivers about 30.0 mg LA and about 95.7 mg NMP. In still another aspect, the first syringe delivers about 211.5 mg 1,6-hexane-diol initiated PLG copolymer and about 162.2 mg NMP. In one aspect, the second syringe delivers the amounts of LA and NMP and the first syringe delivers the amounts of polymer and NMP as shown for any of the illustrative formulations in Table 14.

vii. Six-Month Liquid-Liquid Formulation for Prostate Cancer or CPP

In some embodiments, the present disclosure provides a pharmaceutical product wherein the first syringe delivers about 145 mg to about 185 mg of 1,6-hexane-diol initiated 85:15 PLG copolymer and from about 100 mg to about 170 mg of NMP; the second syringe delivers 40 mg to about 50 mg LA (e.g., about 45 mg) and from about 55 mg to about 95 mg NMP; and wherein when contents of the first and second syringes are mixed, the resulting composition is an extended-release composition for subcutaneous injection into a subject that upon injection into the subject, forms an in situ depot that releases the LA over a time period of about 6 months. In one aspect, the second syringe delivers about 45.0 mg LA and about 86 mg NMP. In still another aspect, the first container delivers about 165 mg 1,6-hexane-diol initiated PLG copolymer and about 135 mg NMP. In one aspect, the second syringe delivers the amounts of LA and NMP and the first syringe delivers the amounts of polymer and NMP as shown for any of the illustrative formulations in Table 15.

viii. Additional Exemplary Formulations

Additional exemplary embodiments include the following liquid-liquid products suitable for administration to subject once per every one month, three months, four months, or six months:

i) A pharmaceutical product suitable for administration to a subject once per every one month, where the biodegradable polymer is a PLG polymer (e.g., a 50:50 PLG or PLGH polymer), the biocompatible solvent is NMP; the first syringe is formulated to deliver 100 mg to 170 mg NMP (e.g., 125 mg to 155 mg) and 75 mg to 90 mg of the PLG polymer, and the second syringe is formulated to deliver 7.5 mg leuprolide acetate and 5 mg to 40 mg NMP.

ii) A pharmaceutical product suitable for administration to a subject once per every three months, where the biodegradable polymer is a PLG polymer (e.g., a 75:25 PLG polymer), the biocompatible solvent is NMP; the first syringe is formulated to deliver 90 mg to 170 mg NMP and 150 mg to 170 mg of the PLG polymer (e.g., 155 mg to 165 mg, or 155 to 160 mg), and the second syringe is formulated to deliver 22.5 mg leuprolide acetate and 25 mg to 105 mg NMP.

iii) A pharmaceutical product suitable for administration to a subject once per every three months, where the biodegradable polymer is a PLG polymer (e.g., a 75:25 PLG polymer), the biocompatible solvent is NMP; the first syringe is formulated to deliver 80 mg to 200 mg NMP and 145 mg to 185 mg of the PLG polymer, and the second syringe is formulated to deliver 30 mg leuprolide acetate and 35 mg to 135 mg NMP, and in one aspect, 55 mg to 120 mg NMP.

iv) A pharmaceutical product suitable for administration to a subject once per every four months, where the biodegradable polymer is a PLG polymer (e.g., a 75:25 PLG polymer), the biocompatible solvent is NMP; the first syringe is formulated to deliver 80 mg to 225 mg NMP and 190 mg to 230 mg of the PLG polymer, and the second syringe is formulated to deliver 30 mg leuprolide acetate and 30 mg to 135 mg NMP.

v) A pharmaceutical product suitable for administration to a subject once per every six months, where the biodegradable polymer is a PLG polymer (e.g., an 85:15 PLG polymer), the biocompatible solvent is NMP; the first syringe is formulated to deliver 100 mg to 170 mg NMP and 145 mg to 185 mg of the PLG polymer, and the second syringe is formulated to deliver 45 mg leuprolide acetate and 55 mg to 95 mg NMP.

In some embodiments, the present disclosure provides an injectable extended release composition which when injected into a subject delivers about 0.43 mL total volume of the composition having about 45.0 mg LA; about 165.0 mg 85:15 PLG copolymer having at least one hydroxyl end group, and about 221 mg NMP, wherein the composition is formulated for subcutaneous administration about once per six months.

In some embodiments, unreacted lactide and/or glycolide monomers in the polymers or copolymers within the pharmaceutical product and/or the final composition are less than about 1.0 wt %, less than about 0.5 wt %, than about 0.4 wt %, less than about 0.3 wt %, less than about 0.2 wt % and less than about 0.1 wt %.

Various features and embodiments of a pre-connected syringe-to-syringe device and system and methods of using the pre-connected syringe-to-syringe device and system have been provided herein. It will be recognized, however, that various features are not necessarily specific to certain embodiments and may be provided on any one or more embodiments. The present disclosure and embodiments provided herein are not mutually exclusive and may be combined, substituted, and omitted. The scope of the invention(s) provided herein is thus not limited to any particular embodiment, drawing, or particular arrangement of features.

### D. Treatment Methods, Uses, and Administration

The methods of this disclosure are used in the treatment of diseases or conditions including prostate cancer, including advanced prostate cancer. Still further, the methods of this disclosure are used in reducing serum testosterone levels in a subject to a level below 20 ng/dL, below 10 ng/dL or lower. In one aspect, the compositions disclosed herein reduce lutenizing hormone (LH) in a subject in need of LHRH, which in one aspect, is reduced to a level less than about 4 IU/L. In another aspect the reduction of LH levels treats prostate cancer. In yet another aspect, the reduction of LH levels suppresses ovarian function in a subject with hormone receptor positive (HR+) breast cancer in a subject.

Further, the methods of this disclosure are used in suppressing ovarian function in a subject with hormone-receptor positive (HR+) breast cancer. In one aspect, the hormone receptor positive breast cancer is pre-menopausal breast cancer. In one aspect, the hormone receptor positive breast cancer is peri-menopausal breast cancer. In one aspect, the hormone receptor positive breast cancer is estrogen receptor (ER) positive breast cancer. In one aspect, the compositions disclosed herein suppress a subject's estradiol (E2) production to a level less than about 20 μg/mL, less than about 15 μg/mL, less than about 10 μg/mL, less than about 5 μg/mL, less than about 4 μg/ml, less than about 3 μg/mL, or less than about 2 μg/mL. In one aspect, the E2 production level is reduced to about 2.7 pg/mL. In still another aspect, the compositions disclosed herein suppresses the breast cancer subject's follicle stimulating hormone (FSH) to a level less than about 40 IU/L. In yet another aspect, the compositions disclosed herein suppresses the breast cancer subject's leutenizing hormone (LH) to a level less than about 4 IU/L.

Still further, the methods of this disclosure are used in treating endometriosis or uterine fibroids, or other hormone-related cancers including hormone-related endothelial cancer or hormone-related ovarian cancer.

The methods of this disclosure are used in the treatment of central precocious puberty (CPP). CPP is defined by early sexual development prompted by production and release of gonadotropins and/or sex steroids from normal endogenous sources including the hypothalamus or pituitary. Aberrations in gonadotropin and/or sex hormone concentration levels in children with CPP can result from various sources, including, but not limited to, physical injury, infection, genetic disease, or associated tumors. CPP caused by a genetic or undetermined pathology is classified to be idiopathic in nature, while CPP caused by a central nervous system (CNS) tumor and/or lesion is classified as organic in nature. CPP is accompanied by advanced bone age, accelerated growth velocity, and Hypothalamic-Pituitary-Gonadal-axis activation. In one aspect, the compositions disclosed herein reduce the blood serum LH concentration in a subject having CPP to a pre-pubertal concentration levels of <4 IU/L.

The methods and/or uses disclosed herein comprise subcutaneously administering to the subjects disclosed herein, the disclosed extended-release injectable compositions subsequent to mixing the contents of the first container (such as a syringe) comprising the biodegradable polymer disclosed herein dissolved in a biocompatible solvent disclosed herein, and the second container (such as a syringe) comprising the drug solution (DS) comprising LA dissolved in the biocompatible solvent disclosed herein, wherein the DS concentration is no more than 45% w/w LA in the biocompatible solvent. Upon injection of the extended-release pharmaceutical composition into the body and contact of the composition with a bodily fluid, the solvent dissipates, forming a drug reservoir or depot. The resulting depot will release the LA, over a desired extended time period. In various embodiments, the LA is released into a subject/patient, for a period of at least about 30 days or longer, at least about 60 days or longer, at least about 90 days or longer, at least about 120 days or longer, at least about 150 days or longer, or 180 days or longer. In still other embodiments, the LA is released into a subject/patient, for a period of at least about one month or longer, at least about two months or longer, at least about three months or longer, at least about four months or longer, at least about five months or longer, or six months or longer. In still other embodiments, the LA is released into a subject/patient for a period of at least about four weeks or longer, at least about eight weeks or longer, at least about twelve weeks or longer, at least about sixteen weeks or longer, at least about 20 weeks or longer, or at least about 24 weeks or longer.

The extended-release composition may be administered to the patient/subject about every 30 days (e.g., once every 30 days), about every 60 days, about every 90 days, about every 120 days, about every 150 days or about every 180 days. In another aspect, the extended-release composition may be administered to the patient/subject about every 1 month (e.g., about once every month), about every 2 months, about every 3 months, about every 4 months, about every 5 months or about every 6 months. In another aspect, the extended-release composition may be administered to the patient/subject about every four weeks, about every eight weeks, about every twelve weeks, about every sixteen weeks, about every 20 weeks, or about every 24 weeks.

The disclosed extended-release compositions may be administered to a subject/patient once in a dosing period with varying durations, or a non-variable duration, between dosing periods (e.g., one month, 2 months, 3 months, 4 months, 5 months, or 6 months). In one aspect, there may be a single loading dose or alternatively two or three loading doses, followed by maintenance doses having a defined interval between doses. The loading dose(s) may provide a different amount of LA API to the patient, or the same amount of LA API to the patient, as compared to the maintenance dose. Dosing may be provided alone or in combination with other drugs and may continue as long as required for effective treatment of the disease state or disorder.

In some embodiments, the composition is terminally sterilized by irradiation (such as e-beam, Gamma irradiation, or X-ray). In yet another aspect, the composition is sterile filtered.

In some embodiments, the extended-release composition(s) disclosed herein is administered as a monotherapy to patients. The therapeutic methods of this embodiment may reduce or eliminate one or more symptoms of the disease and/or condition disclosed herein. In other embodiments, the long-acting composition may be administered as a combination therapy, such as with chemotherapeutics, radiation therapy, surgery, endocrine therapies such as selective estrogen receptor modulators (SERMs; such as tamoxifen, toremifene, raloxifene, ospemifene, and bazedoxifene), selective estrogen receptor degraders (SERDs; such as fulvestrant), aromatase inhibitors (Als; such as anastrozole, letrozole, exemestane, vorozole, formestane, and fadrozole); mammalian target of rapamycin (mTOR) inhibitors; such as temsirolimus, sirolimus, everolimus, and ridaforolimus); Phosphatidylinositol 3-kinases inhibitors (PI-3 kinase or PI3K; such as alpelisib, idelalisib, and buparlisib); cyclin-dependent kinases 4 and 6 inhibitors (CDK4/6 inhibitors; such as abemaciclib, palbociclib, and ribociclib); other LHRH agonists (such as other salts of leuprolide, gonadorelin, goserelin, histrelin, nafarelin, buserelin, and triptorelin and their pharmaceutically acceptable salts thereof), immuno-therapy, and gene therapy.

The disclosed products/compositions may be provided as a part of a delivery system comprising a syringe system, wherein the product/composition is contained within syringes. Accordingly, such delivery systems are within the scope of the present disclosure.

For the pre-filled syringe delivery system for administration of the product/composition, the first container is a first syringe comprising the polymer-solvent solution (also known as a liquid formulation component herein) and the second container is a second syringe comprising the drug-solvent solution (also known as a liquid API component herein). In one aspect of this system, the first syringe and the second syringe are coupled together to mix the contents of the first and second syringe. In the prefilled syringe system disclosed herein, the polymer solution of the first syringe is mixed with the drug solution of the second syringe to form an extended-release composition for subcutaneous injection into a subject.

For solid-liquid syringe compositions, it can be advantageous to start with the plunger of the first syringe and move the polymer-solvent contents into the second syringe which can comprise leuprolide acetate, e.g., lyophilized leuprolide acetate, continue with the back-and-forth mixing cycles described above, disconnect the second syringe from the first syringe, and administer the reconstituted pharmaceutical composition. Conversely, with liquid-liquid formulations, it was surprisingly discovered that mixing can be improved if the user starts with the plunger of the second syringe (containing, e.g., leuprolide acetate and NMP), and moves the contents of this syringe into the first syringe containing the polymer-solvent, followed by mixing back and forth through a sufficient number of mixing cycles, i.e., back and forth between the first and second syringes, (e.g., 35 or fewer, 30 or fewer, 25 or fewer, or 20 or fewer back-and-forth cycles), followed by disconnection of the second syringe from the first syringe and administration of the pharmaceutical composition.

A plurality of mixing cycles is performed to mix the contents of the first syringe with the contents of the second syringe to visual and/or substantial homogeneity with either the liquid-liquid or solid-liquid formulations, though surprisingly, fewer are generally needed with the liquid-liquid formulations. Homogeneity can mean a visually uniform depot for injection or wherein the homogeneity of the formulation is substantially similar at the beginning, middle, and end of syringe as measured via an in-unit content uniformity assay, or wherein homogeneity of a between-unit delivered dose content uniformity is substantially similar. In one aspect, the contents of the first syringe and the second syringe require, for liquid-liquid formulations, 35 cycles or fewer cycles of mixing, 30 or fewer cycles of mixing, 25 or fewer cycles of mixing, 20 or fewer cycles of mixing, 15 or fewer cycles of mixing, 10 or fewer cycles of mixing, or 5 or fewer cycles of mixing. In one aspect, the contents of the first syringe and second syringe require 5-30 cycles of mixing, 10-30 cycles of mixing, 20-30 cycles of mixing, 5-20 cycles of mixing, 10-20 cycles of mixing, or 5-10 cycles of mixing, including for example 20 cycles, 25 cycles or 30 cycles. Solid-liquid formulations typically require more mixing cycles such as 60 cycles or less, including for example 60 cycles. As disclosed elsewhere herein, one complete mixing cycle is defined as one complete push of the plunger for the first syringe and one complete push of the plunger for the second syringe, regardless of whether the first push begins with the first or the second plunger. The formulation may be administered by manual injection or automated though a syringe with, for example, a 16 to 24 gauge needle, or an 18 to 22 gauge needle, or an 18 to 20 gauge needle with standard, or thin wall, or extra thin walls. Also contemplated herein is a kit comprising a prefilled syringe system disclosed herein and instructions for mixing and administration.

### E. Exemplary Aspects

Aspect 1: A sealing element for packaging and sealing of mixing syringes, the sealing element comprising: an elastomeric member comprising a first side and an opposing second side; the first side and the second side each comprising a planar portion; at least one aperture provided through the elastomeric member and forming a fluid flow path through the elastomeric member; the first side of the elastomeric member comprising a raised projection with a first portion that surrounds the at least one aperture; and wherein the raised projection is at least partially surrounded by the planar portion.

Aspect 2: The sealing element of Aspect 1, wherein the elastomeric member comprises a pharmaceutically acceptable thermoplastic.

Aspect 3: The sealing element of Aspects 1 or 2, wherein the second side of the elastomeric member comprises a second raised projection.

Aspect 4: The sealing element of Aspect 3, wherein the raised projection of the first side and the second raised projection of the second side are each operable to provide a fluidic seal.

Aspect 5: The sealing element of any one of Aspects 1-4, further comprising at least one rigid housing member operable to receive the sealing element and wherein the at least one rigid housing member comprises a syringe-receiving portion.

Aspect 6: The sealing element of any one of Aspects 1-5, wherein the raised projection further comprises a second portion that extends adjacent to the first portion and comprises a planar seal operable to prevent fluid flow.

Aspect 7: A syringe-to-syringe mixing system comprising: a first syringe comprising a hollow body, the hollow body having a proximal end and a distal dispensing end; a second syringe comprising a hollow body, the second syringe comprising a distal dispensing end; the first syringe and the second syringe each comprising a barrel and a plunger for applying pressure to a syringe content; a valve assembly that is operable to receive the first syringe and the second syringe, and wherein the valve assembly comprises at least one resilient member that is biased toward a locked position; wherein the valve assembly comprises a displaceable member comprising a user-interface and a guide member, wherein the displaceable member is slidable relative to the guide member, and wherein the user-interface is operable to receive a force from a user and transmit the force to a displaceable seal provided within the displaceable member; wherein the displaceable seal comprises a planar portion, a protrusion, and a flow path, and wherein the displaceable seal is moveable in a direction substantially perpendicular to a longitudinal axis of at least one of the first syringe and the second syringe; a selectively rotatable member operable to receive at least one of the first syringe and the second syringe; wherein the valve assembly comprises a first position wherein a fluid flow between the first syringe and the second syringe through the displaceable seal is fully occluded, and a second position in which fluid is allowed to flow through the displaceable seal and between the first syringe and the second syringe; wherein the displaceable member is provided in communication with the selectively rotatable member when the displaceable member is in the first position, and wherein the displaceable member is displaced to a position that allows rotation of the selectively rotatable member when the displaceable member is in the second position.

Aspect 8: The syringe-to-syringe mixing system of Aspect 7, wherein at least one of the first syringe and the second syringe is moveable with the valve assembly.

Aspect 9: The syringe-to-syringe mixing system of Aspect 7 or 8, wherein the first syringe and the second syringe each comprise a liquid component.

Aspect 10: The syringe-to-syringe mixing system of any one of Aspects 7-9, wherein at least one of the first syringe and the second syringe comprises leuprolide acetate.

Aspect 11: The syringe-to-syringe mixing system of any one of Aspects 7-10, wherein the selectively rotatable member comprises a threaded member operable to receive at least one of the first syringe and the second syringe.

Aspect 12: The syringe-to-syringe mixing system of any one of Aspects 7-11, wherein the protrusion of the displaceable seal comprises at least one of an annular projection and an upstanding ridge.

Aspect 13: The syringe-to-syringe mixing system of any one of Aspects 7-12, wherein the syringe-to-syringe mixing system houses a pharmaceutical composition and the pharmaceutical composition comprises about 7.5 mg of leuprolide acetate as the active pharmaceutical ingredient and N-methyl-2-pyrrolidone and a 50:50 poly(lactic acid-co-glycolic acid) (PLGA) copolymer having a weight average molecular weight from about 29 kDa to about 45 kDa and at least one terminal carboxylic acid end group.

Aspect 14: The syringe-to-syringe mixing system of any one of Aspects 7-12, wherein the syringe-to-syringe mixing system houses a pharmaceutical composition and the pharmaceutical composition comprises about 22.5 mg of leuprolide acetate as the active pharmaceutical ingredient and N-methyl-2-pyrrolidone, and a 75:25 poly(lactide-co-glycolide) (PLG) copolymer having a weight average molecular weight from about 17 kDa to about 21 kDa and one distal end group that is hydroxyl-terminated and the other distal end group that is either hydroxyl-terminated or ester-terminated.

Aspect 15: The syringe-to-syringe mixing system of any one of Aspects 7-12, wherein syringe-to-syringe mixing system houses a pharmaceutical composition and the pharmaceutical composition comprises about 30 mg of leuprolide acetate as the active pharmaceutical ingredient and N-methyl-2-pyrrolidone, and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated.

Aspect 16: The syringe-to-syringe mixing system of any one of Aspects 7-12, wherein syringe-to-syringe mixing system houses a pharmaceutical composition and the pharmaceutical composition comprises about 45 mg of leuprolide acetate as the active pharmaceutical ingredient and N-methyl-2-pyrrolidone, and an 85:15 poly(lactide-co-glycolide) (PLG) copolymer having a weight average molecular weight from about 20 kDa to about 26 kDa and one distal end group that is hydroxyl-terminated and the other distal end group that is either hydroxyl-terminated or ester-terminated as the liquid formulation component.

Aspect 17: A syringe-to-syringe mixing system comprising: a combined syringe coupler and valve assembly that is operable to receive a first syringe and a second syringe; wherein the valve assembly comprises a first portion and a second portion that are displaceable relative to one another between at least a first position and a second position; the first portion and the second portion each comprising a syringe receiving portion and an internal flow port; wherein the second portion comprises a sealing element that is displaceable with and in fixed relative position to the second syringe; the sealing element comprising a flow path and at least one of a projection and an upstanding portion positioned circumferentially around a central axis of the flow path, and wherein the sealing element is moveable between the first position and the second position, the first position comprising a sealed position and the second position comprising a mixing position, wherein in the first position, the internal flow port of the second portion is offset from the internal flow port of the first portion, and wherein in the second position, the internal flow port of the second portion is aligned or substantially aligned with and in fluid communication with the internal flow port of the first portion; and wherein with the sealing element in the first position, the at least one of a projection and the upstanding portion provides a contact seal against the first portion to prevent leakage of fluid from the internal flow port of the first portion to the internal flow port of the second portion and to prevent leakage of fluid from the internal flow port of the second portion to the internal flow port of the first portion, and wherein with the sealing element in the second position, the at least one of a projection and the upstanding portion provides a contact seal against the first portion that surrounds the internal flow port of the first portion to prevent leakage of fluid passing between the internal flow ports of the first and second portions to allow mixing.

Aspect 18: The syringe-to-syringe mixing system of Aspect 17, wherein the combined syringe coupler and valve assembly comprises a user-interface operable to receive a force from a user and transmit the force to the sealing element.

Aspect 19: The syringe-to-syringe mixing system of Aspect 17 or 18, wherein the sealing elements comprises a resilient sealing element provided with and at least partially recessed within the second portion.

Aspect 20: The syringe-to-syringe mixing system of any one of Aspects 17-19, wherein the combined syringe coupler and valve assembly comprises a rotatable Luer lock member that is free to rotate when the combined syringe coupler and valve assembly is provided in the second position.

Aspect 21: The syringe-to-syringe mixing system of any one of Aspects 17-20, further comprising a first syringe coupled to the first portion and a second syringe coupled to the second portion, and wherein at least one of the first syringe and the second syringe comprises at least one of leuprolide acetate and a pharmaceutically acceptable salt thereof.

Aspect 22: The syringe-to-syringe mixing system of any one of Aspects 17-21, wherein at least one of the first portion and the second portion comprises a resilient projection biased toward a locked position.

Aspect 23: The syringe-to-syringe mixing system of any one of Aspects 17-22, wherein the syringe-to-syringe mixing system houses a pharmaceutical composition and the pharmaceutical composition comprises about 7.5 mg of leuprolide acetate as the active pharmaceutical ingredient and N-methyl-2-pyrrolidone and a 50:50 poly(lactic acid-co-glycolic acid) (PLGA) copolymer having a weight average molecular weight from about 29 kDa to about 45 kDa and at least one terminal carboxylic acid end group.

Aspect 24: The syringe-to-syringe mixing system of any one of Aspects 17-22, wherein the syringe-to-syringe mixing system houses a pharmaceutical composition and the pharmaceutical composition comprises about 22.5 mg of leuprolide acetate as the active pharmaceutical ingredient and N-methyl-2-pyrrolidone, and a 75:25 poly(lactide-co-glycolide) (PLG) copolymer having a weight average molecular weight from about 17 kDa to about 21 kDa and end groups that are hydroxyl-terminated.

Aspect 25: The syringe-to-syringe mixing system of any one of Aspects 17-22, wherein syringe-to-syringe mixing system houses a pharmaceutical composition and the pharmaceutical composition comprises about 30 mg of leuprolide acetate as the active pharmaceutical ingredient and N-methyl-2-pyrrolidone, and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated.

Aspect 26: The syringe-to-syringe mixing system of any one of Aspects 17-22, wherein syringe-to-syringe mixing system houses a pharmaceutical composition and the pharmaceutical composition comprises about 45 mg of leuprolide acetate as the active pharmaceutical ingredient and N-methyl-2-pyrrolidone, and an 85:15 poly(lactide-co-glycolide) (PLG) copolymer having a weight average molecular weight from about 20 kDa to about 26 kDa and one distal end group that is hydroxyl-terminated and the other distal end group that is either hydroxyl-terminated or ester-terminated.

Aspect 27: A syringe coupler configured to couple to a first syringe and a second syringe, each of the first and second syringes having a respective male connector, the syringe coupler comprising: a guide member defining a syringe receiving portion, the syringe receiving portion defining an internal flow port, wherein the guide member is configured to engage the male connector of the second syringe to establish fluid communication between the internal flow port of the syringe receiving portion and the second syringe; a displaceable member axially slidable relative to the guide member about and between a first position and a second position; and a syringe engagement member at least partly housed within the guide member, the syringe engagement member defining an internal flow port, wherein the syringe engagement member is configured to engage the male connector of the first syringe to establish fluid communication between the internal flow port of the syringe engagement member and the first syringe; and a sealing element defining a flow port and at least one of a projection and an upstanding portion positioned circumferentially around a central axis of the flow port, wherein the sealing element is coupled to and in a fixed position relative to the displaceable member, wherein: when the displaceable member is in the first position, the internal flow port of the syringe receiving portion of the displaceable member is offset from the internal flow port of the syringe engagement member, and the at least one of the projection or the upstanding portion of the sealing element forms a contact seal with the syringe engagement member that prevents leakage of fluid from the internal flow port of the syringe receiving portion of the displaceable member to the internal flow port of the syringe engagement member and prevents leakage of fluid from the internal flow port of the syringe engagement member to the internal flow port of the syringe receiving portion of the displaceable member; and when the displaceable member is in the second position, the internal flow ports of the syringe receiving portion and the syringe engagement member and the flow port of the sealing element are aligned or substantially aligned to define a flow path, and the at least one of the projection or the upstanding portion of the sealing element provides a contact seal with the syringe engagement member that surrounds the internal flow port of the syringe engagement member to prevent leakage of fluid passing between the internal flow ports of the syringe receiving portion of the displaceable member and the syringe engagement member.

Aspect 28: The syringe coupler of Aspect 27, wherein the syringe receiving portion is configured to engage a male Luer lock connector, wherein the syringe engagement member is configured to engage a male Luer lock connector.

Aspect 29: The syringe coupler of Aspect 27 or 28, wherein the syringe engagement member further comprises a dividing structure that partially occludes the flow path to promote mixing within the flow path.

Aspect 30: The syringe coupler of Aspect 27 or 28, wherein the syringe engagement member further comprises a dividing structure that extends across the flow path to define a plurality of through-openings.

Aspect 31: The syringe coupler of Aspect 30, wherein the dividing structure comprises a plurality of radially projecting arms that intersect within the flow path at a central axis of the flow path.

Aspect 32: The syringe coupler of Aspect 31, wherein the plurality of radially projecting arms are three arms.

Aspect 33: The syringe coupler of Aspect 32, wherein adjacent pairs of arms of the three arms form 120 degree angles therebetween.

Aspect 34: The syringe coupler of Aspect 30, wherein the plurality of through-openings comprises three through-openings.

Aspect 35: A syringe-to-syringe mixing system comprising: first and second syringes; and a syringe coupler as in any one of Aspects 27-34.

Aspect 36: The syringe-to-syringe mixing system of any one of Aspects 7-12, 21, or 27, wherein the first syringe comprises N-methyl-2-pyrrolidone and a 50:50 poly(lactic acid-co-glycolic acid) (PLGA) copolymer having a weight average molecular weight from about 29 kDa to about 45 kDa and having at least one terminal carboxylic acid end group; and the second syringe comprises about 7.5 mg of leuprolide acetate.

Aspect 37: The syringe-to-syringe mixing system of any one of Aspects 7-12, 17-22, or 35, wherein the first syringe comprises N-methyl-2-pyrrolidone and a 75:25 poly(lactide-co-glycolide) (PLG) copolymer having a weight average molecular weight from about 17 kDa to about 21 kDa and one distal end group that is hydroxyl-terminated and the other distal end group that is either hydroxyl-terminated or ester-terminated; and the second syringe comprises about 22.5 mg of leuprolide acetate.

Aspect 38: The syringe-to-syringe mixing system of any one of Aspects 7-12, 17-22, or 35, wherein the first syringe comprises N-methyl-2-pyrrolidone and a 75:25 poly(lactide-co-glycolide) (PLG) copolymer having a weight average molecular weight from about 15 kDa to about 21 kDa and one distal end group that is hydroxyl-terminated and the other distal end group that is either hydroxyl-terminated or ester-terminated; and the second syringe comprises about 30 mg of leuprolide acetate.

Aspect 39: The syringe-to-syringe mixing system of any one of Aspects 7-12, 17-22, or 35, wherein the first syringe comprises N-methyl-2-pyrrolidone and an 85:15 poly(lactide-co-glycolide) (PLG) copolymer having a weight average molecular weight from about 20 kDa to about 26 kDa and one distal end group that is hydroxyl-terminated and the other distal end group that is either hydroxyl-terminated or ester-terminated; and the second syringe comprises about 45 mg of leuprolide acetate.

Aspect 40: The syringe-to-syringe mixing system of any one of Aspects 36-39, wherein the leuprolide acetate is present in the second syringe as a lyophilized powder.

Aspect 41: The syringe-to-syringe mixing system of any one of Aspects 36-39, wherein the second syringe further comprises N-methyl-2-pyrrolidone (NMP), and the leuprolide acetate is dissolved in the NMP.

Aspect 42: The syringe-to-syringe mixing system of any one of Aspects 7-12, 17-22, or 35, or 41, wherein the first syringe is formulated to deliver 100 mg to 170 mg of N-methyl-2-pyrrolidone and 75 mg to 90 mg of a 50:50 poly(lactic acid-co-glycolic acid) (PLGA) copolymer; and the second syringe is formulated to deliver 7.5 mg leuprolide acetate and 5 mg to 40 mg of N-methyl-2-pyrrolidone.

Aspect 43: The syringe-to-syringe mixing system of any one of Aspects 7-12, 17-22, or 35, or 41, wherein the first syringe is formulated to deliver 90 mg to 170 mg of N-methyl-2-pyrrolidone and 150 mg to 170 mg of a 75:25 poly(lactide-co-glycolide) (PLG) copolymer; and the second syringe is formulated to deliver 22.5 mg leuprolide acetate and 25 mg to 105 mg of N-methyl-2-pyrrolidone.

Aspect 44: The syringe-to-syringe mixing system of any one of Aspects 7-12, 17-22, or 35, or 41, wherein the first syringe is formulated to deliver 80 mg to 200 mg of N-methyl-2-pyrrolidone and 145 mg to 185 mg of a 75:25 poly(lactide-co-glycolide) (PLG) copolymer; and the second syringe is formulated to deliver 30 mg leuprolide acetate and 55 mg to 120 mg of N-methyl-2-pyrrolidone or 35 mg to 135 mg of N-methyl-2-pyrrolidone.

Aspect 45: The syringe-to-syringe mixing system of any one of Aspects 7-12, 17-22, or 35, or 41, wherein the first syringe is formulated to deliver 80 mg to 225 mg of N-methyl-2-pyrrolidone and 190 mg to 230 mg of a 75:25 poly(lactide-co-glycolide) (PLG) copolymer; and the second syringe is formulated to deliver 30 mg leuprolide acetate and 30 mg to 135 mg of N-methyl-2-pyrrolidone.

Aspect 46: The syringe-to-syringe mixing system of any one of Aspects 7-12, 17-22, or 35, or 41, wherein the first syringe is formulated to deliver 100 mg to 170 mg of N-methyl-2-pyrrolidone and 145 mg to 185 mg of a 85:15 poly(lactide-co-glycolide) (PLG) copolymer; and the second syringe is formulated to deliver 45 mg leuprolide acetate and 55 mg to 95 mg of N-methyl-2-pyrrolidone.

Aspect 47: A kit comprising the syringe-to-syringe mixing system of any one of Aspects 36-46 together with instructions for mixing and administration.

Aspect 48: The syringe-to-syringe mixing system of any one of Aspects 36-46, for use in a method of reducing luteinizing hormone (LH) levels in a subject in need of LHRH reduction.

Aspect 49: Use of the syringe-to-syringe mixing system of any one of Aspects 36-46 in the manufacture of a medicament for use in a method of reducing luteinizing hormone (LH) levels in a subject in need of LHRH reduction.

Aspect 50: A method of reducing luteinizing hormone (LH) levels in a subject in need of LHRH reduction, the method comprising: providing the syringe-to-syringe mixing system of Aspect 35, wherein the first syringe comprises N-methyl-2-pyrrolidone and a poly(lactide-co-glycolide) (PLG) or a poly(lactic acid-co-glycolic acid) (PLGA) copolymer, wherein the second syringe comprises leuprolide acetate; positioning the displaceable member in the second position to form the fluid flow path; mixing, through the fluid flow path, the contents of the second syringe and the first syringe to form a reconstituted pharmaceutical composition; and administering the reconstituted pharmaceutical composition to the subject via subcutaneous injection through the second syringe after disconnecting the second syringe from the first syringe.

Aspect 51: The method of Aspect 50, wherein positioning the displaceable member in the second position comprises moving the displaceable member from the first position to the second position.

Aspect 52: A method of reducing lutenizing hormone (LH) levels in a subject in need of LHRH reduction, the method comprising: providing the syringe-to-syringe mixing system of Aspect 21, wherein the first syringe comprises N-methyl-2-pyrrolidone and a poly(lactide-co-glycolide) (PLG) or a poly(lactic acid-co-glycolic acid) (PLGA) copolymer, wherein the second syringe comprises leuprolide acetate; moving the sealing element from the first position to the second position to form a fluid flow path between the first syringe and the second syringe; mixing, through the fluid flow path, the contents of the second syringe and the first syringe to form a reconstituted pharmaceutical composition; and administering the reconstituted pharmaceutical composition to the subject via subcutaneous injection through the second syringe after disconnecting the second syringe from the first syringe.

Aspect 53: A method of reducing lutenizing hormone (LH) levels in a subject in need of LHRH reduction, the method comprising: providing the syringe-to-syringe mixing system of any one of Aspects 7-12, wherein the first syringe comprises N-methyl-2-pyrrolidone and a poly(lactide-co-glycolide) (PLG) or a poly(lactic acid-co-glycolic acid) (PLGA) copolymer, wherein the second syringe comprises leuprolide acetate; moving the valve assembly from the first position to the second position to allow fluid to flow through the displaceable seal between the first syringe and the second syringe; mixing, through the displaceable seal, the contents of the second syringe and the first syringe to form a reconstituted pharmaceutical composition; and administering the reconstituted pharmaceutical composition to the subject via subcutaneous injection through the second syringe after disconnecting the second syringe from the first syringe.

Aspect 54: The method of any one of Aspects 50-53, wherein mixing, through the fluid flow path, comprises cyclically mixing contents between the second and first syringes through a number of mixing cycles.

Aspect 55: The method of any one of Aspects 50-54, wherein the reconstituted pharmaceutical composition comprises: about 7.5 mg of leuprolide acetate; N-methyl-2-pyrrolidone; and a 50:50 poly(lactic acid-co-glycolic acid) (PLGA) copolymer having a weight average molecular weight from about 29 kDa to about 45 kDa, and having at least one terminal carboxylic acid end group.

Aspect 56: The method of Aspect 55, further comprising repeating the positioning, mixing, and administering steps once per month.

Aspect 57: The method of any one of Aspects 50-54, wherein the reconstituted pharmaceutical composition comprises: about 22.5 mg of leuprolide acetate; N-methyl-2-pyrrolidone; and a 75:25 poly(lactide-co-glycolide) (PLG) copolymer having a weight average molecular weight from about 17 kDa to about 21 kDa and having one distal end group that is hydroxyl-terminated and another distal end group that is either hydroxyl-terminated or ester-terminated.

Aspect 58: The method of Aspect 57, further comprising repeating the positioning, mixing, and administering steps once every three months.

Aspect 59: The method of any one of Aspects 50-54, wherein the reconstituted pharmaceutical composition comprises: about 30 mg of leuprolide acetate; N-methyl-2-pyrrolidone; and a 75:25 poly(lactide-co-glycolide) (PLG) copolymer having a weight average molecular weight from about 15 kDa to about 21 kDa and having one distal end group that is hydroxyl-terminated and another distal end group that is either hydroxyl-terminated or ester-terminated.

Aspect 60: The method of Aspect 59, further comprising repeating the positioning, mixing, and administering steps once every four months.

Aspect 61: The method of Aspect 59, further comprising repeating the positioning, mixing, and administering steps once every three months.

Aspect 62: The method of any one of Aspects 50-54, wherein the reconstituted pharmaceutical composition comprises: about 45 mg of leuprolide acetate; and N-methyl-2-pyrrolidone; and an 85:15 poly(lactide-co-glycolide) (PLG) copolymer having a weight average molecular weight from about 20 kDa to about 26 kDa and having one distal end group that is hydroxyl-terminated and another distal end group that is either hydroxyl-terminated or ester-terminated.

Aspect 63: The method of Aspect 62, further comprising repeating the positioning, mixing, and administering steps once per every six months.

Aspect 64: The method of any one of Aspects 50-63, wherein the subject has prostate cancer, is a pediatric patient 2 years of age or older having central precocious puberty (CPP), or has hormone receptor-positive breast cancer.

Aspect 65: A method of reducing luteinizing hormone (LH) levels in a subject in need of LHRH reduction, the method comprising: providing the syringe-to-syringe mixing system of Aspect 35, wherein the first syringe comprises N-methyl-2-pyrrolidone and a poly(lactide-co-glycolide) (PLG) or a poly(lactic acid-co-glycolic acid) (PLGA) copolymer, wherein the second syringe comprises N-methyl-2-pyrrolidone and leuprolide acetate; positioning the displaceable member in the second position to form the fluid flow path; mixing, through the fluid flow path, the contents of the second syringe and the first syringe to form a pharmaceutical composition; and administering the pharmaceutical composition to the subject via subcutaneous injection through the second syringe after disconnecting the second syringe from the first syringe.

Aspect 66: The method of Aspect 65, wherein positioning the displaceable member in the second position comprises moving the displaceable member from the first position to the second position.

Aspect 67: The method of Aspect 65 or 66, wherein mixing first comprises moving the contents of the second syringe into the first syringe.

Aspect 68: The method of any one of Aspects 65-67, wherein mixing, through the fluid flow path, comprises cyclically mixing contents between the first and second syringes through a number of mixing cycles.

Aspect 69: The method of any one of Aspects 65-68, comprising mixing the contents of the first and second syringe for 30 or fewer mixing cycles.

Aspect 70: The method of any one of Aspects 65-68, comprising mixing the contents of the first and second syringe for 25 or fewer mixing cycles.

Aspect 71: The method of any one of Aspects 65-68, comprising mixing the contents of the first and second syringe for 20 or fewer mixing cycles.

Aspect 72: The method of any one of Aspects 65-71, wherein the first syringe is formulated to deliver 100 mg to 170 mg of N-methyl-2-pyrrolidone and 75 mg to 90 mg of a 50:50 poly(lactic acid-co-glycolic acid) (PLGA) copolymer; and the second syringe is formulated to deliver 7.5 mg leuprolide acetate and 5 mg to 40 mg of N-methyl-2-pyrrolidone.

Aspect 73: The method of Aspect 72, further comprising repeating the positioning, mixing, and administering steps once per month.

Aspect 74: The method of any one of Aspects 65-71, wherein the first syringe is formulated to deliver 90 mg to 170 mg of N-methyl-2-pyrrolidone and 150 mg to 170 mg of a 75:25 poly(lactide-co-glycolide) (PLG) copolymer; and the second syringe is formulated to deliver 22.5 mg leuprolide acetate and 25 mg to 105 mg of N-methyl-2-pyrrolidone.

Aspect 75: The method of Aspect 74, further comprising repeating the positioning, mixing, and administering steps once every three months.

Aspect 76: The method of any one of Aspects 65-71, wherein the first syringe is formulated to deliver 80 mg to 200 mg of N-methyl-2-pyrrolidone and 145 mg to 185 mg of a 75:25 poly(lactide-co-glycolide) (PLG) copolymer; and the second syringe is formulated to deliver 30 mg leuprolide acetate and 55 mg to 120 mg of N-methyl-2-pyrrolidone or 35 mg to 135 mg N-methyl-2-pyrrolidone.

Aspect 77: The method of Aspect 76, further comprising repeating the positioning, mixing, and administering steps once every three months.

Aspect 78: The method of any one of Aspects 65-71, wherein the first syringe is formulated to deliver 80 mg to 225 mg of N-methyl-2-pyrrolidone and 190 mg to 230 mg of a 75:25 poly(lactide-co-glycolide) (PLG) copolymer; and the second syringe is formulated to delivery 30 mg leuprolide acetate and 30 mg to 135 mg of N-methyl-2-pyrrolidone. Aspect 79: The method of Aspect 78, further comprising repeating the positioning, mixing, and administering steps once every four months.

Aspect 80: The method of any one of Aspects 65-71, wherein the first syringe is formulated to deliver 100 mg to 170 mg of N-methyl-2-pyrrolidone and 145 mg to 185 mg of a 85:15 poly(lactide-co-glycolide) (PLG) copolymer; and the second syringe is formulated to deliver 45 mg leuprolide acetate and 55 mg to 95 mg of N-methyl-2-pyrrolidone.

Aspect 81: The method of any one of Aspects 65-80, wherein the subject has prostate cancer, is a pediatric patient 2 years of age or older having central precocious puberty (CPP), or has hormone receptor-positive breast cancer.

### Examples

The examples further illustrate this disclosure. The scope of the disclosure and claims is not limited by the scope of the examples. The following examples describe methods used to prepare and test the extended-release compositions disclosed herein.

### I. Preparation of Polymers

Polymers were either purchased (RESOMER® polymers) or synthesized in-house. Poly(D.L-lactide-co-glycolide) (PLG) copolymers or poly-DL-lactide (PLA) polymers were produced using the following methods. The amounts of monomers (DL-lactide or glycolide) and initiator (e.g., glycolic acid, 1,6-Hexanediol) were selected to obtain a targeted initiator, monomer molar ratio, and weight average molecular weight for each investigated polymer. The monomer molar ratios and weight average molecular weights reported in individual examples are targeted values unless specified as actual or experimental. The polymerization was conducted in a stirred, heated vessel under a nitrogen atmosphere. In the vessel, appropriate amounts of monomers (DL-lactide and/or glycolide), and initiator (glycolic acid or 1,6-Hexanediol) were added, the vessel contents were placed under a nitrogen atmosphere. The temperature of the vessel was increased until the reagents melted. A catalyst solution was made with appropriate amounts of stannous octoate and toluene and added to the vessel. The vessel was then heated to about 140-170° C. under a nitrogen atmosphere for about 4-18 hours (depending on the polymer of interest) with constant stirring. Then, the vessel was evacuated to remove unreacted monomers, and the monomers were vacuum-distilled out of the polymerization mixture. The hot melt was then extruded into cooling pans. After cooling, the solid mass was broken up into smaller pieces. The polymer was purified as needed using the solvent/non-solvent-induced phase separation method.

### II. Preparation of Bulk Polymer Solutions and Prefilled Syringes (First Container-Syringe A) of Polymer Solution

Polymer bulk solutions were prepared by weighing a known amount of each polymer material into individual Flack-Tek jars of the desired size. A known amount of NMP was added to each polymer, and the containers were placed on a horizontal jar mill or Turbula for mixing at room temperature. Jars were mixed until a visually clear homogenous polymer solution is obtained indicating the complete dissolution of the polymer in the solvent. Prefilled syringes (First container-Syringe A, referred to elsewhere as “first syringe”) containing polymer delivery system were prepared by weighing the required amounts of polymer solution into 1.2 mL female polypropylene syringe barrel with Luer lock and plunger tip and capped with male polypropylene syringe cap, 1.2 mL male cyclic olefin copolymer syringes, or 1 mL male cyclic olefin copolymer syringes with female polypropylene cap. In some instances, the two syringes were coupled via the connector prior to packaging. Filled syringes were then packaged in labeled foil pouches or a plastic tray pack with foil lid with a Syringe B (referred to elsewhere as the “second syringe,” or the syringe containing the active pharmaceutical ingredient) and a desiccant pack and the pouches were sealed. After filling the syringes with the formulations, they were terminally sterilized with an external radiation dose of 30 kGy e-beam processing. Terminally sterilized syringes kits were stored under refrigerated conditions (e.g., 2-8° C.) or accelerated conditions (>25° C. see individual experiments) in the sealed foil pouches.

### III. Preparation of LA-DS Organic-Solvent Bulk Solution and Prefilled Syringes (Second Container-Syringe B)

To produce the drug/solvent bulk solution comprising the API (LA), the desired amount of the LA was combined with the solvent N-methyl-2-pyrrolidone (NMP), in the indicated amounts (see individual experiments below). The API and solvent were combined in a glass vial or jar. Jars were mixed using the jar mill, Turbula, or shaker at room temperature until homogeneous. LA is soluble in NMP and can load higher concentrations (at least 40% w/w). After dissolving the API in the organic solvent, the bulk solution was manually filled into syringes and capped with a tip cap. The syringes used for filling were either 1.2 mL male polypropylene syringes, 1.2 mL male cyclic olefin copolymer syringes or 1 mL male cyclic olefin copolymer syringes. In some instances, the two syringes were coupled via the connector prior to packaging. Filled syringes were then packaged in labeled foil pouches or a plastic tray pack with foil lid with a Syringe A (polymer-solvent solution) and a desiccant pack and the pouches were sealed. After filling the syringes with the formulations, they were terminally sterilized with an external radiation dose of more than 15 kGy and less than 60 kGy from irradiation processing (E-beam, Gamma or X-ray). After terminal sterilization, the syringes were stored under refrigerated conditions (e.g., 2-8° C.) or accelerated conditions (>25° C. see individual experiments).

IV. Preparation of Final Formulation for administration:

Immediately before administering, “A” and “B” syringes were allowed to equilibrate to room temperature for not less than 30 minutes. The syringes were coupled and mixed by cycling the contents from one syringe to the other to visual and/or substantial homogeneity. The mixed formulation was fully transferred to the male dosing syringe for delivery and testing. A safety needle of 18 or 20 g was used for delivering the formulation when needed during testing. As will be understood by one skilled in the art, the “A” syringe refers to the “first syringe,” or the syringe containing the polymer and solvent, whereas the “B” syringe refers to the “second syringe,” or the syringe that includes the leuprolide acetate either in lyophilized or solubilized form, e.g., solubilized in NMP.

### V. HPLC Analysis of the Formulations

LA/organic solvent solutions were prepared for HPLC analysis by dilution to volume with mobile phase A (0.1% trifluoroacetic acid water) and mixing thoroughly by vortex. Dilutions were performed as needed using volumetric glassware and mixed thoroughly by vortex mixing. A second dilution of 2 mL to 20 mL was performed with mobile phase A to obtain a working sample.

For the HPLC test sample preparation of formulations that contain polymer, syringe A and syringe B were coupled together and mixed for 45 cycles. The product was dispensed from the final mixed syringe formulation content into a 50 mL volumetric flask and the weight was recorded. 2.0 mL of mobile phase B (0.1% trifluoroacetic acid acetonitrile) was added and mixed by swirling. 3.0 mL of mobile phase A was added and mixed by swirling. Samples were placed in a water bath at 55° C.±2° C. for 30 minutes and then cooled to room temperature. Samples were diluted to volume with mobile phase A and mixed thoroughly by vortex, followed by dilution to 20 mL with mobile phase A with thorough mixing. A second dilution of 2 mL to 20 mL was performed with mobile phase A to obtain a working sample. The sample solutions were filtered through a 0.45 μm PTFE syringe filter into amber HPLC vials before collection into the HPLC vial.

Additionally, samples of the liquid-liquid drug product were prepared by mixing for the indicated number of cycles and dispensed into an organic solution comprising acetonitrile, methanol, and trifluoroacetic acid. The drug product was dissolved via vortex as before and then diluted to 100 mL with a predominantly aqueous diluent of water, acetonitrile, and trifluoroacetic acid. Filtration and subsequent dilutions of the stock to an approximate concentration of 30 to 40 μg/mL of leuprolide were performed to generate working samples.

HPLC analysis for assay and related compounds was performed using an Agilent AdvanceBio Peptide 3.0×100 mm, 2.7 um column and an Agilent AdvanceBio Peptide Map Guard 3.0×5 mm 2.7 um guard column at 30° C. with a flow rate of 0.75 mL/min. The runtime was 15 minutes with a 10-μL injection for assay and related compounds for the solution gel depot formulation, and a 20-μL injection for assay and related compounds for the polymer depot formulation. Detection was performed with a diode array detector set at 220 nm.

Additionally, HPLC analysis was also performed using a Waters Cortecs UPLC column and matching guard column at 40° C. with a 0.5 mL/min flow rate. A gradient method utilizing both acidic water and acetonitrile was employed to sufficiently separate N-methyl pyrrolidone and leuprolide. Detection was performed with a diode array detector set to 220 nm.

### VI. In-Vitro Release Testing (IVRT) and F2 Similarity Calculations

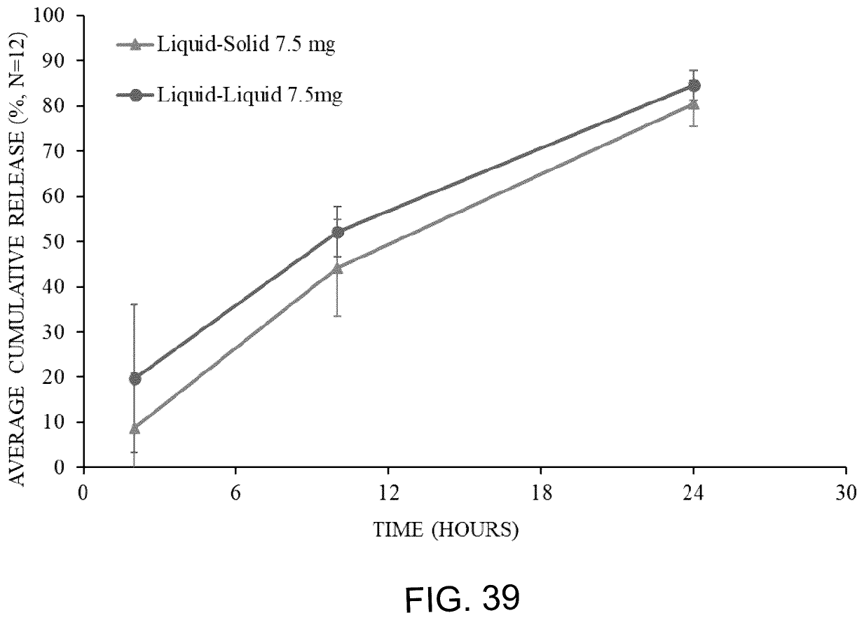

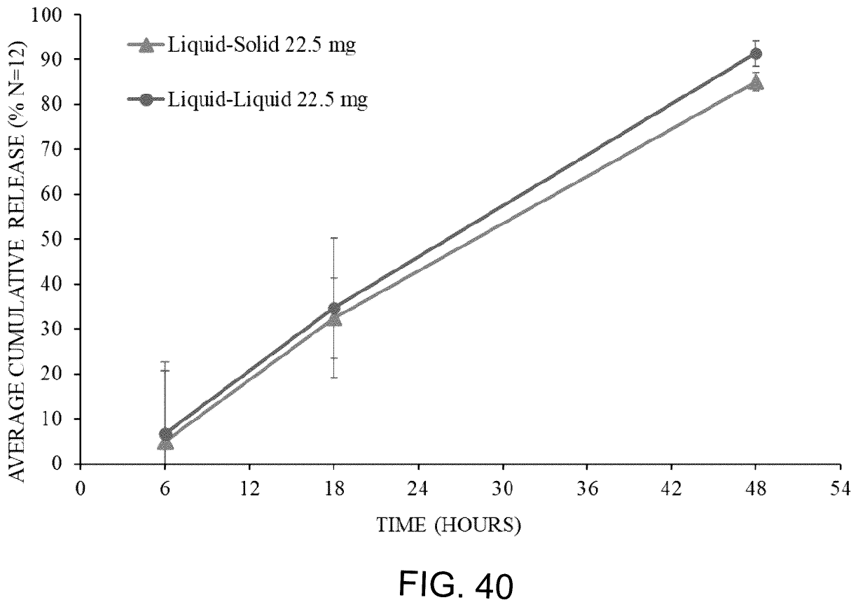

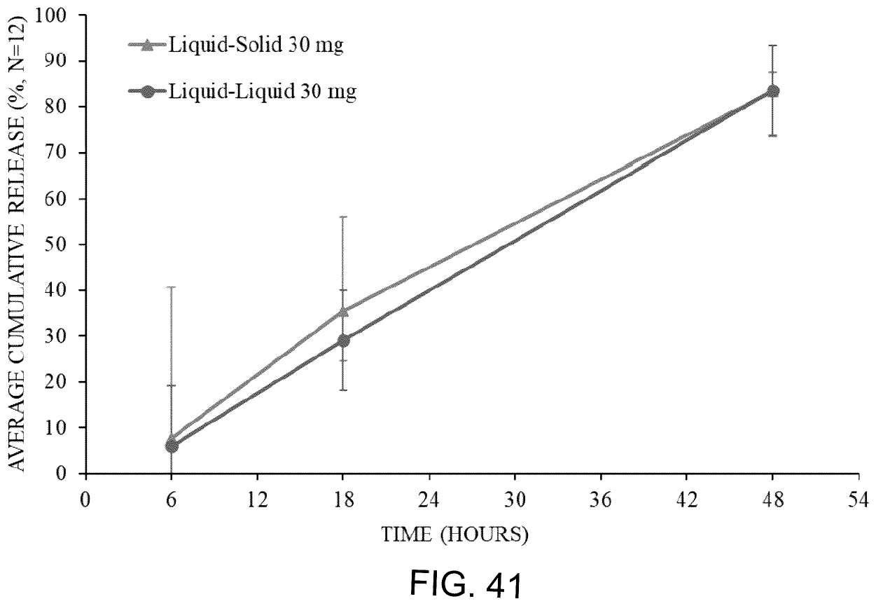

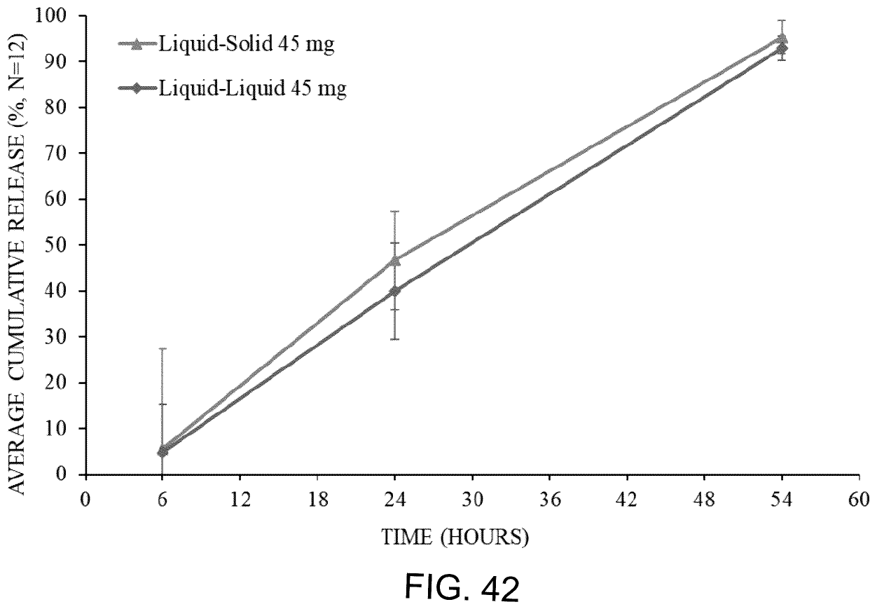

Dissolution media was prepared for in vitro release analysis of formulations. A precise volume of media was measured into a glass jar and conditioned to 60° C. in a temperature-controlled reciprocating water bath prior to formulation reconstitution. Room temperature samples were mixed per package instructions and transferred into Syringe B for addition to release media. An 18- or 20-gauge needle was attached to Syringe B for controlled expression of the homogeneous formulation into a 60° C. media jar using a syringe pump. Jars were removed from the reciprocating bath and 2 mL of media was sampled just below the surface of the media at pre-determined time points for each formulation. These media samples were then analyzed for LA content by HPLC similar to the above HPLC assay description. The in vitro release data is summarized in FIG. 39, FIG. 40, FIG. 41, and FIG. 42. F2 similarity calculations were performed to compare liquid-liquid with liquid-solid formulation in vitro performance for each dosage strength. These similarity calculations showed there are no statistically significant differences between liquid-liquid and liquid-solid formulation final delivered product release performance. F2 similarity calculations measure the closeness between two profiles and were performed:

$$F 2 = 50 \times log [ {( 1 + \frac{1}{n} {\sum }_{t = 1}^{n} {( {R}_{t} - {T}_{t} )}^{2} )}^{- 0.5} \times 100 ]$$

Where n is the number of time points, Rt is the dissolution value of the reference at time t, and Tt is the dissolution value of the test sample at time t. F2 values above 50 are considered similar. These similarity calculations showed there are no statistically significant differences between liquid-liquid and liquid-solid formulations, providing evidence that the liquid-liquid formulation performs the same as the liquid-solid formulation under these IVRT conditions.

### VII. Animal Dosing

The first container comprising the polymer-solvent solutions (Syringe A) and the second container comprising the LA solvent solutions (Syringe B) were prepared as described above. For animal study dose administration, the LA content was measured by HPLC assay. This % w/w LA in the homogeneous formulation was used to calculate dosing volume for animal subjects at a predetermined amount of LA per kilogram of body weight. The calculated volume for each formulation was transferred to a graduated 100 microliter Hamilton syringe and injected through an 18- or 20-gauge needle into the subjects. LA absorption rates were obtained using a rat model. Male rats were each injected with a single subcutaneous injection of the extended-release compositions/formulations. The composition of the formulations tested in this experiment are listed in Tables 1, 2 and 4. At predetermined time points, rats were bled and plasma LA levels were determined using liquid chromatography with tandem mass spectrometry (LC-MS/MS). Each data point is based on an average plasma or average serum LA concentration. Six rats were dosed for each group, with sparse blood sampling performed for early time points. A drug product that is a liquid-solid formulation (containing the same polymer-solvent but in the amounts indicated in Syringe A and lyophilized LA in Syringe B as indicated in the table) was included in each study as a control.

TABLE 1
Comparison of Contents in Syringe A/B for Liquid-Solid Formulations
vs Container A/B for Liquid-Liquid Formulations - 7.5 mg

| | | Liquid-Solid | | Liquid-Liquid | |
|---|---|---|---|---|---|
| | | Formulation | | Formulation | |
| | | | Delivered | | Delivered |
| | | | Mass | | Mass |
| Component | Excipient | % w/w | (mg) | % w/w | (mg) |
| Delivery | 50:50 Poly(DL- | 33.0 | 82.5 | 33.0 | 82.5 |
| System | lactide-co- | | | | |
| (Syringe A) | glycolide) | | | | |
| | N-methyl-2- | 64.0 | 160.0 | 58.4 | 146.0 |
| | pyrrolidone | | | | |
| Drug | Leuprolide | 3.0 | 7.5 | 3.0 | 7.5 |
| Substance | Acetate | | | | |
| (Syringe B) | N-methyl-2- | N/A | N/A | 5.6 | 14.0 |
| | pyrrolidone | | | | |

| | | |
|---|---|---|
| Final Delivered Product | 250 mg | 252 mg |

TABLE 2
Comparison of Contents in Syringe A/B for Liquid-Solid Formulations
vs Container A/B for Liquid-Liquid Formulations - 22.5 mg

| | | Liquid-Solid | | Liquid-Liquid | |
|---|---|---|---|---|---|
| | | | Delivered | | Delivered |
| | | | Mass | | Mass |
| Component | Excipient | % w/w | (mg) | % w/w | (mg) |
| Delivery | 75:25 Poly(DL- | 42.3 | 158.6 | 42.3 | 158.6 |
| System | lactide-co- | | | | |
| (Syringe A) | glycolide) | | | | |
| | N-methyl-2- | 51.7 | 193.9 | 40.3 | 151.1 |
| | pyrrolidone | | | | |
| Drug | Leuprolide | 6.0 | 22.5 | 6.0 | 22.5 |
| Substance | Acetate | | | | |
| (Syringe B) | N-methyl-2- | N/A | N/A | 11.4 | 42.8 |
| | pyrrolidone | | | | |

| | | |
|---|---|---|
| Final Delivered Product | 375 mg | 375 mg |

TABLE 3
Comparison of Contents in Syringe A/B for Liquid-Solid Formulations
vs Container A/B for Liquid-Liquid Formulations - 30 mg

| | | Liquid-Solid | | Liquid-Liquid | |
|---|---|---|---|---|---|
| | | % | Delivered | % | Delivered |
| Component | Excipient | w/w | Mass (mg) | w/w | Mass (mg) |
| Delivery | 75:25 Poly(DL- | 42.3 | 211.5 | 42.3 | 211.5 |
| System | lactide-co-glycolide) | | | | |
| (Syringe A) | N-methyl-2- | 51.7 | 258.5 | 40.3 | 201.5 |
| | pyrrolidone | | | | |
| Drug | Leuprolide Acetate | 6.0 | 30.0 | 6.0 | 30.0 |
| Substance | N-methyl-2- | N/A | N/A | 11.4 | 57.0 |
| (Syringe B) | pyrrolidone | | | | |

| | | |
|---|---|---|
| Final Delivered Product | 500 mg | 500 mg |

TABLE 4
Comparison of Contents in Syringe A/B for Liquid-Solid Formulations
vs Container A/B for Liquid-Liquid Formulations - 45 mg

| | | Liquid-Solid | | Liquid-Liquid | |
|---|---|---|---|---|---|
| | | % | Delivered | % | Delivered |
| Component | Excipient | w/w | Mass (mg) | w/w | Mass (mg) |
| Delivery | 85:15 Poly(DL- | 44.0 | 165.0 | 38.1 | 165.0 |
| System | lactide-co-glycolide) | | | | |
| (Syringe A) | N-methyl-2- | 44.0 | 165.0 | 35.5 | 153.6¹ |
| | pyrrolidone | | | | |
| Drug | Leuprolide Acetate | 12.0 | 45.0 | 10.4 | 45.0 |
| Substance | N-methyl-2- | N/A | N/A | 16.0 | 69.6¹ |
| (Syringe B) | pyrrolidone | | | | |

| | | |
|---|---|---|
| Final Delivered Product | 375.0 mg | 433.2 mg |

¹Total NMP delivered from the Liquid-Liquid product is 223.2 mg, 58.2 mg higher than the NMP in the Liquid-Solid 45 mg formulation. This corresponds to a 35.3% difference in delivered NMP and a 15.5% difference in final product delivered mass.

TABLE 5
Comparison of Contents in Syringe A/B for Liquid-Solid Formulations
vs Container A/B for Liquid-Liquid Formulations - 30 mg

| | | Liquid-Solid | | Liquid-Liquid | |
|---|---|---|---|---|---|
| | | % | Delivered | % | Delivered |
| Component | Excipient | w/w | Mass (mg) | w/w | Mass (mg) |
| Delivery | 75:25 Poly(DL- | 41.7 | 166 | 40.4 | 165.6 |
| System | lactide-co-glycolide) | | | | |
| (Syringe A) | N-methyl-2- | 50.8 | 202 | 38.4 | 157.4¹ |
| | pyrrolidone | | | | |
| Drug | Leuprolide Acetate | 7.5 | 30 | 7.3 | 30 |
| Substance | N-methyl-2- | N/A | N/A | 13.9 | 57.0¹ |
| (Syringe B) | pyrrolidone | | | | |

| | | |
|---|---|---|
| Final Delivered Product | 398 mg | 410 mg |

¹Total NMP delivered from the Liquid-Liquid product is 213 mg, 11 mg higher than the Liquid-Solid formulation. This corresponds to a 5.32% difference in delivered NMP and a 2.76% difference in final product delivered mass.

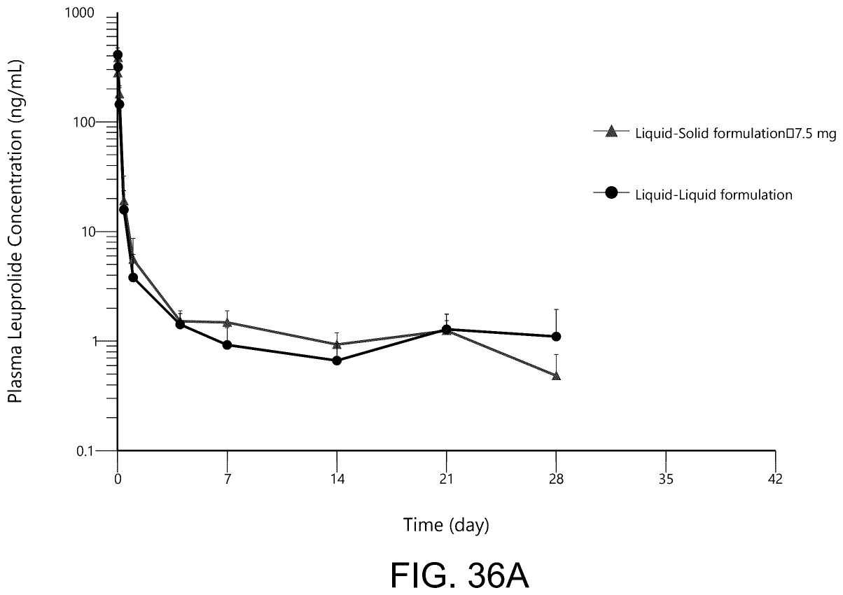

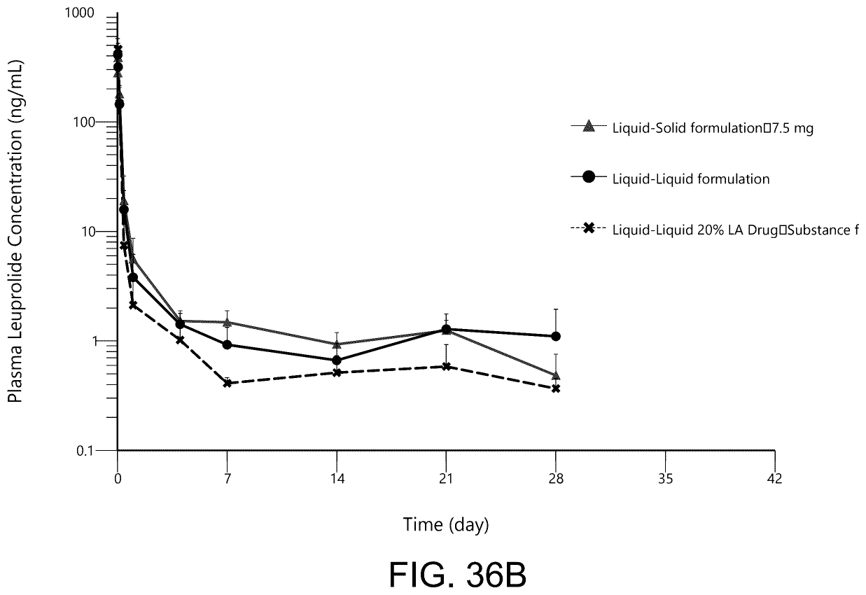

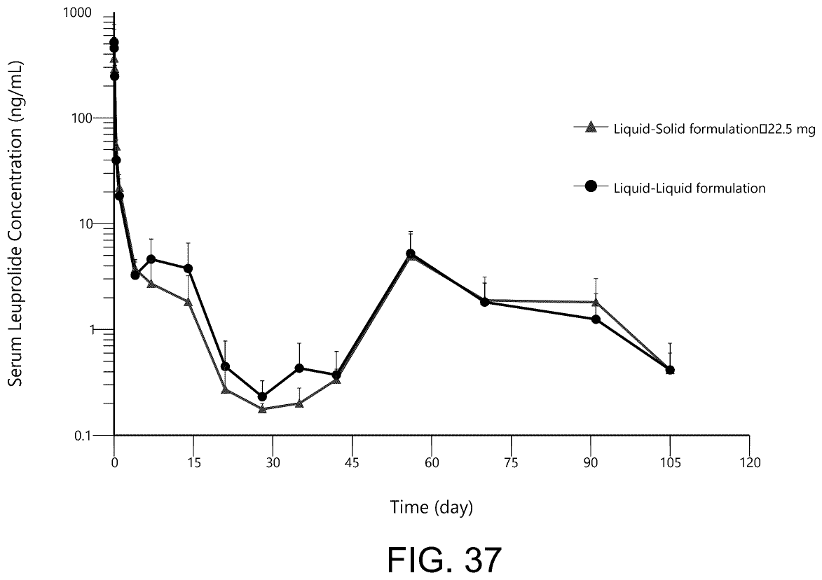

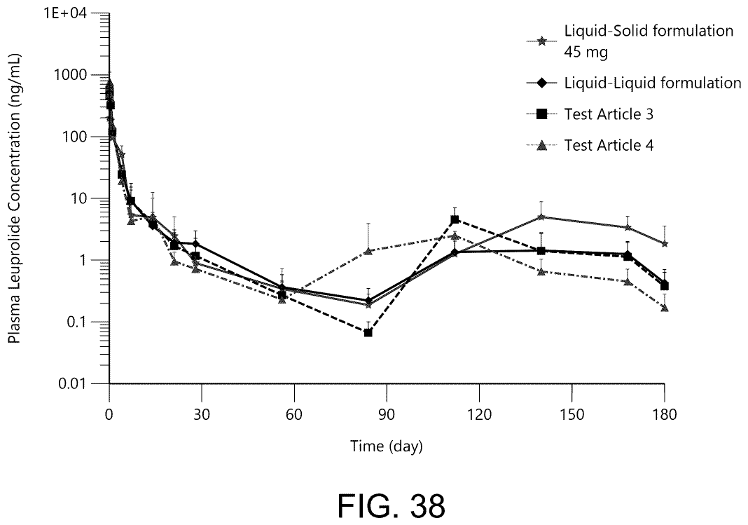

Blood plasma or some instances serum concentrations of LA are presented for each study in FIG. 36A, FIG. 37, and FIG. 38. Cₘₐₓ and AUCₗₐₛₜ were similar for the Liquid-Liquid formulations when compared to the corresponding Liquid-Solid formulations delivering 7.5 mg, 22.5 mg, and 45 mg as controls.

### VIII. 7.5 mg Product:

The LA plasma concentration data of the Liquid-Liquid 7.5 mg product demonstrates similar controlled release exposure of this combination product to the liquid-solid 7.5 mg control (FIG. 36A). This is specifically observed in the results after day 28; where LA release is complete (day 35 and 45 concentrations were BLQ). No significant difference was observed for Liquid-Solid 7.5 mg formulation and the Liquid-Liquid 7.5 mg formulation mean±SEM AUC_{last, 0-28 d} (106.09±7.94 and 91.66±11.56, respectively; p-value 0.3209, n=8). In addition, no significant difference was observed for Liquid-Solid 7.5 mg formulation and the Liquid-Liquid 7.5 mg formulation mean Cₘₐₓ±SEM (384.25±20.22 and 410±31.56, respectively; p-value 0.5033, n=8). Similarly, the Liquid-Liquid 20% LA formulation was comparable to both the Liquid-Solid and Liquid-Liquid 7.5 mg formulations.

TABLE 6
PK Parameters for 7.5 mg and 20% LA Formulations

| | | | Liquid-Liquid |
|---|---|---|---|
| | | | 20% LA Drug |
| | Liquid-Solid | Liquid-Liquid | Substance |
| Group | formulation 7.5 mg | formulation | Formulation |
| Cmax (ng/mL) | 384.25 ± 20.22 | 410.00 ± 31.56 | 460.00 ± 30.08 |
| (mean +/− SE) | | | |
| P value | — | 0.5033 | 0.0581 |
| AUC0-28 d | 106.09 ± 7.94 | 91.66 ± 11.56 | 81.72 ± 4.28 |
| (day*ng/mL) | | | |
| (mean +/− SE) | | | |
| P value | — | 0.3209 | 0.02097 |

### IX. 22.5 mg Product:

As shown in FIG. 37, no significant difference was observed for Liquid-Solid 22.5 mg formulation and the Liquid-Liquid 22.5 mg product mean±SEM AUC_{last, 0-105 d} (335.74±35.25 and 332.23±35.25, respectively; p-value 0.9368, n=6). In addition, no significant difference was observed for Liquid-Solid 22.5 mg formulation and the Liquid-Liquid 22.5 mg formulation mean Cₘₐₓ±SEM (544.33±79.51 and 519±139.20, respectively; p-value 0.8776, n=6). Serum LA concentrations were BLQ after study day 105.

### X. 45 mg Product:

In addition to the Liquid-Liquid 45 mg product, the impact of increased final delivered product NMP content on PK was investigated. Table 6 details the composition of each test formulation delivering the same amount of LA and polymer as Liquid-Solid 45 mg formulation while varying the amount of delivered NMP. The range of NMP content studied was designed to span from delivered amounts of Liquid-Solid 45 mg formulation (165 mg NMP) to Liquid-Solid 30 mg formulation (258.5 mg NMP) in the 45 mg Liquid-Liquid formulations. The results through day 180 show similar PK profiles for the control and all test articles.

TABLE 7
45 mg Formulations Studied in the Animal Model

| | Product | | | |
|---|---|---|---|---|
| | Liquid-Solid | | | |
| | Formulation | Liquid-Liquid Formulation Device | | |
| | Excipient | | | |
| | Liquid-Solid | Liquid-Liquid | Test | Test |
| | Control | Formulation | Article 3 | Article 4 |
| Delivery System | 50% PLG | 55% PLG | 52.5% PLG | 50% PLG |
| (Syringe A / Tip Chamber) | 50% NMP | 45% NMP | 47.5% NMP | 50% NMP |
| Drug Substance | 100% LA | 35% LA / 65% NMP | | |
| (Syringe B / Flange Chamber) | | | | |
| Final Delivered Product NMP % | 44.0% | 51.5% | 52.9% | 54.5% |
| Target Delivered Mass | 375 mg | 445 mg | 460 mg | 475 mg |
| Theoretical Delivered NMP | 165 mg | 229 mg | 244 mg | 259 mg |

TABLE 8
Pharmacokinetic Parameters of LA following a single subcutaneous
injection of Liquid-Solid 45 mg and Liquid-Liquid formulations
with various NMP concentrations

| | Liquid- | | | |
|---|---|---|---|---|
| | Solid | Liquid- | Test | Test |
| | 45 mg | Liquid | Article 3 | Article 4 |
| | (mean ± | (mean ± | (mean ± | (mean ± |
| | SEM, | SEM, | SEM, | SEM, |
| Group | n = 6) | n = 6) | n = 6) | n = 6) |
| Cₘₐₓ (ng/mL) | 532.33 ± | 550.67 ± | 572.00 ± | 763.33 ± |
| | 41.90 | 39.75 | 115.05 | 193.78 |
| p value compared to | — | 0.7575 | 0.7526 | 0.271 |
| Liquid-Solid 45 mg | | | | |
| AUC_{last, 0-180 d} | 885.35 ± | 834.57 ± | 873.93 ± | 918.14 ± |
| (day*ng/mL) | 116.75 | 105.83 | 42.23 | 59.72 |
| p value compared to | — | 0.754 | 0.9296 | 0.8093 |
| Liquid-Solid 45 mg | | | | |

The non-GLP in vivo studies show that the Liquid-Liquid formulations are similar in release to that of the Liquid-Solid 7.5 mg, 30 mg, and 45 mg formulations. The Liquid-Liquid 45 mg formulation, which contains an additional 58 mg of NMP, does not alter LA exposure. NMP is a biocompatible organic solvent currently used in various products up to 833 mg per unit dose. NMP is also listed in the Inactive Ingredient Database for subcutaneous administration at 376 mg per unit dose.

In general, it is established that concentration of drug, polymer, and solvent in in situ forming depots is an important factor in controlling depot formation and associated drug release. See, e.g., Gomaa, E.; Eissa, N. G.; Ibrahim, T. M.; El-Bassossy, H. M.; El-Nahas, H. M.; Ayoub, M. M. “Development of depot PLGA-based in-situ implant of Linagliptin: Sustained release and glycemic control.” Saudi Pharm J 2023, 31 (4), 499-509; Parent, M.; Nouvel, C.; Koerber, M.; Sapin, A.; Maincent, P.; Boudier, A. “PLGA in situ implants formed by phase inversion: critical physicochemical parameters to modulate drug release.” J Control Release 2013, 172 (1), 292-304. The window in which solvent concentration may be tuned without substantially affecting the release rate depends on the identity of the polymer, the drug, and solvent. Therefore, to minimize the risk of changes in in vivo release profile, it the mixed product composition was maintained as close to the liquid/solid formulation as possible.

### XI. LA Gelation Studies

Drug solutions were prepared as described above and filled into syringes or glass vials. Syringes were untreated, contained various amounts of silicone, or were plasma coated for lubrication. Samples were irradiated by e-beam irradiation at not less than 25 kGy and stored at accelerated conditions of 60° C. with agitation at 150 rpm for up to 16 days. Samples were inspected periodically to determine if the drug solution remained a solution or had formed a solid/gel by an inversion test. Samples that had “gelled” were transferred to 5° C. for subsequent testing.

The gelation rate for each sample was calculated as Rate=1/(simulated 5° C. age at time of gelation in days). The Arrhenius equation was used to determine the simulated age, assuming standard reaction rate doubling for every 10° C. increase in temperature. Samples that did not gel were given a gelation rate of 0. Data was analyzed using JMP. Variables related to peptide concentration in the solution (drug solution concentration, leuprolide content, and assay) and their interactions significantly contributed to gelation rate (p<0.05). Container type and lubrication were not significant.

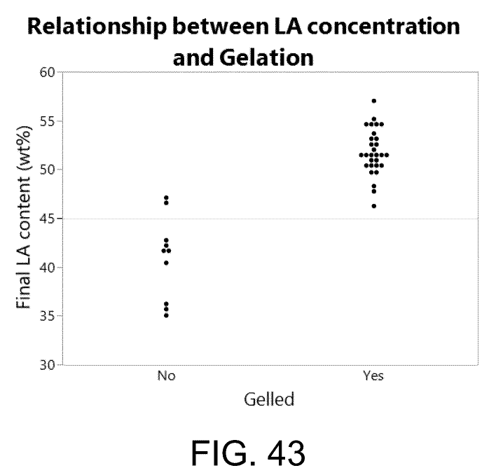

Samples were tested for leuprolide content by HPLC. Leuprolide content was then converted to leuprolide acetate by multiplying by 1.078 (based on molecular mass ratios). The results are shown in FIG. 43. A marked difference in leuprolide acetate content between the gelled and non-gelled samples was seen, with the tipping point between solution and solid/gelled samples occurring at about 45% LA.

### XII. Comparative Example Demonstrating Instability of Pre-Mixed System

55.5 wt % polymer solution was prepared by combining 85:15 PLG polymer and NMP in a container and mixing on a Turbula until homogeneous. 39.96 wt % LA drug solution was similarly prepared by combining LA and NMP in a container and mixing on a Turbula until homogeneous. Drug and polymer solutions were then filled into separate syringes, the syringes coupled, and the two solutions were mixed. After mixing, samples were stored at 37° C. Samples were periodically tested for polymer Mw and LA content. The simulated 5° C. age was calculated using the Arrhenius equation assuming the reaction rate doubles for every 10° C. increase in temperature. A decrease in both polymer Mw and LA assay was detected over time, indicating the polymer and drug are not stable long-term when mixed.

TABLE 9
Reduction of Polymer M_{w} and LA Assay in Pre-Mixed System

| | Approx. | | | |
|---|---|---|---|---|
| 37° C. Age | 5° C. age | Polymer | | LA Assay |
| (weeks) | (weeks) | Mw (Da) | PDI | (% w/w) |
| 0 | 0 | 22,097 | 1.54 | 10.174 |
| 1 | 9 | 16,822 | 1.55 | 7.418 |
| 2 | 18 | 13,706 | 1.59 | 7.674 |

### XIII. Examples Demonstrating Importance of Polymer Solution Viscosity

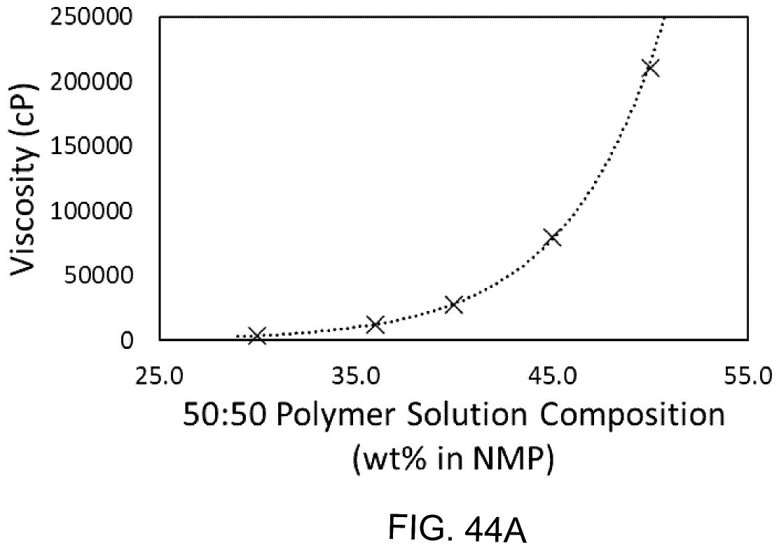

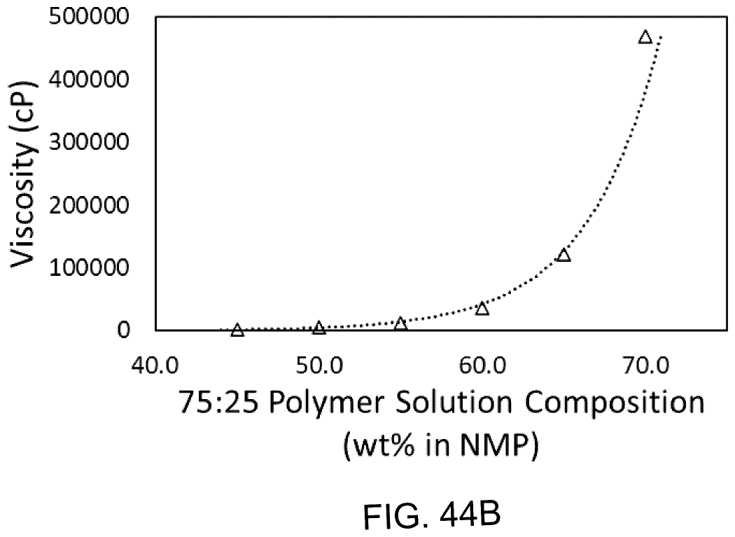

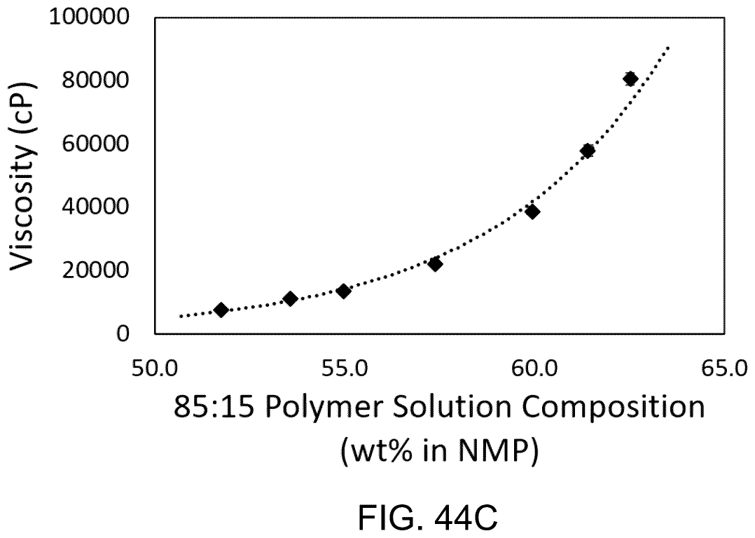

Polymer solutions were prepared by combining polymer and NMP in a container and mixing on a Turbula, rotisserie, or Flaktek mixer with or without gentle heating until homogeneous. Viscosity testing was performed using a Brookfield R/S CPS+ rheometer at 25° C. with a C50-1 cone or a RCT-50-1 cone. Samples were tested with 2-6 replicates and the average result reported. An exponential curve was fit for each polymer, shown in FIGS. 44A-C.

TABLE 10
Solution Composition Viscosity Relationship for PLG Polymers

| | | | | Corresponding |
|---|---|---|---|---|
| Polymer Solution | | | NMP removed | drug solution |
| Polymer | Weight | Viscosity | from polymer | composition |
| ID | % | (cP) | solution (mg) | (wt % LA) |
| 50:50 PLG | 30.0 | 3,687 | — | — |
| (7.5 mg | 34 | — | 0 | 100 |
| strength) | 36.0 | 12,702 | 13 | 36 |
| | 40.0 | 28,037 | 36 | 17 |
| | 45.0 | 80,183 | 59 | 11 |
| | 50.0 | 210,995 | 78 | 9 |
| 75:25 PLG | 45.0 | 1,885 | 0 | 100 |
| (22.5 and | 50.0 | 4,597 | 35 | 39 |
| 30 mg | 55.0 | 11,760 | 64 | 26 |
| strengths) | 60.0 | 35,666 | 88 | 20 |
| | 65.0 | 122,297 | 109 | 17 |
| | 70.0 | 469,010 | 126 | 15 |
| 85:15 PLG | 50 | — | 0 | 100 |
| (45 mg | 51.7 | 7,366 | 11 | 81 |
| strengths) | 53.6 | 11,204 | 22 | 67 |
| | 55.0 | 13,374 | 30 | 60 |
| | 57.4 | 21,673 | 43 | 51 |
| | 59.9 | 39,029 | 55 | 45 |
| | 61.4 | 57,879 | 61 | 42 |
| | 62.5 | 80,514 | 66 | 41 |

The results show an exponential relationship, with solutions becoming increasingly viscous as polymer concentration increases. An exponential curve was fit to each dataset. For the 50:50 PLG solutions, Viscosity (cP)=8.4757*e^{0.2028*polymer solution %}. For the 75:25 PLG solutions, Viscosity (cP)=0.0771*e^{0.2202*polymer solution %}. For the 85:15 PLG solutions, Viscosity (cP)=0.0945*e^{0.2169*polymer solution %}. High viscosities, e.g., those above about 20,000 cP, can present challenges for filling syringes with respect to necessary throughput, accuracy, and precision. Additionally, mixing the viscous solution by the user can be difficult due to the high forces required. Therefore, it can be advantageous to remove only a certain amount of NMP from the polymer solution and add this NMP to the drug solution to dissolve the LA. This creates an effective lower limit on the wt % of the drug solution in some embodiments.

For the 7.5 mg formulation (using the 50:50 polymer), keeping polymer solution viscosity below 20,000 cP corresponded to a polymer composition of less than or equal to about 38.3%. Keeping total NMP constant, this relates to a drug composition of 22%. This is below the 45% LA limit where gelation issues were seen (Table 11).

TABLE 11
Illustrative Liquid-Liquid Formulations - 7.5 mg/1 month

| | | | Component | | | | | |
|---|---|---|---|---|---|---|---|---|
| | | | Delivery system | | | | | |
| | | | (Syringe A) | | | Drug | | |
| | | | | | PS | Substance | | |
| | | | 50:50 | | Viscosity | (Syringe B) | | Total |
| | | | PLGH | NMP | (cP)¹ | LA | NMP | (mg) |
| Liquid- Solid | | % w/w | 34.0 | 66.0 | 8370 | 100 | N/A | 250 |
| Formulation | | Delivered | 82.5 | 160.0 | | 7.5 | | |
| | | Mass (mg) | | | | | | |
| Liquid- | 45% LA DS | % w/w | 35.4 | 64.6 | 11,000 | 45 | 55 | 250 |
| Liquid | | Delivered | 82.5 | 151 | | 7.5 | 9 | |
| Formulation | | Mass (mg) | | | | | | |
| | 34% LA DS | % w/w | 36.2 | 63.8 | 13,100 | 34 | 66 | 250 |
| | | Delivered | 82.5 | 145 | | 7.5 | 15 | |
| | | Mass (mg) | | | | | | |
| | 27% LA DS | % w/w | 37.1 | 62.9 | 15,700 | 27 | 73 | 250 |
| | | Delivered | 82.5 | 140 | | 7.5 | 20 | |
| | | Mass (mg) | | | | | | |
| | 24% LA DS | % w/w | 37.7 | 62.3 | 17,700 | 24 | 76 | 250 |
| | | Delivered | 82.5 | 136 | | 7.5 | 24 | |
| | | Mass (mg) | | | | | | |
| | 20% LA DS | % w/w | 38.8 | 61.2 | 22,200 | 20 | 80 | 250 |
| | | Delivered | 82.5 | 130 | | 7.5 | 30 | |
| | | Mass (mg) | | | | | | |
| | 20% LA DS | % w/w | 34.0 | 66.0 | 8370 | 20 | 80 | 280 |
| | (+30 mg NMP) | Delivered | 82.5 | 160.0 | | 7.5 | 30 | |
| | | Mass (mg) | | | | | | |
| | 24% LA DS | % w/w | 41.5 | 58.5 | 38,300 | 24 | 76 | 230 |
| | (−20 mg NMP) | Delivered | 82.5 | 115 | | 7.5 | 24 | |
| | | Mass (mg) | | | | | | |

¹Illustrative formulations assume 100% pure drug substance (LA). Actual compositions may vary slightly based on purity and significant figures. Per prediction equation: Viscosity (cP) = 8.4757 * e^{0.2028 * polymer solution %} and rounded to 3 significant figures.

For the 22.5 and 30 mg formulations (using the 75:25 PLG), keeping polymer solution viscosity below 20,000 cP corresponded to a polymer composition of less than or equal to about 56.6%. Keeping total NMP constant, this relates to a drug composition of 24%. This is below the 45% LA limit where gelation issues were seen (Tables 12-14).

TABLE 12
Illustrative Liquid-Liquid Formulations - 22.5 mg/3 month

| | | | Component | | | | | |
|---|---|---|---|---|---|---|---|---|
| | | | Delivery system | | | | | |
| | | | (Syringe A) | | | Drug | | |
| | | | | | PS | Substance | | |
| | | | 75:25 | | Viscosity | (Syringe B) | | Total |
| | | | PLG | NMP | (cP)¹ | LA | NMP | (mg) |
| Liquid- Solid | | % w/w | 45 | 55 | 1,550 | 100 | N/A | 375 |
| Formulation | | Delivered | 158.6 | 193.9 | | 22.5 | | |
| | | Mass (mg) | | | | | | |
| Liquid- | 45% LA | % w/w | 48.8 | 51.2 | 3,580 | 45 | 55 | 375 |
| Liquid | DS | Delivered | 158.6 | 166.4 | | 22.5 | 27.5 | |
| Formulation | | Mass (mg) | | | | | | |
| | 34% LA | % w/w | 51.4 | 48.6 | 6,350 | 34 | 66 | 375 |
| | DS | Delivered | 158.6 | 150.0 | | 22.5 | 43.9 | |
| | | Mass (mg) | | | | | | |
| | 27% LA | % w/w | 54.4 | 45.6 | 12,300 | 27 | 73 | 375 |
| | DS | Delivered | 158.6 | 132.9 | | 22.5 | 61.0 | |
| | | Mass (mg) | | | | | | |
| | 24% LA | % w/w | 56.4 | 43.6 | 19,100 | 24 | 76 | 375 |
| | DS | Delivered | 158.6 | 122.6 | | 22.5 | 71.3 | |
| | | Mass (mg) | | | | | | |
| | 20% LA | % w/w | 60.4 | 39.6 | 46,000 | 20 | 80 | 375 |
| | DS | Delivered | 158.6 | 104.0 | | 22.5 | 89.9 | |
| | | Mass (mg) | | | | | | |

¹Illustrative formulations assume 100% pure drug substance (LA). Actual compositions may vary slightly based on purity and significant figures.¹
Per prediction equation: Viscosity (cP) = 0.0771 * e^{0.2202 * polymer solution %} and rounded to 3 significant figures.

TABLE 13
Illustrative Liquid-Liquid Formulations - 30 mg/3 month

| | | | Component¹ | | | | | |
|---|---|---|---|---|---|---|---|---|
| | | | Delivery system | | | | | |
| | | | (Syringe A) | | | Drug | | |
| | | | | | PS | Substance | | |
| | | | 75:25 | | Viscosity | (Syringe B) | | Total |
| | | | PLG | NMP | (cP)² | LA | NMP | (mg) |
| Liquid- Solid | | % w/w | 45 | 55 | 1,550 | 100 | N/A | 398 |
| Formulation | | Delivered | 166 | 202 | | 30 | | |
| | | Mass (mg) | | | | | | |
| Liquid- | 45% LA DS | % w/w | 50.1 | 49.9 | 4,770 | 45 | 55 | 398 |
| Liquid | | Delivered | 166 | 165.3 | | 30 | 36.7 | |
| Formulation | | Mass (mg) | | | | | | |
| | 34% LA DS | % w/w | 53.6 | 46.4 | 10,300 | 34 | 66 | 398 |
| | | Delivered | 166 | 143.7 | | 30 | 58.3 | |
| | | Mass (mg) | | | | | | |
| | 30% LA DS | % w/w | 55.5 | 44.5 | 15,700 | 30 | 70 | 398 |
| | | Delivered | 166 | 133 | | 30 | 69 | |
| | | Mass (mg) | | | | | | |
| | 27% LA DS | % w/w | 57.6 | 42.1 | 24,900 | 27 | 73 | 398 |
| | | Delivered | 166 | 122.0 | | 30 | 79.8 | |
| | | Mass (mg) | | | | | | |
| | 24% LA DS | % w/w | 60.8 | 39.2 | 50,300 | 24 | 76 | 398 |
| | | Delivered | 166 | 107.0 | | 30 | 95.0 | |
| | | Mass (mg) | | | | | | |
| | 20% LA DS | % w/w | 66.9 | 33.1 | 193,000 | 20 | 80 | 398 |
| | | Delivered | 166 | 82.1 | | 30 | 119.9 | |
| | | Mass (mg) | | | | | | |
| | 34% LA DS | % w/w | 51.5 | 48.5 | 6,490 | 34 | 66 | 411 |
| | (+13 mg NMP) | Delivered | 166 | 156.3 | | 30 | 58.7 | |
| | | Mass (mg) | | | | | | |
| | 24% LA DS | % w/w | 56.5 | 43.5 | 19,500 | 24 | 76 | 418 |
| | (+20 mg NMP) | Delivered | 166 | 128 | | 30 | 94 | |
| | | Mass (mg) | | | | | | |
| | 20% LA DS | % w/w | 60.4 | 39.6 | 46,000 | 20 | 80 | 425 |
| | (+27 mg NMP) | Delivered | 166 | 108.8 | | 30 | 120 | |
| | | Mass (mg) | | | | | | |
| | 20% LA DS | % w/w | 56.4 | 43.6 | 19,100 | 20 | 80 | 444 |
| | (+46 mg NMP) | Delivered | 166 | 128.3 | | 30 | 120 | |
| | | Mass (mg) | | | | | | |
| | 34% LA | % w/w | 56.5 | 43.5 | 19,500 | 34 | 66 | 382 |
| | (−16 mg NMP) | Delivered | 166 | 127.8 | | 30 | 58.2 | |
| | | Mass (mg) | | | | | | |

¹Illustrative formulations assume 100% pure drug substance (LA). Actual compositions may vary slightly based on purity and significant figures.
²Per prediction equation: Viscosity (cP) = 0.0771 * e^{0.2202 * polymer solution %} and rounded to 3 significant figures.

TABLE 14
Illustrative Liquid-Liquid Formulations - 30 mg/4 month

| | | | Component¹ | | | | | |
|---|---|---|---|---|---|---|---|---|
| | | | Delivery system | | | | | |
| | | | (Syringe A) | | | Drug | | |
| | | | | | PS | Substance | | |
| | | | 75:25 | | Viscosity | (Syringe B) | | Total |
| | | | PLG | NMP | (cP)² | LA | NMP | (mg) |
| Liquid- Solid | | % w/w | 45 | 55 | 1,550 | 100 | N/A | 500 |
| Formulation | | Delivered | 211.5 | 258.5 | | 30 | | |
| | | Mass (mg) | | | | | | |
| Liquid- | 45% LA | % w/w | 48.8 | 51.2 | 3,580 | 45 | 55 | 500 |
| Liquid | DS | Delivered | 211.5 | 221.9 | | 30 | 36.6 | |
| Formulation | | Mass (mg) | | | | | | |
| | 34% LA | % w/w | 51.4 | 48.6 | 6,350 | 34 | 66 | 500 |
| | DS | Delivered | 211.5 | 200 | | 30 | 58.5 | |
| | | Mass (mg) | | | | | | |
| | 27% LA | % w/w | 54.4 | 45.6 | 12,300 | 27 | 73 | 500 |
| | DS | Delivered | 211.5 | 177.3 | | 30 | 81.2 | |
| | | Mass (mg) | | | | | | |
| | 24% LA | % w/w | 56.5 | 43.5 | 19,500 | 24 | 76 | 500 |
| | DS | Delivered | 211.5 | 162.9 | | 30 | 95.1 | |
| | | Mass (mg) | | | | | | |
| | 20% LA | % w/w | 60.4 | 39.6 | 46,000 | 20 | 80 | 500 |
| | DS | Delivered | 211.5 | 138.7 | | 30 | 119.8 | |
| | | Mass (mg) | | | | | | |

¹Illustrative formulations assume 100% pure drug substance (LA). Actual compositions may vary slightly based on purity and significant figures.
²Per prediction equation: Viscosity (cP) = 0.0771 * e^{0.2202 * polymer solution %} and rounded to 3 significant
figures.

For the 45 mg formulation (using the 85:15 PLG), keeping polymer solution viscosity below 20,000 cP corresponded to a polymer composition of less than or equal to about 56.5%. Keeping total NMP constant, this relates to a drug composition of 54% which is above the limit where gelation occurs. Therefore, it was useful to add additional NMP to the formulation to meet both the polymer and the drug solution composition parameters. To minimize the risk of changes in depot formation and in vivo release behavior, additional NMP was minimized (Table 15).

TABLE 15
Illustrative Liquid-Liquid Formulations - 45 mg/6 months

| | | | Component¹ | | | | | |
|---|---|---|---|---|---|---|---|---|
| | | | Delivery system | | | | | |
| | | | (Syringe A) | | | Drug | | |
| | | | | | PS | Substance | | |
| | | | 85:15 | | Viscosity | (Syringe B) | | Total |
| | | | PLG | NMP | (cP)² | LA | NMP | (mg) |
| Liquid- Solid | | % w/w | 50 | 50 | 4,850 | 100 | NA | 375 |
| Formulation | | Delivered | 165.0 | 165.0 | | 45 | | |
| | | Mass (mg) | | | | | | |
| Liquid- | 45% LA DS | % w/w | 60.0 | 40.0 | 42,400 | 45 | 55 | 375 |
| Liquid | | Delivered | 165.0 | 110 | | 45 | 55 | |
| Formulation | | Mass (mg) | | | | | | |
| | 34% LA DS | % w/w | 58.5 | 41.5 | 30,600 | 34.4 | 65.6 | 413 |
| | (+38 mg NMP) | Delivered | 165.0 | 117.1 | | 45 | 85.9 | |
| | | Mass (mg) | | | | | | |
| | 34% LA DS | % w/w | 56.2 | 43.8 | 18,600 | 34 | 66 | 426 |
| | (+51 mg NMP) | Delivered | 165.0 | 128.6 | | 45 | 87.4 | |
| | | Mass (mg) | | | | | | |
| | 45% LA DS | % w/w | 50 | 50 | 4,850 | 45 | 55 | 430 |
| | (+55 mg NMP) | Delivered | 165.0 | 165.0 | | 45 | 55 | |
| | | Mass (mg) | | | | | | |
| | 34% LA DS | % w/w | 55.4 | 44.6 | 15,600 | 34 | 66 | 430 |
| | (+55 mg NMP) | Delivered | 165.0 | 132.8 | | 45 | 87.2 | |
| | | Mass (mg) | | | | | | |
| | 34% LA DS | % w/w | 55 | 45 | 14,300 | 34.4 | 65.6 | 431 |
| | (+56 mg NMP) | Delivered | 165.0 | 135.0 | | 45 | 86 | |
| | | Mass (mg) | | | | | | |

¹Illustrative formulations assume 100% pure drug substance (LA). Actual compositions may vary slightly based on purity and significant figures.
²Per prediction equation: Viscosity (cP) = 0.0945 * e^{0.2169 * polymer solution %} and rounded to 3 significant figures.

XIV. Improved Mixing and Stability with Liquid-Liquid Formulations

Polymer solutions were prepared by combining polymer and NMP in a container and mixing on a Turbula, rotisserie, or Flaktek mixer with or without gentle heating until homogeneous. Polymer solutions were filled into male (for liquid-liquid formulations) or female (for liquid-solid formulations) syringes. Liquid-liquid formulation drug syringes were prepared by dissolving LA in NMP, gently mixing on a Turbula, rotisserie, or Flaktek mixer until uniform, and then filled into male syringes. Solid drug syringes were prepared by dissolving LA in water, gently mixing until dissolved. Solution was filled into male syringes, and then lyophilized to remove water.

Syringes were coupled using either a male to female (liquid-solid) or male to male (liquid-liquid) couplers with similar fluid paths. They were then packaged in foil pouches or trays and irradiated by e-beam irradiation at not less than 25 kGy. Syringes were mixed for the indicated number of mixing cycles and delivered via an 18 G (45 mg/6 mo) or 20 G (22.5 mg/3 mo) needle. The mixed product was delivered and tested for LA assay, which was expressed as a % of the target weight % in the formulation. The average, standard deviation, and RSD (Stdev/Ave*100%) were then calculated.

Fill weight for these formulations were not finalized in these experiments, as seen by the average assay values not being at 100% of target. However, consistency in the mixed product can be evaluated by comparing the RSD values between groups. Liquid-Solid formulations should be mixed 60 cycles prior to use. This ensures a well-mixed, consistent product. As can be seen, the liquid-liquid formulations have similar or lower RSDs compared to the liquid-solid formulations with substantially fewer mixing cycles.

TABLE 16
22.5 mg / 3 Month Delivered Dose Consistency

| | Liquid - | | | | |
|---|---|---|---|---|---|
| | Solid | | | | |
| | 100% | Liquid-Liquid | | | |
| | LA + | 20.0% LA + | 21.8% LA in | 34.2% LA + | 35.1% LA + |
| | 45% | 60.4% | Drug Syringe + | 51.4% | 52.0% |
| | Polymer | Polymer | 58% Polymer | Polymer | Polymer |
| Mixing Cycles | 60 | 20 | 20 | 20 | 20 |
| N | 6 | 6 | 6 | 10 | 6 |
| Ave (% target) | 119.6 | 113.0 | 95.4 | 108.3 | 103.9 |
| Stdev (% target) | 5.8 | 4.0 | 3.9 | 2.7 | 5.6 |
| RSD (%) | 4.8 | 3.5 | 4.1 | 2.5 | 5.4 |

TABLE 17
45 mg / 6 month Delivered Dose Consistency

| | Liquid - Solid | | |
|---|---|---|---|
| | 100% LA + | Liquid-Liquid | |
| | 50% Polymer | 34.2% LA + 55.0% Polymer | |
| Mixing Cycles | 60 | 20 | 60 |
| N | 6 | 10 | 10 |
| Ave (% target) | 113.0 | 96.3 | 101.2 |
| Stdev (% target) | 1.5 | 3.4 | 2.5 |
| RSD (%) | 1.3 | 3.5 | 2.5 |

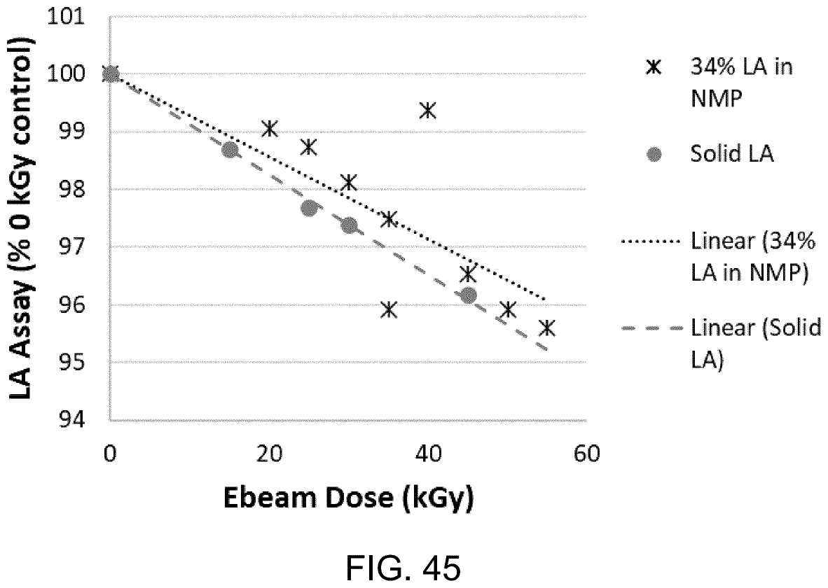

To demonstrate improved stability of liquid-liquid formulations, the following experiments were conducted. Liquid drug syringes were prepared by dissolving LA in NMP and then filled into syringes and packaging in foil pouches. Solid drug syringes were prepared by dissolving LA in water and gently mixing until dissolved. The LA solution was filled into male syringes, lyophilized to remove water, and then packaged in trays. Samples were irradiated by e-beam irradiation at the indicated target dose. The irradiated syringe was delivered and tested for LA content directly (not mixed with a polymer syringe). LA content was then expressed as a % of the non-irradiated control. A linear fit was applied to each data set with the intercept set to 100% (FIG. 45). While there was variability in results for the LA solution, the stability of leuprolide acetate to e-beam irradiation was generally similar or slightly better when in the drug solution than as a solid. This is surprising, as typically chemicals in solution are more reactive (and thus sensitive to degradation) than when in solid form.

### XV. Liquid-Liquid Device/Product Optimization

Variability in certain combination products may impact the delivered constituted product's consistency and critical quality attribute (CQA) results. Two main causes of the variability that prevents consistently passing CQAs are: 1) lack of complete mixing, and 2) device handling procedural deficiencies. Both parameters were addressed for the studied strengths in liquid-liquid formulations when using the syringe embodiment having the syringe connector as described herein.

For lower fill volume strengths (1M/7.5 mg and 3M/22.5 mg), connector flow path occlusions were evaluated in addition to formulation changes. Ultimately, the flow path occlusions did not result in a benefit to the combination product, but formulation changes were effective in increasing the mixing efficiency. For strengths with larger fill volumes (6 m/45 mg and 4M/30 mg) variability in CQAs decreased with device handling optimizations. Overall, the problem of variability in these combination products was solved with a combination of strategies employed to ensure complete mixing and consistent device handling during the mixing and delivery process. These improvements all aimed to increase the robustness and reliability of the products from a CQA testing perspective.

Higher fill volume formulations that did not exhibit mixing efficiency issues at 20 cycles (6M/45 mg and 4M/30 mg) were still showing high variability in the delivered mass that decreased the robustness of the CQAs needed to prove similarity. To address this issue, any source of variability in the handling procedure and device was reduced through Instruction For Use (IFU) changes and verified with several testing runs on the 6M/45 mg strength. For the lower fill volume strengths (3M/22.5 mg), the IFU improvements were not sufficient to limit variability in delivered product CQAs, and formulation composition was changed to increase mixing efficiency. In order to accomplish improved mixing below the prior 60 cycles requirement the syringe B fill volume was increased by moving solvent from the polymer solution to the drug solution, ranging from 17 mg to 46 mg. In some instances, as much solvent as possible was moved from the polymer solution to the drug solution. The increased syringe B fill volume had the effect of increased mixing efficiency, thereby allowing the instant drug-device combination product greater robustness for CQAs.

Two exemplary options were explored for the lower fill volume formulations: 1) device handling procedure improvements to decrease delivered mass variance and 2) reformulating the 3M/22.5 mg strength in order to optimize the mixing for the open (no flow path occlusions) preconnected device system. After initial testing in the male-male device design disclosed herein, it was determined that further optimization was needed for the lower volume strengths (3M/22.5 mg). The lower fill volume formulations of 7.5 mg for 1 month and 22.5 mg for 3 months mixed significantly worse than the higher fill volume formulations (6M/45 mg and 4M/30 mg) in the new two syringe preconnected device systems described herein.

In parallel with the on-going efforts of designing the new custom luer mixing connector (CLMC), (see, e.g., FIG. 35 design with plurality of apertures or openings when in the open position) the device handling procedure was improved in order to reduce variability in the delivered mass as much as possible for the formulations with high polymer solution viscosities (e.g., 6M/45 mg). At higher viscosity, the mixing force required to use these new CLMC device(s), containing any of the proposed mixing elements or occlusion designs inserted within the flow path, was too high for a normal health care provider (HCP) to be expected to mix 20 cycles effectively. This meant that the open fluid path connector (no flow path occlusions) was required for the 6M/45 mg strength. Therefore, the variance had to be controlled in other ways besides CLMC modifications. By making modifications and standardizing the instructions for use (IFU) for the new, instant device, the variance in delivered mass and delivered dose decreased for all strengths.

After the IFU improvements, the flow path occlusions introduced through various CLMC designs that were believed to increase mixing efficiency in earlier stages of development for the 3M/22.5 mg strength were proven to not be effective. For example, the open fluid path performed the same as the connector shown in FIG. 35D which features occlusion structure 360 when compared for content uniformity (USP 905) and IVRT performance. To address the issue of incomplete mixing at 20 cycles for the 3M/22.5 mg formulation, the fill ratio was adjusted to account for the bias in polymer solution holdup within the connector. After making that correction, the variability in delivered product CQAs from these samples that used extremely small fill weight tolerances (+/−1.5 mg) was still slightly too high to reliably pass CQAs and a failure rate calculated from content uniformity data was unacceptable for commercial production. Therefore, the composition of polymer and drug solutions were reformulated as discussed previously herein to increase the volume of syringe B fill by as much as possible without increasing the syringe A viscosity to a level that negatively impacted filling tolerance or manufacturability of the product (the movement of solvent from Syringe A to B increases the polymer to solvent ratio in Syringe A, thus increasing the viscosity in Syringe A). This change increased the mixing efficiency of the formulation while still delivering the same amount of each component in final formulation.

Initial studies showed that the formulation strengths mix with different efficiencies. While mixing flow path occlusions were studied to try and increase mixing efficiency of lower fill volume formulations, some strengths showed slight improvement via visual observations (3M/22.5 mg), while others needed increased mixing force (6M/45 mg) that was considered detrimental to the user experience. Through the iterative testing of various 3D printed CLMCs, the handling and mixing procedure of the drug device combination product was also studied. In those visual mixing observation studies, it was discovered that starting the mixing with the syringe B stroke first (i.e. syringe B into Syringe A) increased the turnover of solution and increased visual mixing in earlier cycle counts in the mixing process. Ultimately, after several rounds of testing (visual and HPLC assay confirmations) there was no appreciable gain in mixing efficiency with any of the flow path occlusions that were evaluated. However, the largest gains in mixing efficiency and product reproducibility were obtained from improved handling and mixing procedures, particularly the reversal in the initial stroke order, wherein the biggest impact was discovered by initial mixing of Syringe B contents (drug solution) into Syringe A contents (polymer solution), as compared to the opposite stroke order used with Solid-Liquid formulations (initially mixing Syringe A into Syringe B).

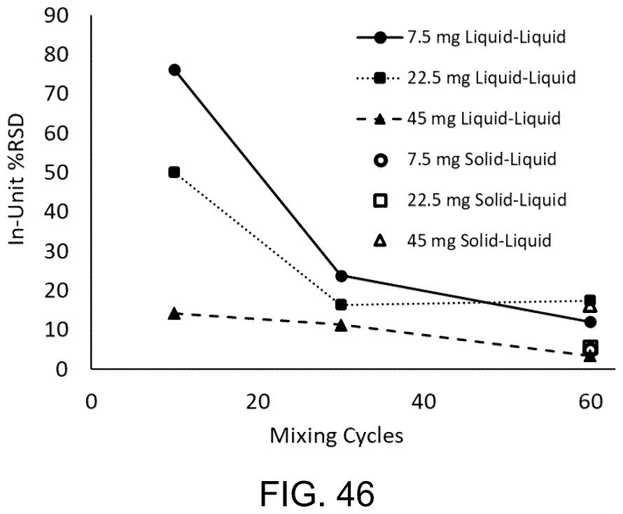

However, for 3M/22.5 mg, the improvements in the aforementioned handling instructions were insufficient to fully resolve the CQA and reproducibility concerns. This led to additional changes, where the formulation components were altered in order to increase mixing while still delivering the same final mixed product after the correct number of mixing cycles. As discussed previously, this was accomplished by moving as much solvent as possible from Syringe A to Syringe B without increasing the Syringe A solution viscosity to an unacceptable level for filling operations. Notably, this same strategy cannot be equivalently applied to the higher dosage strength (6M/45 mg) because there is not enough solvent in the product overall to move any more out of Syringe A. FIG. 46 shows relative mixing efficiency for various formulations including both liquid-liquid formulations and solid-liquid formulations in an exemplary device connector. Results are also included in Table 18 below.

TABLE 18
Liquid-Liquid Mixing Efficiency (In-Unit % RDS)

| | Cycles | | |
|---|---|---|---|
| | 10 | 30 | 60 |
| 7.5 mg | 76% | 24% | 12% |
| 22.5 mg | 50% | 16% | 17% |
| 45 mg | 14% | 11% | 3% |

With evidence that the strengths did not mix equivalently, the mixing procedure was further altered in attempts to increase mixing efficiency. At the same time, several factors that may have negatively impacted mixing efficiency were likewise evaluated to ascertain their impact on the mixing dynamics in the device system and limit the negative impacts they may have had on the variability of final delivered product CQAs (delivered mass, CP assay, Delivered dose (mg LA) and IVRT). To estimate mixing variability, Content Uniformity (CU) was tested with a passing acceptance value indicating a more mixed product. Additionally, In-Vitro Release testing (IVRT) variability of replicates was tested to better understand product homogeneity. These methods were employed to estimate the success or failure of these changes. At this point using the CLMCs and before any optimization to the original device as illustrated in FIG. 13, the instructions for use were as follows:
1) Install syringe flange extenders on both syringes.
2) With the syringe luer tip pointed up, remove Syringe A tip cap and attach syringe A to connector. Discard tip cap.
3) With the syringe luer tip pointed up, remove Syringe B tip cap and attach syringe B to connector's beveled side. Discard tip cap.
4) Holding syringes in a horizontal position, transfer liquid contents of syringe B into Syringe A. Thoroughly mix the product by pushing contents back and forth between both syringes to obtain a uniform suspension.
   a. One complete mixing cycle is defined as one complete push of the plunger for syringe B and one complete push of the plunger for syringe A.
   b. Mix to the predefined number of cycles at approximately 1 cycle per second.
5) After mixing, hold the mixing assembly vertically with syringe B on the bottom. The syringes should remain securely coupled. Push all the mixed product into Syringe B by depressing the syringe A plunger with your thumb. Secure the connector with your 4th and 5th fingers prior to disconnecting.
6) While ensuring Syringe A plunger is fully pushed down, hold the connector, and unscrew it from syringe B. Syringe A will remain attached to the connector.
   a. Do not pull negative pressure on syringe B plunger while disconnecting.
   b. Do not purge air bubbles at this step. Wait until needle is attached to purge any air incorporated into the product during mixing.
7) Attach needle. Remove needle cap.
8) Record the mass of syringe B with the mixed product, needle, and flange extender. Express mixed product into a receiving vessel. Record the mass of syringe B after injection to calculate delivered mass by difference.

New instructions after further improvements included the following:
1) Install syringe flange extenders on both syringes.
2) Activate connector.
3) Holding syringes in a horizontal position, transfer liquid contents of syringe B into Syringe A. Thoroughly mix the product by pushing contents back and forth between both syringes to obtain a uniform suspension.
   a. One complete mixing cycle is defined as one complete push of the plunger for syringe B and one complete push of the plunger for syringe A.
   b. Mix 20 cycles at approximately 1 cycle per second.
4) After mixing, hold the device vertically with syringe B on the bottom. The syringes should remain securely coupled. Push all the mixed product into Syringe B by depressing Syringe A plunger with your thumb. Secure the connector with your 4th and 5th fingers prior to disconnecting.
5) While ensuring Syringe A plunger is fully pushed down, hold the connector, and unscrew it from syringe B. Syringe A will remain attached to the connector.
   a. Pull negative pressure on syringe B plunger while disconnecting.
   b. Do not purge air bubbles from Syringe B.
6) Securely attach the needle so it is fully engaged in the luer lock. Remove needle cap.
7) Express mixed product into the receiving vessel as needed for testing purposes.
8) Activate needle safety cover and dispose of all waste per procedures.

Using this procedure, results are shown in Tables 19-20 below.

TABLE 19
Results of IFU robustness testing on a single lab scale lot of the
45 mg formulation with altered steps in sample preparation process
% of target Delivered Dose Content uniformity (% of 45 mg LA delivered)

| | Proto- | Proto- | | Prototype | Prototype B |
|---|---|---|---|---|---|
| | type B | type A | CLMC | Air Gap 20 | into A 60 |
| Replicate | into A | into B | B into A | Cycles | Cycles |
| N | 10 | 10 | 10 | 10 | 10 |
| AVG | 96.3 | 102.3 | 95.1* | 98.2 | 101.2 |
| STDEV | 3.4 | 4.7 | 2.7 | 3.3 | 2.5 |
| CU | 10.4 | 12.2 | 10.0* | 8.1 | 5.9 |
| (AV < 15 | | | | | |

TABLE 20
Additional Results
% of target CP Assay Content uniformity (% of 12% w/w LA)

| | Proto- | Proto- | | Prototype | Prototype B |
|---|---|---|---|---|---|
| | type B | type A | CLMC | Air Gap 20 | into A 60 |
| Replicate | into A | into B | B into A | Cycles | Cycles |
| N | 10 | 10 | 10 | 10 | 10 |
| AVG | 94.0 | 98.6 | 99.2* | 97.1 | 99.5 |
| STDEV | 3.3 | 4.6 | 3.0 | 4.1 | 1.4 |
| CU | 12.5 | 11.1 | 7.3* | 11.2 | 3.3 |
| (AV < 15 | | | | | |

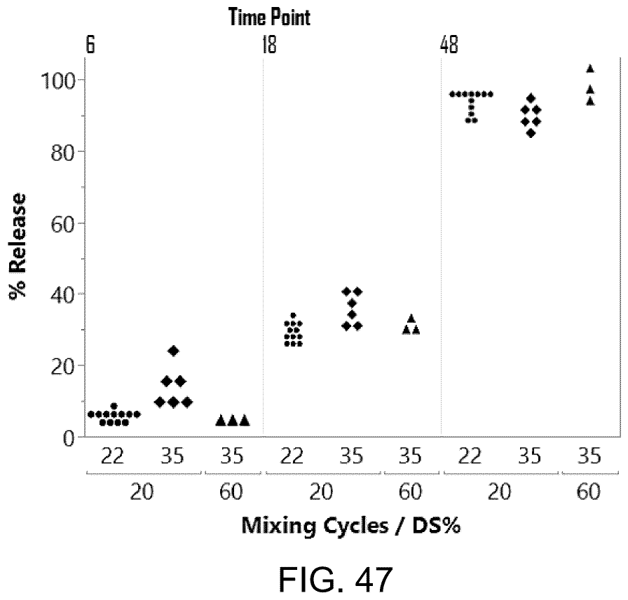

FIG. 47 shows In-Vitro release performance of the 35% w/w leuprolide acetate drug solution and the lower concentration 24% w/w leuprolide acetate drug solution formulation for the 22.5 mg (3-month liquid-liquid) formulation. Both liquid-liquid formulations were tested for mixing efficiency. The 35% liquid-liquid formulation was mixed at 60 cycles and the reduced 20 mixing cycles for comparison. The 24% liquid-liquid formulation was mixed at only 20 cycles. Mixing efficiency was defined by LA release at 6, 18, and 38 hrs after mixing each formulation at 20 or 60 cycles. A tight distribution of replicates at the initial 6 hr release point has been identified as being indicative of a well-mixed product. This observation is supported by the high degree of reproducibility in the first time point for the release of the 35% formulation with 60 mixing cycles. In more detail, the relative standard deviation for the 35% formulation at 20 mixing cycles is more than 5 times the same formulation with 60 mixing cycles. For comparison the mixing efficiency appears high enough for the 24% formulation to show comparable reproducibility to the 35% formulation at 60 mixing cycles, indicating a significant improvement in the mixedness of the final delivered product. Results are summarized in Table 21 below.

TABLE 21
Results Summary
Results Summary Table

| Formulation | Time Point | Average | % RSD |
|---|---|---|---|
| T1 | 6 h | 4.3 | 9.2 |
| new formulation | 18 h | 26.9 | 3.5 |
| 20 Mixing Cycles | 48 h | 92.7 | 3.4 |
| T2 | 6 h | 13.9 | 41.4 |
| old formulation | 18 h | 35.3 | 12.4 |
| 20 Mixing Cycles | 48 h | 89.3 | 4.0 |
| T3 | 6 h | 4.7 | 7.0 |
| old formulation | 18 h | 30.8 | 4.6 |
| 60 Mixing Cycles | 48 h | 97.8 | 4.9 |

The initial formulation using 35% w/w drug substance in NMP did not mix consistently enough at 20 cycles to provide a fill mass range that was manufacturable.

TABLE 22
34% drug solution syringe B formulation table

| | | Proposed 34% LA | | | |
|---|---|---|---|---|---|
| | | Syringe B | | 24% LA Syringe B | |
| | | | mg | | mg |
| Component | Excipient | % w/w | Delivered | % w/w | Delivered |
| (A) Delivery | 75:25 PLG | 42.3 | 158.6 | 42.3 | 158.6 |
| System | NMP | 40.3 | 151.1 | 32.7 | 122.6 |
| (B) Drug | LA | 6.0 | 22.5 | 6.0 | 22.5 |
| Substance | NMP | 11.4 | 42.8 | 19.0 | 71.3 |

| | | | |
|---|---|---|---|
| Total NMP | 51.7 | 193.9 | 51.7 |

| | |
|---|---|
| Final Delivered Product | 375 mg |

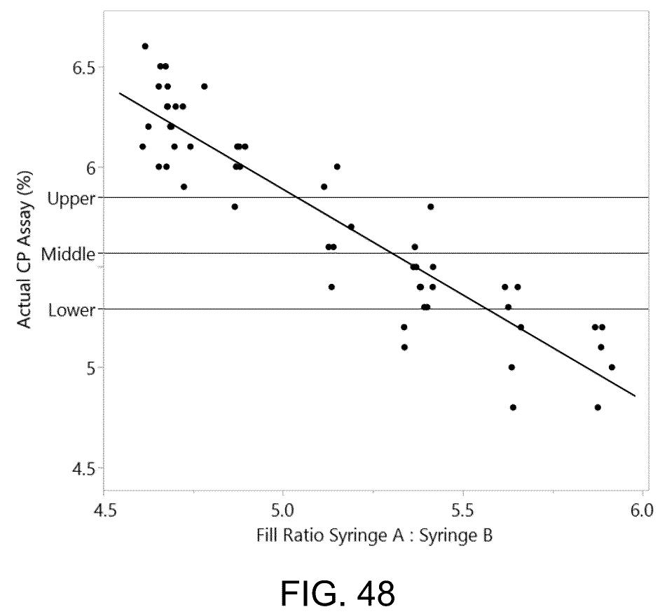

The formulations used to generate FIG. 48 used the same syringe A and syringe B solutions for filling and the fill targets were extremely close together (about 4 mg difference between target fills with a +/−1.5 mg range allowed on each side of each fill target). CP assay in the mixed product was plotted as a function of syringe A to syringe B fill weight ratio. The variance observed from the individual unit constituted product assay (% w/w leuprolide) results shows that the specification range is too tight to be able to reliably fill the 35% w/w formulation at commercial scale and keep it within +/−5% of the target CP assay, shown as Upper and Lower limits on the Y axis of FIG. 48. In order to produce a robust and manufacturable product with wider fill tolerances, it was decided to reformulate the syringe A and B component concentrations by moving solvent from A into B (24% w/w drug solution in Syringe B).

The increase in the volume of syringe B makes the fill tolerance less sensitive; for example +/−5 mg is a lower % of the total for a 150 mg target than the same fill tolerance about a 100 mg target. The reasoning behind these decisions was based on the available data showing the higher dose formulations were demonstrably better mixed after comparable numbers of mixing cycles. A review of these formulations showed that they had larger drug solution fill volume relative to the polymer solution fill volume which appeared to correlate with improved mixing. A controlling factor in this hypothesis is the device system and the holdup volume, or dead space, in the connector and non-dosing syringe. This holdup volume is larger than the fill volume of the small volume formulations including 3M/22.5 mg. Without sufficient volume to occupy this void volume, component mixing is increasingly difficult and inefficient. Additionally, mixing small volumes of liquids is made more difficult when those liquids are non-Newtonian with relatively high viscosities. Diluting the drug solution by redistributing NMP from the polymer solution to the drug solution thereby increases the volume of the drug solution to thereby improve the fill accuracy, as well as lowering the viscosity of the drug solution to improve mixing while minimally impacting the polymer solution viscosity.

Using the new formulation and relevant fill weight tolerances, the standard deviations of the final mixed product CQAs are acceptable and show that the reproducibility of the product at 20 mixing cycles is greatly improved after reformulating. Additionally, a challenge with the 35% formulation was that the polymer solution was preferentially held-up in the device which caused an over-delivery of drug proportionally. The new formulation composition at 24% LA drug solution does not show this same phenomenon, suggesting that the new formulation is closer to complete mixing at 20 cycles.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

1-81. (canceled)

82. A syringe coupler configured to couple to a first syringe and a second syringe, the syringe coupler comprising:
a valve assembly having a first portion and a second portion that are displaceable relative to one another between at least a first position and a second position, wherein the first portion and the second portion comprise respective internal flow ports and syringe receiving portions, wherein the internal flow port of the first portion is configured to couple to the first syringe and the internal flow port of the second portion is configured to couple to the second syringe, wherein the second portion comprises a sealing element that is displaceable with and in fixed relative position to the second syringe, wherein the sealing element comprises an upstanding portion that defines a first sealed area that is aligned with the internal flow port of the second portion,
wherein in the first position of the valve assembly:
the upstanding portion of the sealing element provides a contact seal against the first portion; and
the internal flow port of the second portion and the first sealed area are offset from the internal flow port of the first portion, and
wherein in the second position of the valve assembly:
the upstanding portion of the sealing element provides a contact seal against the first portion; and
the internal flow port of the second portion and the first sealed area are aligned or substantially aligned with and in fluid communication with the internal flow port of the first portion.

83. The syringe coupler of claim 82, wherein the upstanding portion defines a second sealed area that is separated from the first sealed area by a septum,
wherein in the first position of the valve assembly, the second sealed area is aligned with the internal flow port of the first portion, and
wherein in the second position of the valve assembly, the second sealed area is offset from the internal flow port of the first portion.

84. The syringe coupler of claim 82, wherein in the first position of the valve assembly, the upstanding portion is configured to prevent one or both of:
travel of fluid between the internal flow ports of the first and second portions; or
leakage of fluid from either or both of the first and second syringes through the syringe coupler.

85. The syringe coupler of claim 82, wherein the valve assembly comprises a user interface operable to receive a force from a user and transmit the force to the sealing element.

86. The syringe coupler of claim 82, wherein the first portion of the valve assembly comprises a rotatable member that is free to rotate when the valve assembly is in the second position, wherein the selectively rotatable member is configured to engage the first syringe.

87. A syringe-to-syringe mixing system comprising:
a first syringe;
a second syringe;
a valve assembly having a first portion and a second portion that are displaceable relative to one another between at least a first position and a second position, wherein the first portion and the second portion comprise respective internal flow ports and syringe receiving portions, wherein the internal flow port of the first portion is coupled to the first syringe and the internal flow port of the second portion is coupled to the second syringe, wherein the second portion comprises a sealing element that is displaceable with and in fixed relative position to the second syringe, wherein the sealing element comprises an upstanding portion that defines a first sealed area that is aligned with the internal flow port of the second portion,
wherein in the first position of the valve assembly:
the upstanding portion of the sealing element provides a contact seal against the first portion; and
the internal flow port of the second portion and the first sealed area are offset from the internal flow port of the first portion, and
wherein in the second position of the valve assembly:
the upstanding portion of the sealing element provides a contact seal against the first portion; and
the internal flow port of the second portion and the first sealed area are aligned or substantially aligned with and in fluid communication with the internal flow port of the first portion to permit mixing of contents of the first and second syringes.

88. The syringe-to-syringe mixing system of claim 87, wherein at least one of the first syringe or the second syringe contains a liquid component.

89. The syringe-to-syringe mixing system of claim 87, wherein one of the first syringe or the second syringe contains a liquid component, and wherein the other of the first syringe or the second syringe contains a lyophilized component.

90. The syringe-to-syringe mixing system of claim 87, wherein the first syringe and the second syringe each contains a liquid component.

91. The syringe-to-syringe mixing system of claim 87, wherein at least one of the first syringe or the second syringe contains a Gonadotropin Releasing Hormone (GnRH) agonist or antagonist.

92. The syringe-to-syringe mixing system of claim 91, wherein the GnRH agonist comprises leuprolide or a pharmaceutically acceptable salt thereof.

93. The syringe-to-syringe mixing system of claim 92, wherein the GnRH agonist comprises leuprolide acetate.

94. The syringe-to-syringe mixing system of claim 91, wherein the GnRH agonist is provided in a lyophilized or liquid component.

95. The syringe-to-syringe mixing system of claim 94, wherein the GnRH agonist comprises leuprolide or a pharmaceutically acceptable salt thereof.

96. The syringe-to-syringe mixing system of claim 95, wherein the GnRH agonist comprises leuprolide acetate.

97. The syringe-to-syringe mixing system of claim 87, wherein the first syringe and the second syringe each contain a GnRH agonist or antagonist.

98. The syringe-to-syringe mixing system of claim 97, wherein the GnRH agonist or antagonist contained within the first syringe is provided in a liquid component.

99. The syringe-to-syringe mixing system of claim 98, wherein the GnRH agonist or antagonist contained within the first syringe is provided in a first liquid component, and wherein the GnRH agonist or antagonist contained within the second syringe is provided in a second liquid component.

100. The syringe-to-syringe mixing system of claim 97, wherein the first syringe and the second syringe each contain leuprolide or a pharmaceutically acceptable salt thereof.

101. The syringe-to-syringe mixing system of claim 87, wherein the upstanding portion of the valve assembly defines a second sealed area that is separated from the first sealed area by a septum,
wherein in the first position of the valve assembly, the second sealed area is aligned with the internal flow port of the first portion, and
wherein in the second position of the valve assembly, the second sealed area is offset from the internal flow port of the first portion.

102. The syringe-to-syringe mixing system of claim 87, wherein in the first position of the valve assembly, the contact seal formed between the upstanding portion and the first portion is configured to prevent one or both of:
travel of fluid between the internal flow ports of the first and second portions of the valve assembly; or
leakage of fluid from either or both of the first and second syringes through the syringe coupler.

103. The syringe-to-syringe mixing system of claim 87, wherein the valve assembly comprises a user interface operable to receive a force from a user and transmit the force to the sealing element of the valve assembly.

104. The syringe-to-syringe mixing system of claim 87, wherein the first portion of the valve assembly comprises a rotatable member that is free to rotate when the valve assembly is in the second position, wherein the selectively rotatable member engages the first syringe.
